(19) United States
(12) Patent Application Publication
(10) Pub. No.: US 2025/0092404 A1
(43) Pub. Date: Mar. 20, 2025

(54) PIKFYVE ANTISENSE OLIGONUCLEOTIDES

(71) Applicant: AcuraStem Incorporated, Pasadena, CA (US)

(72) Inventors: Wen-Hsuan CHANG, Pasadena, CA (US); Emily Elizabeth LEE, Pasadena, CA (US)

(21) Appl. No.: 18/818,329

(22) Filed: Aug. 28, 2024

Related U.S. Application Data
(63) Continuation of application No. 17/808,247, filed on Jun. 22, 2022, now Pat. No. 12,104,159.
(60) Provisional application No. 63/202,717, filed on Jun. 22, 2021.

(51) Int. Cl. C12N 15/113; A61P 25/00

(52) U.S. Cl. CPC C12N 15/1137; A61P 25/00; C12N 2310/11; C12N 2310/31; C12N 2310/315; C12N 2310/321; C12N 2310/3341; C12N 2310/341; C12N 2310/345; C12N 2310/346; C12N 2320/32

(57) ABSTRACT

The present invention relates to PIKFYVE antisense oligonucleotides (ASOs), pharmaceutical compositions containing them, and methods for treating, inhibiting, suppressing, and preventing neurological diseases with them.

This application claims the benefit of U.S. Patent Application No. 63/202,717, filed Jun. 22, 2021, which is hereby incorporated by reference.

### FIELD OF THE INVENTION

The present invention relates to PIKFYVE antisense oligonucleotides (ASOs), pharmaceutical compositions containing them, and methods for treating, inhibiting, suppressing, and preventing neurological or neurodegenerative diseases with them.

### BACKGROUND OF THE INVENTION

Many neurodegenerative disorders in patients are difficult to effectively treat, especially where the pathology of a neurodegenerative disorder in a particular patient is not completely understood.

International Publication No. WO 2016/210372 discloses a method of treating a neurodegenerative disease by administering a PIKFYVE inhibitor.

There remains a need for effective treatments for many neurodegenerative disorders, such as amyotrophic lateral sclerosis (ALS) and frontotemporal dementia (FTD).

### SUMMARY OF THE INVENTION

The present invention relates to PIKFYVE antisense oligonucleotides (ASOs), pharmaceutical compositions containing them, and their use in the treatment of neurodegenerative disorders.

One embodiment is a single stranded ASO that suppresses the expression of PIKFYVE, wherein the ASO has a nucleobase sequence that comprises at least 12 or 15 consecutive nucleobases of any of the nucleobase sequences of SEQ ID NO: 1-500. The nucleobase sequence of the ASO can comprise up to 30, 25, 24, 23, 22, 21, or 20 consecutive nucleobases of any of the nucleobase sequences of SEQ ID NO: 1-500. The ASO can also be any of SEQ ID NO: 1-500.

Another embodiment is an oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or at least 20 consecutive nucleobases of any of the nucleobase sequences of SEQ ID NO: 1-500. The oligonucleotide can comprise up to 25, 24, 23, 22, 21, or 20 consecutive nucleobases of any of the nucleobase sequences of SEQ ID NO: 1-500.

In certain embodiments, at least one internucleoside linkage is a modified internucleoside linkage, and the modified internucleoside linkage may be a phosphorothioate internucleoside linkage or a phosphodiester internucleoside linkage. At least one of the nucleosides may also be a modified nucleobase.

In other embodiments, at least one nucleoside of the ASO may be a modified sugar moiety, where that modified sugar moiety can be a bicyclic sugar moiety, or the modified sugar moiety may comprise a 2′-O-methoxyethyl group. In certain aspects, the bicyclic sugar moiety comprises a 4′-CH(R)—O-2′ bridge where the R group is, independently, H, C₁₋₁₂ alkyl, or a protecting group.

In yet other embodiments, the ASO is a gapmer (e.g., a MOE gapmer), where a gap segment may consist of 8 to 12 linked deoxynucleosides, a 5′ wing segment consisting of 3 to 5 linked nucleosides, and a 3′ wing segment consisting of 3 to 5 linked nucleosides. In certain aspects, the gap segment may be positioned between the 5′ wing segment and the 3′ wing segment, where a nucleoside of each wing segment comprises a modified sugar moiety (e.g., one with a 2′-O-methoxyethyl group).

In other embodiments, the oligonucleotide consists of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or at least 20 consecutive nucleobases of any of the nucleobase sequences of SEQ ID NO: 1-500.

Another embodiment is a pharmaceutical composition comprising a PIKFYVE ASO of the present invention and one or more pharmaceutically acceptable carriers, diluents, and/or excipients. In one embodiment, the pharmaceutical composition is suitable for parenteral administration, such as intracerebroventricular injection or intrathecal administration.

Yet another embodiment is a method of inhibiting, suppressing, or preventing expression of PIKFYVE in a patient (such as one having a neurological or neurodegenerative disease) by administering (for example by, intracerebroventricular injection or intrathecal administration) a PIKFYVE ASO or a pharmaceutical composition described herein (for instance, an effective amount thereof) to the patient.

Yet another embodiment is a method of treating a subject having a neurological or neurodegenerative disease by administering a therapeutically effective amount of a PIKFYVE ASO or a pharmaceutical composition described herein. In one embodiment, the disease is amyotrophic lateral sclerosis (ALS) (e.g., C9orf72-associated ALS). In another embodiment, the disease is frontotemporal dementia (FTD), such as FTD with TDP-43 pathology or FTD with tau pathology. In yet another embodiment, the disease is C9orf72-associated FTD (C9-FTD). In yet another embodiment, the disease is microtubule-associated protein tau (MAPT)-associated FTD (MAPT-FTD), such as FTD with the V337M MAPT mutation.

Yet another embodiment is a method of treating a subject having a PIKFYVE disease or disorder by administering a therapeutically effective amount of a PIKFYVE ASO or a pharmaceutical composition described herein.

### BRIEF DESCRIPTION OF THE FIGURES

A more complete appreciation of the invention and many of the attendant advantages thereof will be readily obtained as the same becomes better understood by reference to the following detailed description when considered in connection with the accompanying figures, wherein:

FIG. 1 shows a PIKFYVE ASO screen in Hela cells, measuring the relative mRNA expression levels of ASO 1-33 (SEQ ID NO: 1-33) against a control (NCASO).

FIG. 2 is a bar graph showing the suppressive effect of various PIKFYVE ASOs in neonate transgenic hPIKFYVE BAC mice.

FIG. 3 are bar graphs showing the change in PIKFYVE mRNA and PIKFYVE protein in non-human primates (NHP) with and without treatment of ASO-520 or artificial cerebrospinal fluid (aCSF).

FIG. 4A is a graph showing the percent survival of control motor neurons in the presence of a non-coding ASO (NC ASO) or C9ALS patient-derived motor neurons in the presence of (i) a NC ASO or (ii) AS-20 (SEQ ID NO: 20).

FIG. 4B is a bar graph showing the hazard ratio for control motor neurons in the presence of a NC ASO or C9ALS patient-derived motor neurons in the presence of (i) a NC ASO or (ii) AS-520 (SEQ ID NO: 520).

FIG. 5A is a graph showing the probability of survival for FTD patient-derived cortical neurons with MAPT V337V or V337M in the present of NC ASO or AS-520 (SEQ ID NO: 520).

FIG. 5B is a bar graph showing the hazard ratio for cortical neurons from control, C9orf72-associated FTD (C9-FTD), sporadic FTD (sFTD), and microtubule-associated protein tau (MAPT)-associated FTD (MAPT-FTD) patients treated with NC ASO or AS-520.

### DETAILED DESCRIPTION OF THE PREFERRED EMBODIMENTS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting. The terminology used herein is for the purpose of describing particular embodiments only and is not intended to be limiting.

### Definitions

The terms “comprise(s),” “include(s),” “having,” “has,” “can,” “contain(s),” “may” and variants thereof, as used herein, are intended to be open-ended transitional phrases, terms, or words that do not preclude the possibility of additional acts or structures.

The singular forms “a,” “an” and “the” include plural references unless the context clearly dictates otherwise.

The present disclosure also contemplates other embodiments “comprising,” “consisting of” and “consisting essentially of,” the embodiments or elements presented herein, whether explicitly set forth or not.

As used herein, “2′-deoxynucleoside” means a nucleoside comprising a 2′-H(H) furanosyl sugar moiety, as found in naturally occurring deoxyribonucleic acids (DNA) and a nucleobase. In certain embodiments, a 2′-deoxynucleoside may comprise a modified nucleobase and a furanosyl sugar moiety or may comprise an RNA nucleobase (uracil) and a furanosyl sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “antisense molecule” means an oligomeric nucleic acid or oligomeric duplex capable of achieving at least one antisense activity.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety. As used herein, “bicyclic nucleoside” or “BNA” means a nucleoside comprising a bicyclic sugar moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T); adenine (A) and uracil (U); cytosine (C) and guanine (G); and 5-methylcytosine (mC) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” Unless otherwise indicated, “gapmer” refers to a sugar motif. Unless otherwise indicated, the sugar moieties of the nucleosides of the gap of a gapmer are unmodified 2′-deoxyfuranosyl. Thus, the term “MOE gapmer” indicates a gapmer having a sugar motif of 2′-MOE nucleosides in both wings and a gap of 2′-deoxynucleosides. Unless otherwise indicated, a MOE gapmer may comprise one or more modified internucleoside linkages and/or modified nucleobases and such modifications do not necessarily follow the gapmer pattern of the sugar modifications. Table 2, below, provides exemplary MOE-gapmers.

In certain embodiments, oligonucleotides comprise one or more type of modified sugar and/or unmodified sugar moiety arranged along the oligonucleotide or region thereof in a defined pattern or sugar motif. In certain instances, such sugar motifs include, but are not limited to, any of the sugar modifications discussed herein.

In certain embodiments, modified oligonucleotides comprise or consist of a region having a gapmer motif, which is defined by two external regions or “wings” and a central or internal region or “gap.” The three regions of a gapmer motif include the “5′ wing”, the “gap” and the “3′ wing” which form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, each nucleoside of each wing of a gapmer is a modified nucleoside.

In certain embodiments, the gap of a gapmer comprises 7-12 nucleosides (e.g., 10 nucleosides). In certain embodiments, each nucleoside of the gap of a gapmer is an unmodified 2′-deoxy nucleoside.

In certain embodiments, the gapmer is a deoxy gapmer. In embodiments, the nucleosides on the gap side of each wing/gap junction are unmodified 2′-deoxy nucleosides and the nucleosides on the wing sides of each wing/gap junction are modified nucleosides. In certain embodiments, each nucleoside of the gap is an unmodified 2′-deoxy nucleoside. In certain embodiments, each nucleoside of each wing of a gapmer is a modified nucleoside.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified oligonucleotide comprises the same 2′-modification.

“Inhibit” as used herein refers to the ability to substantially antagonize, prohibit, prevent, suppress, restrain, slow, disrupt, alter, eliminate, stop, or reverse the progression or severity of the activity of a particular agent (e.g., infectious agent) or disease.

As used herein, the term “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate linkage” is a modified internucleoside linkage in which one of the non-bridging oxygen atoms of a phosphodiester internucleoside linkage is replaced with a sulfur atom.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include, but are not limited to, phosphates, which contain a phosphodiester bond (also referred to as unmodified or naturally occurring linkages), phosphotriesters, methylphosphonates or other alkylphosphonates, phosphoramidates, and phosphorothioates, and phosphorodithioates. Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Representative internucleoside linkages having a chiral center include, but are not limited to, alkylphosphonates and phosphorothioates. Modified oligonucleotides comprising internucleoside linkages having a chiral center can be prepared as populations of modified oligonucleotides comprising stereo-random internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereo-random. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003); Wan et al., Nuc. Acid. Res. 42, 13456 (2014); Chapter 10 of Locked Nucleic Acid Aptamers in Nucleic Acid and Peptide Aptamers: Methods and Protocols v 535, 2009 by Barciszewski et al., editor Gunter Mayerand; and WO 2017/015555. In certain embodiments, a population of modified oligonucleotides is enriched for modified oligonucleotides having at least one indicated phosphorothioate in the (Sp) configuration. In another embodiment, a population of modified oligonucleotides is enriched for modified oligonucleotides having at least one indicated phosphorothioate in the (Rp) configuration.

As used herein, “MOE” means methoxyethyl. “2′-MOE” means a —OCH₂CH₂OCH₃ group at the 2′ position of a furanosyl ring.

A “neurological disease” is any disease that causes electrical, biochemical, or structural abnormalities in the brain, spine, or neurons. For example, a neurological disease may be a neurodegenerative disease. The neurodegenerative disease may result in motor neuron degeneration, for example. The neurological disease may be amyotrophic lateral sclerosis (ALS), Huntington's disease, Alzheimer's disease, or frontotemporal dementia, for example. Further examples of neurological diseases include, but are not limited to, Parkinson's disease, multiple sclerosis, peripheral myopathy, Rasmussen's encephalitis, attention deficit hyperactivity disorder, autism, central pain syndromes, anxiety, and/or depression, for example.

The neurological disease may be associated with aberrant endosomal trafficking. For example, endosomal pathways and endosomes are necessary components for the recycling or breakdown of membrane-bound proteins, trafficking of Golgi-associated proteins, and the extracellular release of proteins in exosomes. These processes aid neurotransmission and drive a balance between recycling and degradation of synaptic vesicles or neurotransmitter receptors, for example.

The neurological disease may be associated with aberrant lysosome degradation. Alterations in the lysosome degradation may be present in the neurological disease, such as a neurodegenerative disease. Cathepsin imbalance during aging and age-related diseases may provoke deleterious effects on central nervous system (CNS) neurons and lysosomes may be sites for the unfolding and partial degradation of membrane proteins or their precursors that subsequently become expelled from a cell, or are released from dead cells and accumulate as pathological entities.

A health care professional may diagnose a subject as having a disease associated with motor neuron degeneration by the assessment of one or more symptoms of motor neuron degeneration. To diagnose a neurological disease, a physical exam may be followed by a thorough neurological exam. The neurological exam may assess motor and sensory skills, nerve function, hearing and speech, vision, coordination and balance, mental status, and changes in mood or behavior. Non-limiting symptoms of a disease associated with a neurological disease may be weakness in the arms, legs, feet, or ankles; slurring of speech; difficulty lifting the front part of the foot and toes; hand weakness or clumsiness; muscle paralysis; rigid muscles; involuntary jerking or writing movements (chorea); involuntary, sustained contracture of muscles (dystonia); bradykinesia; loss of automatic movements; impaired posture and balance; lack of flexibility; tingling parts in the body; electric shock sensations that occur with movement of the head; twitching in arm, shoulders, and tongue; difficulty swallowing; difficulty breathing; difficulty chewing; partial or complete loss of vision; double vision; slow or abnormal eye movements; tremor; unsteady gait; fatigue; loss of memory; dizziness; difficulty thinking or concentrating; difficulty reading or writing; misinterpretation of spatial relationships; disorientation; depression; anxiety; difficulty making decisions and judgments; loss of impulse control; difficulty in planning and performing familiar tasks; aggressiveness; irritability; social withdrawal; mood swings; dementia; change in sleeping habits; wandering; and change in appetite.

Tests may be performed to rule diseases and disorders that may have symptoms similar to those of neurological diseases, measure muscle involvement, assess neuron degeneration. Non-limiting examples of tests are electromyography (EMG); nerve conduction velocity study; laboratory tests of blood, urine, or other substances; magnetic resonance imaging (MRI); magnetic resonance spectroscopy; muscle or nerve biopsy; transcranial magnetic stimulation; genetic screening; x-rays; fluoroscopy; angiography; computed tomography (CT); positron emission tomography; cerebrospinal fluid analysis; intrathecal contrast-enhanced CT scan; electroencephalography; electronystagmography; evoked response; polysomnogram; thermography; and ultrasound. A health care professional may also assess the patient's family history of diseases associated with motor neuron degeneration and make a diagnosis in part based on a familial history of neurological diseases. A healthcare professional may diagnose a disease associated with neurological disease in a subject after the presentation of one or more symptoms.

Neurodegenerative diseases result in the progressive destruction of neurons that affects neuronal signaling. For example, a neurodegeneration may be amyotrophic lateral sclerosis, Alzheimer's disease, Huntington's disease, Friedreich's ataxia, Lewy body disease, Parkinson's disease, spinal muscle atrophy, primary lateral sclerosis, progressive muscle atrophy, progressive bulbar palsy, and pseudobulbar palsy.

Diseases associated with motor neuron degeneration may be a condition that results in the progressive destruction of motor neurons that interferes with neuronal signaling to the muscles, leading to muscle weakness and wasting. In healthy individuals, upper motor neurons transmit signals from the brain to lower motor neurons in the brain stem and spinal cord, which then transmit the signal to the muscles to result in voluntary muscle activity. The destruction of upper and lower motor neurons affects activity such as breathing, talking, swallowing, and walking, and overtime these functions can be lost. Examples of motor neuron diseases include, but are not limited to, amyotrophic lateral sclerosis, primary lateral sclerosis, progressive muscle atrophy, progressive bulbar palsy, and pseudobulbar palsy.

Neuronal hyperexcitability may occur when receptors for the excitatory neurotransmitter glutamate (glutamate receptors) such as the NMDA receptor and AMPA receptor are over-activated by excess glutamate or by other compounds or neurotransmitters acting on the glutamate receptors. Excitotoxicity may result from neuronal hyperexcitability. Excitotoxicity is the pathological process by which nerve cells are damaged or killed by excessive stimulation. The excessive stimulation allows high levels of calcium ions (Ca²⁺) to enter the cell. Ca²⁺ influx into cells activates a number of enzymes, including phospholipases, endonucleases, and proteases such as calpain. These enzymes can damage cell structures such as components of the cytoskeleton, membrane, and DNA.

Neuronal hyperexcitability may be involved in spinal cord injury, stroke, traumatic brain injury, hearing loss (through noise overexposure or ototoxicity), epilepsy, painful neuropathies, attention deficit hyperactivity disorder, autism, central pain syndromes, neurodegenerative diseases, multiple sclerosis, Alzheimer's disease, amyotrophic lateral sclerosis (ALS), Parkinson's disease, frontotemporal dementia, schizophrenia, Rasmussen's encephalitis, Huntington's disease, alcoholism or alcohol withdrawal and especially over-rapid benzodiazepine withdrawal, and also Huntington's disease. Other common conditions that cause excessive glutamate concentrations around neurons are hypoglycemia. Blood sugars are the primary glutamate removal method from inter-synaptic spaces at the NMDA and AMPA receptor site.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. As used herein, an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase or modified nucleobase. A “5-methylcytosine” or “mC” is a modified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethylcytosine, xanthine, hypoxanthine, 2-aminoadenine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a continuous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. The internucleoside linkages may be any described herein. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

“PIKFYVE”, also known in the art as “phosphatidylinositol-3-phosphate 5-kinase type III” or “PIPKIII”, is a FYVE finger-containing phosphoinositide kinase encoded by the PIKFYVE gene. PIKFYVE is a highly evolutionarily conserved lipid kinase and also has protein kinase activity, which regulates endomembrane homeostasis and plays a role in the biogenesis of endosome carrier vesicles from early endosomes. PIKFYVE-mediated conversion of PI3P to PI(3,5)P₂ blocks recruitment of the protein EEA1. The recruitment is blocked, because PIP3 is needed to form a platform with RAB5 that enables anchoring of EEA1 to early endosomes. EEA1 then drives fusion with endocytic and other endosomal vesicles.

As used herein a “PIKFYVE disease or disorder” includes lysosomal degradation diseases and disorders mediated by PIKFYVE. For example, the a PIKFYVE disease or disorder includes, but is not limited to, amyloid diseases (such as Alzheimer's disease, Parkinson's disease, Huntington's disease, type 2 diabetes, diabetic amyloidosis and chronic hemodialysis-related amyloid), multiple sclerosis, and an MPS disorder (such as MPS I, MPS II, MPS IIIA, MPS IIIB, MPS IIIC, MPS HID, MPS IVA, MPS IVB, MPS VI, MPS VII, or MPS IX). In some embodiments, the diseases are autoimmune disorders (such as multiple sclerosis, rheumatoid arthritis, juvenile chronic arthritis, Ankylosing spondylitis, psoriasis, psoriatic arthritis, adult still disease, Becet syndrome, familial Mediterranean fever, Crohn's disease, leprosy, osteomyelitis, tuberculosis, chronic bronchiectasis, Castleman disease), or CNS disorders (such as spongiform encephalopathies (Creutzfeld-Jakob, Kuru, Mad Cow)). The compositions and methods of the disclosure can be used to treat individuals with lysosomal storage diseases comprising administering to a subject in need of treatment a therapeutically effective amount of a PIKfyve ASO or pharmaceutical composition described herein. In some embodiments, the ASOs and compositions of the disclosure decrease or inhibit the activity of PIKfyve and alters the biogenesis, function or dynamics of the endosomal or lysosomal systems in a way that reduces the abundance of the material abnormally stored in the lysosome in lysosomal storage diseases. In some embodiments, the ASOs and compositions target, decrease or inhibit the activity of PIKfyve thus altering the biogenesis, functions, or dynamics of the endoplasmic reticulum or Golgi apparatus in a way that reduces the abundance of the material abnormally stored in the lysosome in lysosomal storage diseases. In other embodiments, the disease is a neurological disorder.

As used herein, “sugar moiety” means an unmodified sugar moiety or a modified sugar moiety. The superscript prime symbol (′) is used to describe the numbering of a sugar in a nucleoside or nucleotide (the nucleobase positions are numbered without the prime). When describing the sugar only, the prime symbol is not used. As used herein, “unmodified sugar moiety” means a 2-OH(H) furanosyl moiety, as found in RNA (an “unmodified RNA sugar moiety”), or a 2-H(H) moiety, as found in DNA (an “unmodified DNA sugar moiety”). Unmodified sugar moieties have one hydrogen at each of the 1, 3, and 4 positions, an oxygen at the 3 position, and two hydrogens at the 5 position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2-substituted sugar moiety. Such modified furanosyl sugar moieties include bicyclic sugars and nonbicyclic sugars.

In certain embodiments, modified sugar moieties are nonbicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2, 4, and/or 5 positions. In certain embodiments one or more non-bridging substituent of nonbicyclic modified sugar moieties is branched. Examples of 2-substituent groups suitable for non-bicyclic modified sugar moieties include but are not limited to: 2-F, 2-OCH₃ (“OMe” or “O-methyl”), and 2-O(CH₂)₂OCH₃ (“MOE”). In certain embodiments, 2-substituent groups are selected from among: halo, allyl, amino, azido, SH, CN, OCN, CF₃, OCF₃, O-C₁₋₁₀ alkoxy, O-C₁₋₁₀ substituted alkoxy, O-C₁₋₁₀ alkyl, O-C₁₋₁₀ substituted alkyl, S-alkyl, N(Rₘ)-alkyl, O-alkenyl, S-alkenyl, N(Rₘ)-alkenyl, O-alkynyl, S-alkynyl, N(Rm)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁₋₁₀ alkyl, and the 2-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), and alkyl. Examples of 5-substituent groups suitable for non-bicyclic modified sugar moieties include but are not limited to: 5-methyl (R or S), 5-vinyl, and 5-methoxy. In certain embodiments, non-bicyclic modified sugar moieties comprise more than one non-bridging sugar substituent, for example, 2-F-5-methyl sugar moieties and the like.

In certain embodiments, a 2′-substituted non-bicyclic modified nucleoside comprises a sugar moiety comprising a nonbridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁₋₁₀ alkyl.

In certain embodiments, a 2′-substituted nucleoside nonbicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a nonbridging 2′-substituent group selected from: F, OCH₃, and OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4 and the 2 furanose ring atoms. Examples of such 4 to 2 bridging sugar substituents include but are not limited to: 4-CH₂-2, 4-(CH₂)₂-2, 4-(CH₂)₃-2, 4-CH₂—O-2 (“LNA”), 4-CH₂—-S-2, 4-(CH₂)₂—-O-2 (“ENA”), 4-CH(CH₃)—O-2 (referred to as “constrained ethyl” or “cEt”), 4-CH₂—O—CH₂-2, 4-CH₂—N(R)-2, 4-CH(CH₂OCH₃)—O-2 (“constrained MOE” or “cMOE”) and analogs thereof, 4-C(CH₃)(CH₃)—O-2 and analogs thereof, 4-CH₂—N(OCH₃)-2 and analogs thereof, 4-CH₂—O—N(CH₃)-2, 4-CH₂—C(H)(CH₃)-2, 4-CH₂—C(═CH₂)-2 and analogs thereof, 4-C(RₐR_{b})—N(R)—O-2, 4-C(RₐR_{b})—O—N(R)—2, 4-CH₂—O—N(R)-2, and 4-CH₂—N(R)—O-2, wherein each R, Rₐ, and R_{b}, is, independently, H, a protecting group, or C₁₋₁₂ alkyl.

In certain embodiments, such 4 to 2 bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently, H, a protecting group, hydroxyl, C₁₋₁₂ alkyl, substituted C₁₋₁₂ alkyl, C₁₋₁₂ alkenyl, substituted C₂₋₁₂ alkenyl, C₂₋₁₂ alkynyl, substituted C₂₋₁₂ alkynyl, C₅₋₂₀ aryl, substituted C₅₋₂₀ aryl, heterocycle radical, substituted heterocycle radical, heteroaryl, substituted heteroaryl, C₅₋₇ alicyclic radical, substituted C₅₋₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁₋₁₂ alkyl, substituted C₁₋₁₂ alkyl, C₂₋₁₂ alkenyl, substituted C₂₋₁₂ alkenyl, C₂₋₁₂ alkynyl, substituted C₂₋₁₂ alkynyl, C₅₋₂₀ aryl, substituted C₅₋₂₀ aryl, acyl (C(═O)—H), substituted acyl, a heterocycle radical, a substituted heterocycle radical, C₁₋₁₂ aminoalkyl, substituted C₁₋₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. In a preferred embodiment, the subject or patient is a human. The subject or patient may be undergoing other forms of treatment. In one embodiment, the patient has a neurological disease due to a mutation in the C9ORF72 gene (for instance, the patient may be haploinsufficient for the C9ORF72 gene (e.g., one which results in a 50% or greater reduction in C9ORF72 protein activity), or the C9ORF72 gene may comprises a GGGGCC repeat expansion (such as a (GGGGCC) n hexanucleotide expansion in C90RF72)). The variable “n” can be at least 30.

A “therapeutically effective amount,” or “effective dosage” or “effective amount” as used interchangeably herein unless otherwise defined, means a dosage of a drug effective for periods of time necessary, to achieve the desired therapeutic result. An effective dosage may be determined by a person skilled in the art and may vary according to factors such as the disease state, age, sex, and weight of the individual, and the ability of the drug to elicit a desired response in the individual. This term as used herein may also refer to an amount effective at bringing about a desired in vivo effect in an animal, mammal, or human, such as reducing and/or inhibiting the function of a receptor. A therapeutically effective amount may be administered in one or more administrations (e.g., the agent may be given as a preventative treatment or therapeutically at any stage of disease progression, before or after symptoms, and the like), applications or dosages and is not intended to be limited to a particular formulation, combination or administration route. It is within the scope of the present disclosure that the drug may be administered at various times during the course of treatment of the subject. The times of administration and dosages used will depend on several factors, such as the goal of treatment (e.g., treating v. preventing), condition of the subject, etc. and can be readily determined by one skilled in the art.

As used herein, the term “treat” or “treating” a subject, refers to administering a composition or agent described herein to the subject, such that at least one symptom of a disease or disorder is healed, alleviated, relieved, altered, remedied, reduced, ameliorated, or improved. Treating includes administering an amount effective to alleviate, relieve, alter, remedy, reduce, ameliorate, and/or improve one or more symptoms associated with a disease or disorder. The treatment may inhibit deterioration or worsening of a symptom associated with the disease or disorder.

The herein described methods of treatment may comprise administering to a subject in need thereof a composition comprising an effective amount of one or more antisense oligonucleotides that treats neurological diseases by inhibiting or suppressing PIKFYVE expression. The one or more antisense oligonucleotides may decrease or inhibit neurodegeneration. The one or more antisense oligonucleotides may decrease neuronal hyperexcitability. Reducing PIKFYVE mRNA and PIKFYVE protein levels suppresses neurodegeneration driven by toxic TDP-43 aggregates, DPR aggregates (for example, in C9ORF72-ALS patients), and by promoting the retention of TDP-43 in the nucleus. Delivery of the ASOs targeting PIKFYVE mRNA described herein reduces PIKFYVE protein levels.

The composition may inhibit kinase activity by inhibiting expression of a kinase. The composition may inhibit PIKFYVE kinase activity or expression. The one or more antisense oligonucleotides can be combined with small molecule therapeutic agents (such as apilimod and/or YM201636).

The disclosure provides oligonucleotides (modified or unmodified) that can be used to modulate PIKFYVE expression. Table 1 provides (5′ to 3′) generic sequence of bases for the PIKFYVE antisense oligonucleotides or inhibitory nucleic acids of the disclosure:

TABLE 1

| SEQ ID NO. | Sequence |
|---|---|
| 1 | ATGGCATGATCCCCATAAGC |
| 2 | AGGACCAGTACAACCTGTAG |
| 3 | AGGTGCCCCAACTTGTTACC |
| 4 | ACTAAGGTGCCCCAACTTGT |
| 5 | ACCAGGTAGTCCCAAATAAC |
| 6 | GCCTGACCCCTATACTTGAC |
| 7 | CTGACCCCTATACTTGACAG |
| 8 | TTAAGCCCTTTGGTCCAACC |
| 9 | GAGTTAGCACCCTCTACTAC |
| 10 | GTCATAAGTCCTTGGTCAAC |
| 11 | AAGCCTGACCCCTATACTTG |
| 12 | AGCCTGACCCCTATACTTGA |
| 13 | CATCCTATTAGCTTAAACCC |
| 14 | CCTGACCCCTATACTTGACA |
| 15 | TATGATCTGATAGCCAACTC |
| 16 | GTACCAGGACCAGTACAACC |
| 17 | CAGGACCAGTACAACCTGTA |
| 18 | ACACCCTTTGGAGTGTCTAG |
| 19 | GCCCCAACTTGTTACCTAAG |
| 20 | CCCCAATCTAGTTCAAGCAT |
| 21 | TCCAAGAGTTAGCACCCTCT |
| 22 | GGACCAGTACAACCTGTAGT |
| 23 | AAACCCAGTGTAGCTCCATT |
| 24 | TGCCCCAACTTGTTACCTAA |
| 25 | CCAATAGCAAGCAGCCTATA |
| 26 | CTACAATCAAATCCTGGTAG |
| 27 | GACCAAGTTTATGGACCCCA |
| 28 | GTGACCACCATCTATAGTTA |
| 29 | CTAAGGTAACTGTTCCTAGA |
| 30 | TGACCAAGTTTATGGACCCC |
| 31 | ACCAAGTTTATGGACCCCAA |
| 32 | GCAATAAAGCTAACCACATC |
| 33 | GATTCTACCACACAGTACAC |
| 34 | GAGCCCTAACTGTATCTTCC |
| 35 | TAGCTGATTGCCCCTTAACA |
| 36 | GAGACTGCTAAGGCTACTAC |
| 37 | GCTAAGCCCTAAGTATATCC |
| 38 | ACTACTGGTAAGGCAGTCCC |
| 39 | GGTATCCCTACACTTCTACA |
| 40 | GAGTACCAGGACCAGTACAA |
| 41 | ATAACCCCCCTGCTAAGAGC |
| 42 | CTTAGCTGATTGCCCCTTAA |
| 43 | GCTATACTACTAGAAGAACC |
| 44 | GTACCTAAATACAGGTCCTA |
| 45 | AGGTATCCCTACACTTCTAC |
| 46 | GCCTACATCCAGTTGATTAG |
| 47 | GAGTGGTAATCAGTCCTATT |
| 48 | GACAAAGTCCTACCTGGTAC |
| 49 | GTAATCTGTTGAGATACACC |
| 50 | TCCTTAATACCCCAGGTTAT |
| 51 | TTCCATAACTAAGGTGCCCC |
| 52 | AACTAAGGTGCCCCAACTTG |
| 53 | GTGCCCCAACTTGTTACCTA |
| 54 | CAAGTCCCTATAACATAACC |
| 55 | GAGCAGTCAGATGTAGTTCC |
| 56 | AAGGATGGTGTAGGTTCCAA |
| 57 | TAACCCACTAGAATAGCACC |
| 58 | GGTATGCCCACCAAAGTTGT |
| 59 | GTACACCTTAGTCTAACAGT |
| 60 | ACCAAGGTACTCTGAACCTA |
| 61 | TGAGTGGTAATCAGTCCTAT |
| 62 | TACCCTTACCTAATCAAGAG |
| 63 | GTGAGCCCTAACTGTATCTT |
| 64 | AATAACCCCCCTGCTAAGAG |
| 65 | AACCAATAGTGACTTATGGC |
| 66 | TTAGCTGATTGCCCCTTAAC |
| 67 | AGCTGATTGCCCCTTAACAG |
| 68 | GCTAGTTTACATACCTGTCC |
| 69 | CCTTAATACCCCAGGTTATC |
| 70 | TAACTAAGGTGCCCCAACTT |
| 71 | TAAGGTGCCCCAACTTGTTA |
| 72 | CCCAACTTGTTACCTAAGCA |
| 73 | GTTGGCACATCACACTATTA |
| 74 | ATGTTGAGGCTGTCACACTA |
| 75 | CTAACCTAAGGTAACTGTTC |
| 76 | AGGACTAGAGACCACTTAAC |
| 77 | ACTTAACCTCCCCCTACATC |
| 78 | ATGGTGTAGGTTCCAAACCC |
| 79 | GGATCTACTCCTACTACTCC |
| 80 | TGACCCCTATACTTGACAGA |
| 81 | CCTTTGGTCCAACCTATAAT |
| 82 | GGTAAAACCTGACCTATGTC |
| 83 | AGCCCAATAATGGAGTTGAC |
| 84 | GCCCAATAATGGAGTTGACA |
| 85 | TTACTGGACAGATCCCTTAC |
| 86 | CTGAGTGGTAATCAGTCCTA |
| 87 | AGAGTTAGCACCCTCTACTA |
| 88 | CCAATGCTGGTCAGTTGCTC |
| 89 | ACATAGTCCAATACCCTTAC |
| 90 | ACTGCCATTAGGACTGTTAC |
| 91 | GAAAGTAGTTGGACTCTCCC |
| 92 | TAGTTGGACTCTCCCAGTGC |
| 93 | GCTCCAAGTACTATGTCAAC |
| 94 | GGATAGAGGATTCAAGGCTC |
| 95 | TAACCCCCCTGCTAAGAGCC |
| 96 | ACTCAAGAACCAACCTGTAG |
| 97 | TGCTACAACCCAACTCCCCC |
| 98 | GATTGCCCCTTAACAGAACC |
| 99 | GTTTAGACTTGCCACACTAA |
| 100 | TAAGCCCTGTCAGCACAAGG |
| 101 | GGTCTTGTAGACACCAATAA |
| 102 | CTTAATACCCCAGGTTATCT |
| 103 | TTAATACCCCAGGTTATCTC |
| 104 | CATAACTAAGGTGCCCCAAC |
| 105 | AGTGGAGTACTATGGACTAA |
| 106 | GCCCATACTCAAGTTTATCC |
| 107 | CAATGTTGAGGCTGTCACAC |
| 108 | GGCATAGCTTATGGATCAAA |
| 109 | GGCTTTCACTCCACCAGATT |
| 110 | GACCCATCTCTCAGGTGATC |
| 111 | TAGCACAGGTATCCCTACAC |
| 112 | GCCTGTAGCCCTCCCCTAAA |
| 113 | TCCCAGTTGGTAATTCCACC |
| 114 | CCCAGTTGGTAATTCCACCC |
| 115 | TGGTAATTCCACCCCTCCAA |
| 116 | GGACTAGAGACCACTTAACC |
| 117 | GACCACTTAACCTCCCCCTA |
| 118 | GAGTACCCTCCAAAGAATTT |
| 119 | ATGGAAGCCTGACCCCTATA |
| 120 | GCCCTGAACTAGATAAACAC |
| 121 | GCCCTTTGGTCCAACCTATA |
| 122 | CCCTTTGGTCCAACCTATAA |
| 123 | GACAGTAAAGGCTCCACCTG |
| 124 | TCAACAAGAAGCCTACTGAC |
| 125 | ATCATAAGAGCCCCAATCAT |
| 126 | CATAAGAGCCCCAATCATCT |
| 127 | TGATCTGATAGCCAACTCAA |
| 128 | TCCTGAGTGGTAATCAGTCC |
| 129 | GACCTACTACAAACTATCAG |
| 130 | AGACCAGGTTCAATAGAATC |
| 131 | AGTACCAGGACCAGTACAAC |
| 132 | AGTCCAATACCCTTACCTAA |
| 133 | GCATGTTAGATCCAAATCCC |
| 134 | GCCCAATAGCAAGCAGCCTA |
| 135 | GACTGACTAAAGGAGGAGCC |
| 136 | CAACCAGTATCAATACCCTC |
| 137 | TGGATAGAGGATTCAAGGCT |
| 138 | GTCCCAGCAAATCAACTACC |
| 139 | GTAGTTACCACCTAAACAGA |
| 140 | GATCACCAGACTCAAGAACC |
| 141 | GCAGCAACCTACCTGACATA |
| 142 | AGCAACCTACCTGACATACC |
| 143 | GCAACCTACCTGACATACCT |
| 144 | GACACCCTTTGGAGTGTCTA |
| 145 | CCTTAGCTGATTGCCCCTTA |
| 146 | TACCATATAGGAAACCTCCT |
| 147 | CTATACTACTAGAAGAACCC |
| 148 | GTGCCCCCAAACAAGAAGTT |
| 149 | GTACACAAACATCCCTCTAA |
| 150 | CCATATTACACCTTAGGAAC |
| 151 | CCCCAACTTGTTACCTAAGC |
| 152 | CCAACTTGTTACCTAAGCAC |
| 153 | GCCTAACCAGTGGAGTACTA |
| 154 | ACCAGTGGAGTACTATGGAC |
| 155 | GTATACCATCCACCTGAGTT |
| 156 | GTGGCAAAAGTTCACTACTC |
| 157 | GGTACTCTTCAGATACCTAA |
| 158 | GTACCTGAATCAAGACCCAC |
| 159 | TGGTAAGGCAGTCCCAACAA |
| 160 | ACAGGCATAGCTTATGGATC |
| 161 | AGGCATAGCTTATGGATCAA |
| 162 | TAGCACCCTAAACCATCAAT |
| 163 | AGCAAGTAGTTACCCTTGAG |
| 164 | TCCACTATTAAGGATCTTCC |
| 165 | GCTGCCACCTAGAATTAGGT |
| 166 | TTAGCACAGGTATCCCTACA |
| 167 | TAGCCTGTAGCCCTCCCCTA |
| 168 | GTTGGTAATTCCACCCCTCC |
| 169 | CCTACATCCAGTTGATTAGC |
| 170 | GATTCACAGGACTAGAGACC |
| 171 | AGGTGTCCAAACTACCATAA |
| 172 | GTGTCCAAACTACCATAAAC |
| 173 | CCTAACCAGTCAGCTATTAG |
| 174 | GATCCCTTTCCATGTACTAG |
| 175 | GCCTAGCATATATTACCCCA |
| 176 | GGAGACCACTATATTATCCC |
| 177 | GACAACCAGCAGAATCCCTA |
| 178 | GGTACTATATCCAACTGGAC |
| 179 | CATTAGGTATGGCATGATCC |
| 180 | GAACATCACTTAAATGGTCC |
| 181 | AGCCCTTTGGTCCAACCTAT |
| 182 | GTATGCCCACCAAAGTTGTC |
| 183 | ATACCAATGGCAAGGTTTGG |
| 184 | GCAAGTGGCAGCCCAATAAT |
| 185 | GCCAATAATCACACCCTTGG |
| 186 | ACAGATCCCTTACACTATCA |
| 187 | ATCCCATACAGCCAGTTTGG |
| 188 | GCAACCAAGGTACTCTGAAC |
| 189 | TAAGTCCTTGGTCAACTTGC |
| 190 | GATGCCACCTAAATTGCTGG |
| 191 | GGATTCCCAGTTTAAGTCAA |
| 192 | GAAAGGTTATCTGTTGTGCC |
| 193 | GACTCCTTATAGTCACTACC |
| 194 | TACCAGGACCAGTACAACCT |
| 195 | GACCAGTACAACCTGTAGTA |
| 196 | GGTACAAAAGGTTCCAGTAG |
| 197 | GTACAAAAGGTTCCAGTAGC |
| 198 | GCTTCACATAGTCCAATACC |
| 199 | GTCCAATACCCTTACCTAAT |
| 200 | TACCTAATCAAGAGAAGGTC |
| 201 | TGAAAGTAGTTGGACTCTCC |
| 202 | AAAGTAGTTGGACTCTCCCA |
| 203 | ACCTAAGCCCACCTACAATA |
| 204 | ACTAAGTTCAGCTACCACCA |
| 205 | AACCCCCCTGCTAAGAGCCA |
| 206 | GCACTGTGGCTATTACACCC |
| 207 | ACCATGATGCTACCCTCAGT |
| 208 | GCCCCTTGAGTGCTGTTTAT |
| 209 | ACCAGGAGCATTTGTTGATC |
| 210 | ATGCTACAACCCAACTCCCC |
| 211 | GCTGATTGCCCCTTAACAGA |
| 212 | GACTGCTAAGGCTACTACAA |
| 213 | GTGAGGATCATGTAACAGTC |
| 214 | AGTTTAGACTTGCCACACTA |
| 215 | GTGTACCTAAATACAGGTCC |
| 216 | TGCCCACTGAAACCTTACTC |
| 217 | TATGCCCATCCCTAAGTTGT |
| 218 | GGACTATCTCTAATCAGTGG |
| 219 | AAGGTGCCCCAACTTGTTAC |
| 220 | TCATTGCCTTACCTAAGTAC |
| 221 | CATTGCCTTACCTAAGTACA |
| 222 | CAGTGGAGTACTATGGACTA |
| 223 | GGAGTACTATGGACTAAGAA |
| 224 | TAAGCCCTAAGTATATCCTC |
| 225 | AAGTTCACTACTCCCAACTA |
| 226 | GGTTGGCACATCACACTATT |
| 227 | GTTTGCTTAACCAATGCTGG |
| 228 | ACAGTTTGCCTAAACCTGGC |
| 229 | ACACAATCCCATGATAGGAC |
| 230 | CCCCCAATCTAGTTCAAGCA |
| 231 | GACTACCTCCTACTTTTAGT |
| 232 | AAGTACCTGAATCAAGACCC |
| 233 | CTACTGGTAAGGCAGTCCCA |
| 234 | GGTAAGGCAGTCCCAACAAA |
| 235 | TTAACAGCAAGTAGTTACCC |
| 236 | TCACACTAACCTAAGGTAAC |
| 237 | GATCTAAGAGTTAAGCTCTC |
| 238 | TAGAATACTTGACCCATCTC |
| 239 | GCAACCCTATGTAAGTCTAT |
| 240 | GGCTGCCACCTAGAATTAGG |
| 241 | GTATCCCTACACTTCTACAC |
| 242 | AACTGTTCCTCCCAGTTGGT |
| 243 | CCAGTTGGTAATTCCACCCC |
| 244 | TTGGTAATTCCACCCCTCCA |
| 245 | GGTCAAAGACCTGAGTCACC |
| 246 | ACTCATGGAGTATTACTGCC |
| 247 | ATGGAGTATTACTGCCCCAA |
| 248 | AACCTCCCCCTACATCCTAT |
| 249 | CCCATCACATCAAGTTACAG |
| 250 | TATGACAATCAATCCCACCC |
| 251 | GAAATTCCCCTACCCAGTCC |
| 252 | AATTCCCCTACCCAGTCCTA |
| 253 | CCCTAGACAGTGTAGTAGTT |
| 254 | TTCCAAGAGTTAGCACCCTC |
| 255 | GTATATCCCAATGATACCAG |
| 256 | AACCAGTCAGCTATTAGAAC |
| 257 | GCCCTGCCATCAAAAAACTC |
| 258 | TCAGGATCTACTCCTACTAC |
| 259 | CAGGATCTACTCCTACTACT |
| 260 | GAGCCACTTACAGATGATCC |
| 261 | GTTATTAGACACCTACTCTC |
| 262 | CTCTGTAGTAGTTTAGGTGG |
| 263 | GGAAGCCTGACCCCTATACT |
| 264 | GACCCCTATACTTGACAGAA |
| 265 | CTGCCCTGAACTAGATAAAC |
| 266 | GCTAAAACTCCAATCCTATC |
| 267 | CCTATTAGCTTAAACCCATC |
| 268 | ACCATTTGCTAGATAGGTGC |
| 269 | GGCAGCCCAATAATGGAGTT |
| 270 | GCAGCCCAATAATGGAGTTG |
| 271 | ACAACTATGATCTGATAGCC |
| 272 | GGAAGCTAGTTATACAACAC |
| 273 | ATCAGTCCTATTAACCTACC |
| 274 | GGTTAACTTAGCTTGGTCTC |
| 275 | GACAGGATTCCCAGTTTAAG |
| 276 | ACTTGAGTACCAGGACCAGT |
| 277 | CTTCACATAGTCCAATACCC |
| 278 | TTCACATAGTCCAATACCCT |
| 279 | CATAGTCCAATACCCTTACC |
| 280 | TAGTCCAATACCCTTACCTA |
| 281 | GGCTGCTCAATGACAAGTGG |
| 282 | GGACTAACCCAGAGGTCACC |
| 283 | GAGTCTGCCTATTCCTGATC |
| 284 | AGTAGTTGGACTCTCCCAGT |
| 285 | GTTCCTACAGTTTAACACAG |
| 286 | GGTTCCCCACCAACAGAATG |
| 287 | TACCTAAGCCCACCTACAAT |
| 288 | ACCAGTATCAATACCCTCAA |
| 289 | GATGATCTCAGCTAGAATCC |
| 290 | GATCTCAGCTAGAATCCTTA |
| 291 | ATGGATAGAGGATTCAAGGC |
| 292 | AGCCCTAACTGTATCTTCCC |
| 293 | CTAAGAGTGATGACAGTTCC |
| 294 | GGACACTTAAACAGGCACTA |
| 295 | GAAAAATAACCCCCCTGCTA |
| 296 | AAAATAACCCCCCTGCTAAG |
| 297 | GACTGACTCCTATCCAACAC |
| 298 | CACCCTATTATACTCAGAGC |
| 299 | GTTTCTAGCCCCTTGAGTGC |
| 300 | GTACCATATAGGAAACCTCC |
| 301 | ACCATATAGGAAACCTCCTC |
| 302 | CAAGTTTAGACTTGCCACAC |
| 303 | CCTGCTGAAGCTATACTACT |
| 304 | TACTAGAAGAACCCATGAGC |
| 305 | GATCCAGGATTATCATACCA |
| 306 | GCTTCACCCTTCTAGGACTA |
| 307 | GATTGCTCCTACCACTCTTG |
| 308 | GGTTACTTCACAAACTCCAA |
| 309 | GCTGACCAAGTTTATGGACC |
| 310 | GGTATTACACACTCAGCCTA |
| 311 | GTATTACACACTCAGCCTAG |
| 312 | GTCTCCTTAATACCCCAGGT |
| 313 | CTTCCATAACTAAGGTGCCC |
| 314 | GGTGCCCCAACTTGTTACCT |
| 315 | CCCCTGTTCTCTAATGTACT |
| 316 | GCACTGCCAAGCTATCAGAT |
| 317 | TGCCAAGCTATCAGATAAGC |
| 318 | CCAGTGGAGTACTATGGACT |
| 319 | CTAATCAATGTGCTAAGCCC |
| 320 | GCACAGAATTAGCCCATACT |
| 321 | GAATTAGCCCATACTCAAGT |
| 322 | CTTAGAAGTCCCCAAGTCTA |
| 323 | CCTCAATAACCATGACAGGT |
| 324 | TCAGGTACTCTTCAGATACC |
| 325 | GCTCTTACATTCACCAGATA |
| 326 | TTGCTTAACCAATGCTGGTG |
| 327 | GCATACAGTTTGCCTAAACC |
| 328 | CACAATCCCATGATAGGACT |
| 329 | CAATCCCATGATAGGACTAT |
| 330 | TGTCAACCTAACAAGTTGGT |
| 331 | GTCAACCTAACAAGTTGGTT |
| 332 | TACTGGTAAGGCAGTCCCAA |
| 333 | CACCAGGTAGTCCCAAATAA |
| 334 | GGTAGTCCCAAATAACTTTC |
| 335 | ATTAGCACAGGTATCCCTAC |
| 336 | GTAGCCCTCCCCTAAATTCT |
| 337 | GGTAATTCCACCCCTCCAAC |
| 338 | GTCAAAGACCTGAGTCACCT |
| 339 | TGTGAGAGATCAACTCAACA |
| 340 | GGAGTATTACTGCCCCAAAA |
| 341 | CTAGAGACCACTTAACCTCC |
| 342 | GAGACCACTTAACCTCCCCC |
| 343 | CCCCCTACATCCTATCACTA |
| 344 | CTATAGTGTATCCAGCCAAT |
| 345 | GGATGGTGTAGGTTCCAAAC |
| 346 | GATGGTGTAGGTTCCAAACC |
| 347 | TGTTACCACATTTCAGTCCC |
| 348 | AACTTCCAAGAGTTAGCACC |
| 349 | AGTACCCTCCAAAGAATTTC |
| 350 | GCCTAACCAGTCAGCTATTA |
| 351 | CCAGTCAGCTATTAGAACTA |
| 352 | GGTTTTGTCAAGCTAACAAC |
| 353 | GGCTGTAATGACCTCCCCTG |
| 354 | CTCAGGATCTACTCCTACTA |
| 355 | AGGATCTACTCCTACTACTC |
| 356 | GAAGCCTGACCCCTATACTT |
| 357 | GTAAAACCTGACCTATGTCC |
| 358 | GCTTAAACCCATCAAACTGA |
| 359 | TCCAAATGGCTGATGTAACC |
| 360 | CAGCCCAATAATGGAGTTGA |
| 361 | GGTCTGTTTACTGGACAGAT |
| 362 | CAGATCCCTTACACTATCAA |
| 363 | ATCCAGACCTTTAGTTACCA |
| 364 | AATCATAAGAGCCCCAATCA |
| 365 | GATCTGATAGCCAACTCAAT |
| 366 | GGATGGCAAGATCCCATACA |
| 367 | GAACCCCAAGAGTGATGACC |
| 368 | CCTGAGTGGTAATCAGTCCT |
| 369 | TAACCTACCTACCCAAAGCC |
| 370 | GCACTAGATGGTTTGAATAC |
| 371 | GATCAGGCTCATTCACAACA |
| 372 | GCCACAAAGTTACCTACTAG |
| 373 | GGAACTTAGCATCACATTAG |
| 374 | GACAGCTCCACAGGTATCAA |
| 375 | GCTATTATTAGACCAGGTTC |
| 376 | GACTTGAGTACCAGGACCAG |
| 377 | TTGAGTACCAGGACCAGTAC |
| 378 | CAGTTCAAGGACTAGCTCTT |
| 379 | CACATAGTCCAATACCCTTA |
| 380 | ATAGTCCAATACCCTTACCT |
| 381 | CTGCCATTAGGACTGTTACA |
| 382 | TGCCATTAGGACTGTTACAG |
| 383 | AAGGCTCACCTATAAACAAC |
| 384 | GTATGACCATCCTTACATAC |
| 385 | AAGTAGTTGGACTCTCCCAG |
| 386 | AGTTGGACTCTCCCAGTGCC |
| 387 | GTACAGGTCACTACAGATGA |
| 388 | TAATATCCACTCCTACCTGG |
| 389 | CCTACCTAAGCCCACCTACA |
| 390 | GTGTAGCTCCATTTCAACTT |
| 391 | GGTGCTACTTACTGAACACC |
| 392 | CCCTAACTGTATCTTCCCAA |
| 393 | CACTGGCTAGTGCTTTATCC |
| 394 | GCAACTGTCAACCAAGACTG |
| 395 | GTAATCCAGGTCATAGTTCC |
| 396 | CCAATAGTGACTTATGGCAT |
| 397 | CCAGGCAGCAACCTACCTGA |
| 398 | GGAGACTGCTAAGGCTACTA |
| 399 | AGACTGCTAAGGCTACTACA |
| 400 | GCATTACCAATAAATCCACC |
| 401 | TGGTCAGTCTACCCAGTTAA |
| 402 | GCCCTTCTCACATTAAGCAT |
| 403 | AATTACCATGCCCCAGAGAC |
| 404 | CCTAAATACAGGTCCTAATG |
| 405 | AACATCAATCTGAGACACCC |
| 406 | GTTGCCTATCCTCACTTACC |
| 407 | CCTCAGGTCAATCTATGCTA |
| 408 | TATGCCCACTGAAACCTTAC |
| 409 | AGGTTACTTCACAAACTCCA |
| 410 | GCCCATATTACACCTTAGGA |
| 411 | ATAACTAAGGTGCCCCAACT |
| 412 | ATCTTCCACTAAGAAGTCCC |
| 413 | TGCTAAGCCCTAAGTATATC |
| 414 | AAGCCCTAAGTATATCCTCA |
| 415 | GTATCTATATGAGACTCCAG |
| 416 | TATACCATCCACCTGAGTTC |
| 417 | GCAAAAGTTCACTACTCCCA |
| 418 | GTTCACTACTCCCAACTACT |
| 419 | AGGTACTCTTCAGATACCTA |
| 420 | GTCCCTATAACATAACCTAG |
| 421 | CAAGTGACCACCATCTATAG |
| 422 | GCTAGTCAAGTCTCATTAAC |
| 423 | TTACACAATCCCATGATAGG |
| 424 | ACAATCCCATGATAGGACTA |
| 425 | CCCAATCTAGTTCAAGCATC |
| 426 | AGATGGTGACTACCTCCTAC |
| 427 | CTGGTAAGGCAGTCCCAACA |
| 428 | TAAGGCAGTCCCAACAAAAC |
| 429 | AAGCAGGACCTGTCTGTTAC |
| 430 | GACAGGCATAGCTTATGGAT |
| 431 | CTAGATTAGTGAACAGTAGC |
| 432 | CACACTAACCTAAGGTAACT |
| 433 | CCAGGTAGTCCCAAATAACT |
| 434 | GTAGTCCCAAATAACTTTCC |
| 435 | ATTGCCCCCAGACTCCTACT |
| 436 | CCCCAGACTCCTACTTAAAC |
| 437 | GAGTATAGAATACTTGACCC |
| 438 | GTAAATTAGCACAGGTATCC |
| 439 | ACTGTTCCTCCCAGTTGGTA |
| 440 | GTTCCTCCCAGTTGGTAATT |
| 441 | CTCCCAGTTGGTAATTCCAC |
| 442 | CAGTTGGTAATTCCACCCCT |
| 443 | AGTTGGTAATTCCACCCCTC |
| 444 | GTACCCTGATGATACCCTTC |
| 445 | GGTTAGAATGGATCAAAACC |
| 446 | GTCAAAGTAGGTCCACTGAA |
| 447 | ACTTCCAAGAGTTAGCACCC |
| 448 | ACCCTCTACTACCAAGCAGT |
| 449 | GGTGTCCAAACTACCATAAA |
| 450 | CTAACCAGTCAGCTATTAGA |
| 451 | ATCAGCAAGAGCCTATTAAG |
| 452 | GCAGGTTCAGTGCTAAGATT |
| 453 | GTAAGACTTGCTGACTCACC |
| 454 | CAAGTAGCTGTAAGTCCAAC |
| 455 | ATCCCCAAAGAGACCTATGC |
| 456 | CAACCAGCAGAATCCCTATA |
| 457 | TTAGGTATGGCATGATCCCC |
| 458 | TAAGCCCTTTGGTCCAACCT |
| 459 | AAGCCCTTTGGTCCAACCTA |
| 460 | TACCAATGGCAAGGTTTGGC |
| 461 | TTGTTCTGGCTGACCAAGTC |
| 462 | GTTCTGGCTGACCAAGTCTG |
| 463 | TCACCTTGACTTAGGCAAAC |
| 464 | GGCAAACCACTCCAAGTTGA |
| 465 | CAAGTGGCAGCCCAATAATG |
| 466 | TTTACTGGACAGATCCCTTA |
| 467 | TACTGGACAGATCCCTTACA |
| 468 | GGACAGATCCCTTACACTAT |
| 469 | CTGAACAGAGTACTAACCAC |
| 470 | ATAAGAGCCCCAATCATCTA |
| 471 | CCTAAACACATGAACCTGGT |
| 472 | TAATGTAGACTGGTCCAGGC |
| 473 | AAGGAGCCCCCAGTTGATCT |
| 474 | GATCCCATACAGCCAGTTTG |
| 475 | TTGAGGCAACCAAGGTACTC |
| 476 | CAACCAAGGTACTCTGAACC |
| 477 | CCAAGGTACTCTGAACCTAA |
| 478 | AGTAGAACTGTCACTATACC |
| 479 | GTAACAACCACCCCTGGAAA |
| 480 | TTCCTGAGTGGTAATCAGTC |
| 481 | GACTACCTCTATTGTCAACA |
| 482 | ACACCTTGTACTTCAAGTGC |
| 483 | CTTAGTAGTCAGAACTAGCC |
| 484 | GGACAGTCTAAACAGCCACA |
| 485 | GGAAAGGTTATCTGTTGTGC |
| 486 | AAGGTTATCTGTTGTGCCAC |
| 487 | GGACAAAGTCCTACCTGGTA |
| 488 | TGAGTACCAGGACCAGTACA |
| 489 | ACCCTTACCTAATCAAGAGA |
| 490 | TAAAGGAAGCATCCCCTTGT |
| 491 | AACTGCCATTAGGACTGTTA |
| 492 | TGAGAAAGGCTCACCTATAA |
| 493 | ATCTATGCCTTCCAAGGATC |
| 494 | GCTACAGCCCAATAGCAAGC |
| 495 | GGTCATCAGAGTCTGCCTAT |
| 496 | CCTGATCTAAACCCATACCC |
| 497 | GTAGTTGGACTCTCCCAGTG |
| 498 | ACAGACACTAGTATTGCCTC |
| 499 | AATATCCACTCCTACCTGGT |
| 500 | TGGTTCCCCACCAACAGAAT |

In one embodiment, the disclosure provides modified oligonucleotides consisting of 12-30 linked nucleosides and having a nucleobase sequence comprising at least 8, at least 9, at least 10, at least 11 at least 12, at least 13, at least 14, at least 15, at least 16, at least 17, at least 18, at least 19 or at least 20 consecutive nucleotide bases of any of the nucleobase sequences of SEQ ID NO:1-500 in Table 1. In some embodiments, the modified oligonucleotide is at least 80% to 100% (i.e., 80%, 82%, 84%, 86%, 88%, 90%, 92%, 94%, 96%, 98% or 100%; or any numerical range or value between any of the foregoing values) identical to any of the sequences comprising or consisting of SEQ ID NO:1-500.

The sequences provided in Table 1 can be used to design antisense molecules for inhibition of PIKFYVE expression. For example, gapmer oligonucleotides can be designed using the sequences in Table 1 and can comprise a 5′-wing of about 3-5 nucleotides, a 3′-wing of about 3-5 nucleotides and a gap region comprising 8-12 consecutive deoxyribonucleosides of any one of the sequences of Table 1. In one embodiment, an oligonucleotide of the disclosure comprises a gapmer having a gap segment of at least 8, at least 9, at least 10, at least 11 at least 12, at least 13, at least 14, at least 15, at least 16, at least 17, at least 18, at least 19 or at least 20 consecutive nucleotide bases of any of the nucleobase sequences of SEQ ID NO:1-136 in Table 1; flanked by a 5′ and 3′ wing segments, wherein the gap segment is located between the 5′ and 3′ wing segments and wherein each of the wing segments comprises a modified sugar. In one embodiment, the gap segment is 8-10 nucleosides in length and each wing segment is 3-5 modified nucleosides in length. In yet another embodiment, an oligonucleotide of the disclosure comprises a 5′ wing segment comprising modified sugars and having the nucleobase sequence of the first 3-5 nucleobases of any of SEQ ID NO:1-500, followed by a gap of the next 8-12 unmodified nucleotides of the same sequence corresponding to SEQ ID NO:1-500, followed by a 3′ wing segment comprising modified sugars and having the nucleobase sequence of the last 3-5 nucleobases of the same sequence corresponding to SEQ ID NO:1-500. Table 2 provides MOE gapmers of the disclosure.

The 5′ and/or 3′ wings can comprise the following chemistries: 2′-OMe, 2′-MOE, LNA or DNA, by themselves or used in combination with one another. The backbone linkage of the 5′ and/or 3′ wings can be phosphorothioate or a mixture of phosphodiester and phosphorothioate. Linkages in the gap region can be phosphorothioate.

In some embodiments, the oligonucleotide is single stranded. In some embodiments the oligonucleotide comprises or is complexed with a moiety that neutralizes charge on the oligonucleotide to promote uptake and transfer across a cell membrane.

In one embodiment, each of the ASOs in Table 1 has the following 5-10-5 motif: 2MOE*2MOE-2MOE-2MOE-2MOE-N*N*N*N*N*N*N*N*N*N*2MOE-2MOE-2MOE*2MOE*2MOE where (i) 2MOE is a nucleobase with a 2′-OCH₂CH₂—OCH₃ group (i.e., 2′-MOE), (ii) N is a nucleobase, (iii) the asterisk (*) refers to a phosphorothioate linkage, and (iv) the dash (-) refers to a phosphodiester linkage. Table 2 below shows this motif on SEQ ID NO: 1-33 (here SEQ ID NO: 501-533).

Table 2: The Sequence of Bases in PIKFYVE Antisense Oligonucleotides-(ASOs). (Gapmer design: 5′-five 2′-methoxyethylribose nucleotides-ten DNA nucleotides-five 2′-methoxyethylribose nucleotides-3′; Capital letters are 2′-methoxyethylribose nucleosides; lower case are DNA nucleosides; asterisks (*) are phosphorothioate linkages; linkages which do not have an asterisk are phosphodiester linkages) (Note that the following table provides 2′MOE wings; however, alternative wings comprising 2′-OMe or LNA (locked nucleic acid) are contemplated).

TABLE 2

| SEQ ID | |
|---|---|
| NO. | Sequence |
| 501 | A*TGGCa*t*g*a*t*c*c*c*c*a*TAA*G*C |
| 502 | A*GGACc*a*g*t*a*c*a*a*c*c*TGT*A*G |
| 503 | A*GGTGc*c*c*c*a*a*c*t*t*g*TTA*C*C |
| 504 | A*CTAAg*g*t*g*c*c*c*c*a*a*CTT*G*T |
| 505 | A*CCAGg*t*a*g*t*c*c*c*a*a*ATA*A*C |
| 506 | G*CCTGa*c*c*c*c*t*a*t*a*c*TTG*A*C |
| 507 | C*TGACc*c*c*t*a*t*a*c*t*t*GAC*A*G |
| 508 | T*TAAGc*c*c*t*t*t*g*g*t*c*CAA*C*C |
| 509 | G*AGTTa*g*c*a*c*c*c*t*c*t*ACT*A*C |
| 510 | G*TCATa*a*g*t*c*c*t*t*g*g*TCA*A*C |
| 511 | A*AGCCt*g*a*c*c*c*c*t*a*t*ACT*T*G |
| 512 | A*GCCTg*a*c*c*c*c*t*a*t*a*CTT*G*A |
| 513 | C*ATCCt*a*t*t*a*g*c*t*t*a*AAC*C*C |
| 514 | C*CTGAc*c*c*c*t*a*t*a*c*t*TGA*C*A |
| 515 | T*ATGAt*c*t*g*a*t*a*g*c*c*AAC*I*C |
| 516 | G*TACCa*g*g*a*c*c*a*g*t*a*CAA*C*C |
| 517 | C*AGGAc*c*a*g*t*a*c*a*a*c*CTG*T*A |
| 518 | A*CACCc*t*t*t*g*g*a*g*t*g*TCI*A*G |
| 519 | G*CCCCa*a*c*t*t*g*t*t*a*c*CTA*A*G |
| 520 | C*CCCAa*t*c*t*a*g*t*t*c*a*AGC*A*T |
| 521 | T*CCAAg*a*g*t*t*a*g*c*a*c*CCI*C*T |
| 522 | G*GACCa*g*t*a*c*a*a*c*c*t*GTA*G*T |
| 523 | A*AACCc*a*g*t*g*t*a*g*c*t*CCA*T*T |
| 524 | T*GCCCc*a*a*c*t*t*g*t*t*a*CCT*A*A |
| 525 | C*CAATa*g*c*a*a*g*c*a*g*c*CTA*T*A |
| 526 | C*TACAa*t*c*a*a*a*t*c*c*t*GGT*A*G |
| 527 | G*ACCAa*g*t*t*t*a*t*g*g*a*CCC*C*A |
| 528 | G*TGACc*a*c*c*a*t*c*t*a*t*AGT*T*A |
| 529 | C*TAAGg*t*a*a*c*t*g*t*t*c*CTA*G*A |
| 530 | T*GACCa*a*g*t*t*t*a*t*g*g*ACC*C*C |
| 531 | A*CCAAg*t*t*t*a*t*g*g*a*c*CCC*A*A |
| 532 | G*CAATa*a*a*g*c*t*a*a*c*c*ACA*T*C |
| 533 | G*ATTCt*a*c*c*a*c*a*c*a*g*TAC*A*C |

The PIKFYVE kinase antisense or inhibitory nucleic acids of the disclosure can inhibit the expression and thus the activity associate with PIKFYVE. The PIKFYVE kinase antisense or inhibitory nucleic acids can include any combination of the oligonucleotides set forth in Table 2 and sequences that are 98%-99% identical thereto.

The PIKFYVE ASOs described herein, such as SEQ ID NO:501-533, suppress PIKFYVE mRNA expression with minimal off-target binding.

Methods of treatment may include any number of modes of administering a disclosed composition. Modes of administration may include aqueous, lipid, oily or other solutions, solutions in simulated cerebrospinal fluid, emulsions such as oil-in-water emulsions, liposomes, aqueous or oily suspensions and the like. Typically, an ASO of the disclosure will be administered directly to the CNS of the subject. Accordingly, the formulation or composition will be sterile and more preferably be suitable for injection. The following formulations and methods are merely exemplary and are in no way limiting.

Formulations suitable for parenteral administration include aqueous and non-aqueous, isotonic sterile injection solutions, which may contain anti-oxidants, buffers, bacteriostats, and solutes that render the formulation isotonic with the blood of the intended recipient, and aqueous and non-aqueous sterile suspensions that may include suspending agents, solubilizers, thickening agents, stabilizers, and preservatives. The formulations may be presented in unit-dose or multi-dose sealed containers, such as ampules and vials, and may be stored as liquids or in a freeze-dried (lyophilized) condition requiring only the addition of the sterile liquid excipient, for example, water for injections, immediately prior to use. Extemporaneous injection solutions and suspensions may be prepared from sterile powders, granules, and tablets. The formulation may be provided in a pre-filled syringe.

Additional therapeutic agent(s) may be administered simultaneously or sequentially with the disclosed one or more antisense or inhibitory nucleic acids and compositions. Sequential administration includes administration before or after the disclosed one or more antisense or inhibitory nucleic acids or compositions. In some embodiments, the additional therapeutic agent or agents may be administered in the same composition as the disclosed one or more antisense or inhibitory nucleic acids. In other embodiments, there may be an interval of time between administration of the additional therapeutic agent and the disclosed one or more antisense or inhibitory nucleic acids. In some embodiments, administration of an additional therapeutic agent with a disclosed one or more antisense or inhibitory nucleic acids may allow lower doses of the other therapeutic agents and/or administration at less frequent intervals. When used in combination with one or more other active ingredients, the one or more antisense or inhibitory nucleic acids of the disclosure and the other active ingredients may be used in lower doses than when each is used singly. Accordingly, the pharmaceutical compositions of the disclosure include those that contain one or more other active ingredients, in addition to one or more antisense or inhibitory nucleic acids of the disclosure. The above combinations include combinations of one or more antisense or inhibitory nucleic acids of the disclosure not only with one other active compound, but also with two or more other active compounds. For example, the compound of the disclosure may be combined with a variety of drugs to treat neurological diseases. The antisense oligonucleotide may be covalently linked to another oligonucleotide, such as one with a target other than PIKFYVE. The antisense oligonucleotide may be covalently linked to an antibody.

The disclosed one or more antisense or inhibitory nucleic acids can be combined with the following, but are not limited, anticholinergic drugs, anticonvulsants, antidepressants, benzodiazepines, decongestants, muscle relaxants, pain medications, and/or stimulants. Additional types of therapy and treatment include, but are not limited to digital communication devices, feeding tubes, mechanical ventilation, nutritional support, deep brain stimulation, occupational therapy, physical therapy, and/or speech therapy.

The disclosed composition(s) may be incorporated into a pharmaceutical composition suitable for administration to a subject (such as a patient, which may be a human or non-human). The pharmaceutical compositions may comprise a carrier (e.g., a pharmaceutically acceptable carrier). Any suitable carrier can be used within the context of the disclosure, and such carriers are well known in the art. The choice of carrier will be determined, in part, by the particular use of the composition (e.g., administration to an animal) and the particular method used to administer the composition. Accordingly, there is a wide variety of suitable formulations of the composition of the present invention.

The pharmaceutical compositions may include a therapeutically effective amount or a prophylactically effective amount of the antisense oligonucleotide. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of one or more antisense or inhibitory nucleic acids of the disclosure are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions may include one or more pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogenfree water; isotonic saline; Ringer's solution; ethyl alcohol, and phosphate buffer solutions, as well as other non-toxic compatible lubricants such as, but not limited to, sodium lauryl sulfate and magnesium stearate, as well as releasing agents, coating agents, preservatives and antioxidants may also be present in the composition, according to the judgment of the formulator.

The route by which the disclosed one or more antisense or inhibitory nucleic acids are administered, and the form of the composition will dictate the type of carrier to be used.

The pharmaceutical compositions of the disclosure can be administered in a number of ways depending upon whether local or systemic treatment is desired and upon the area to be treated. Administration can be parenteral including intravenous, intraarterial, subcutaneous, intraperitoneal or intramuscular injection or infusion; or intracranial, e.g., intrathecal, intracerebroventricular, or intraventricular, administration. In one embodiment the antisense or inhibitory nucleic acid is administered by intravenous, intraperitoneal, or as a bolus injection or administered directly into the target organ. In another embodiment, the antisense or inhibitory nucleic acid is administered intrathecally or intra-cerebroventricular as a bolus injection.

Carriers for systemic administration typically include at least one of solvent, diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmelose, crospovidone, sodium carboxymethyl starch, sodium starch glycolate, clays, and ion exchange resins. The amount of disintegrant(s) in a systemic composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include A VICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and Mccutcheon's Volume 1, Emulsifiers & Detergents, 1994, North American Edition, pp. 236-239. The amount of surfactant(s) in the systemic or topical composition is typically about 0.1% to about 5%.

Compositions and formulations for parenteral, intrathecal, intra-cerebroventricular, or intraventricular administration can include sterile aqueous solutions which can also contain buffers, diluents and other suitable additives such as, but not limited to, penetration enhancers, carrier compounds and other pharmaceutically acceptable carriers or excipients. For example, an intrathecal cerebrospinal fluid (CSF) catheter can be used to deliver antisense formulations of the disclosure. The catheter can be inserted at the L3 or L4 vertebrae. The distal tip of the catheter extends within the intrathecal space to approximately the L1 vertebrae. Antisense oligonucleotides are dissolved in saline, are sterilized by filtration, and are administered at 0.33 ml/min in a 1.0 ml volume followed by a 0.5 ml sterile water flush. Total infusion time is 4.5 min.

Compositions for parenteral administration typically include 0.1% to 10% of actives and 90% to 99.9% of a carrier including a diluent and a solvent.

The amount of the carrier employed in conjunction with a disclosed compound is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

In vivo testing of candidate antisense or inhibitory nucleic acids may be conducted by means known to one of ordinary skill in the art. For example, the candidate one or more antisense or inhibitory nucleic acids may be administered to a mammal, such as a mouse or a rabbit. The mammal may be administered, by any route deemed appropriate, a dose of a candidate antisense or inhibitory nucleic acids. Conventional methods and criteria can then be used to monitor animals for signs of reduction or improvement of motor neuron activity and/or expression or activity of PIKFYVE gene or protein, respectively. If needed, the results obtained in the presence of the candidate antisense or inhibitory nucleic acids can be compared with results in control animals that are not treated with the candidate antisense or inhibitory nucleic acids. Dosing studies may be performed in, or in conjunction with, the herein described methods for identifying one or more antisense or inhibitory nucleic acids capable of treating a neurological disease and/or any follow-on testing of candidate antisense or inhibitory nucleic acids in vivo. One of skill in the art of medicine may determine the appropriate dosage of one or more antisense or inhibitory nucleic acids. The dosage may be determined by monitoring the subject for signs of disease inhibition or amelioration. The dosage may be increased or decreased to obtain the desired frequency of treatment. The toxicity and efficacy of one or more antisense or inhibitory nucleic acids may be determined by standard pharmaceutical procedures in cell cultures or experimental animals, e.g. determining the lethal dose to 50% of the population (LD50) and the dose therapeutically effective in 50% of the population (ED50). The dose ratio of LD50/ED50 is the therapeutic index and, indicating the ratio between the toxic and therapeutic effects. A delivery system may be designed to help prevent toxic side effects, by delivering the one or more antisense or inhibitory nucleic acids to specific targets, e.g., delivered specifically to motor or central nervous system neurons. The optimal dose of the one or more antisense or inhibitory nucleic acids may be determined based on results of clinical electrophysiology or electromyography to analyze excitability in peripheral nerves, for example.

The dosage for use in humans may be determined by evaluating data obtained from animal studies and cell culture assays. The preferred dosage will have little or no toxicity and include the ED50. The dosage may vary depending on the dosage form and route of administration. For any antisense or inhibitory nucleic acid used in the methods described herein, the dosage may be estimated initially in cell culture. A dose may be formulated in animal models that includes the concentration of the test compound which achieves a half maximal inhibition of symptoms (LD50) as determined in cell culture. Such information obtained from cell cultures and animal models may be used to more accurately determine useful doses in humans.

The present invention has multiple aspects, illustrated by the following non-limiting examples.

### EXAMPLES

Small molecule inhibitors of PIKFYVE kinase and antisense oligonucleotides (ASOs) that suppress PIKFYVE expression can prevent the degeneration of human and mouse neurons that carry a mutation in the C9ORF72 gene that leads to amyotrophic lateral sclerosis (ALS) and frontotemporal dementia (FTD).

ASOs are an attractive therapeutic option for neurodegenerative diseases because of their ease of delivery to the central nervous system and their relatively low exposure to the periphery. These properties maximize target engagement in the central nervous system and minimize undesired target engagement or off-target effects in the periphery.

The disclosure provides novel antisense oligonucleotide (ASO) sequences targeting the PIKFYVE gene that can suppress PIKFYVE expression in human cells. PIKFYVE ASOs can also rescue the survival of motor neurons derived from sporadic ALS patients. Moreover, PIKFYVE ASOs can lower levels of neurotoxic dipeptide repeat protein aggregates derived from the C9ORF72 repeat expansion in vivo in mice.

### Example 1

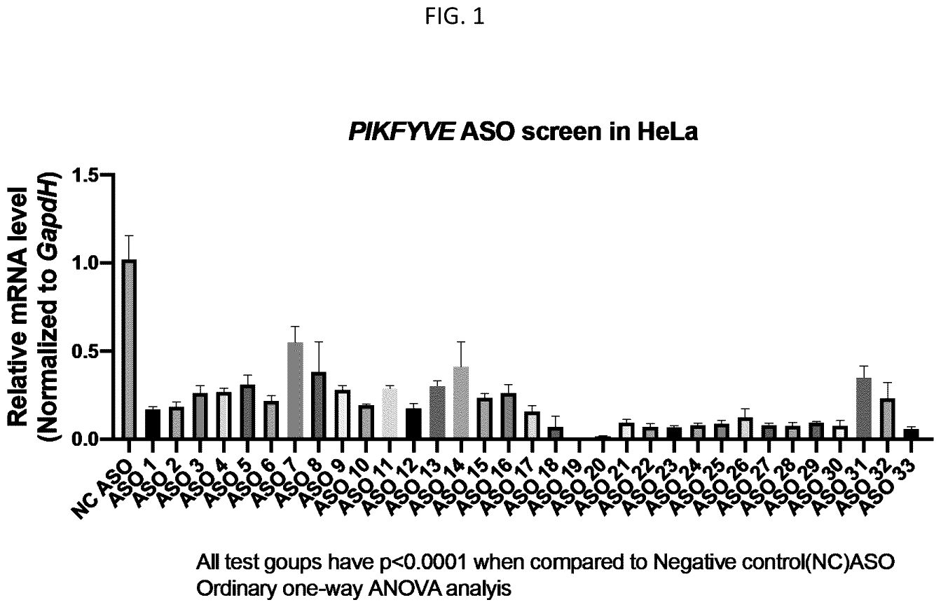

To identify ASO sequences that suppress PIKFYVE expression in human cells, ASOs were designed (see, Table 2) and synthesized as MOE gapmers, which contains sugar and linkage modifications that increase nuclease resistance and melting temperature while maintaining the ability to be used as a substrate of RNase H. The ability of each ASO to suppress PIKFYVE RNA levels was tested by transfecting them into human embryonic kidney 293T cells with Lipofectamine 2000 at a concentration of 100 nM and measuring PIKFYVE expression 7 days after transfection. As a control, NCASO was used. The relative PIKFYVE expression shown is an average of three technical replicates and values were calculated by normalizing to a GAPDH control. Together, these results, as shown in FIG. 1, show that several PIKFYVE ASOs (SEQ ID NO: 1-33, which correspond to ASO 1-33 in the figure) suppress PIKFYVE expression in human cells.

### Example 2

Suppression of off-target genes, such as CNTN5, was predicted in silico for various ASOs described herein. The results are provided in Table 3 below.

TABLE 3

| | (−) strand | (+) strand | Total | | |
|---|---|---|---|---|---|
| | comple- | comple- | comple- | | |
| | mentary | mentary | mentary | | |
| | binding | binding | binding | Total | Total |
| ASO | gene | gene | gene | intron | exon |
| Tofersen | 11 | 14 | 25 | 23 | 2 |
| Other PIKFYVE | 11 | 11 | 22 | 22 | 0 |
| ASO #1 | | | | | |
| Other PIKFYVE | 15 | 14 | 29 | 29 | 0 |
| ASO #2 | | | | | |
| ASO-2 | 0 | 3 | 3 | 3 | 0 |
| (SEQ ID NO: 2) | | | | | |
| ASO-4 | 3 | 4 | 7 | 6 | 1 |
| (SEQ ID NO: 4) | | | | | |
| ASO-13 | 0 | 2 | 2 | 2 | 0 |
| (SEQ ID NO: 13) | | | | | |
| ASO-20 | 2 | 3 | 5 | 5 | 0 |
| (SEQ ID NO: 20) | | | | | |
| ASO-21 | 1 | 4 | 5 | 4 | 1 |
| (SEQ ID NO: 21) | | | | | |
| ASO-26 | 0 | 3 | 3 | 3 | 0 |
| (SEQ ID NO: 26) | | | | | |
| ASO-27 (SEQ ID | 1 | 2 | 3 | 2 | 1 |
| NO: 27) | | | | | |
| ASO-29 | 1 | 11 | 12 | 12 | 0 |
| (SEQ ID NO: 29) | | | | | |
| ASO-105 | 1 | 5 | 6 | 6 | 0 |
| (SEQ ID NO: 105) | | | | | |

Five (5) off-target gene candidates were predicted for ASO-520 (SEQ ID NO:520) from sequence analysis (as compared to 25 for tofersen). Two of these genes have very low expression in the brain and are not detectable in induced neurons. To assess actual off-target suppression for the remaining three genes (ZNF385D, ERC2, and AKAP6), the effect of ASO-520 treatment on the expression of these three genes and PIKFYVE was tested in a patient induced neuron line by qPCR. ASO-520 did not significantly affect their expression at doses that reduced PIKFYVE by 50%.

### Example 3

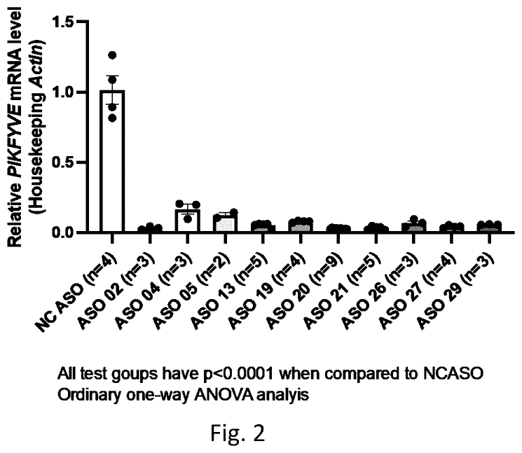

In the study, neonate transgenic hPIKFYVE BAC mice received 25 μg of negative control ASO or test compound by intracerebroventricular (ICV) injection at P1 (postnatal day 1) and tissue samples were collected 14 days post-treatment. As illustrated in FIG. 2, the ASOs tested were potent PIKFYVE suppressors. At dosages from 0.0004 μg to 25 μg of ASO-520, a dose-dependent PIKFYVE mRNA level reduction was observed in the mice.

### Example 4

A TDP-43 mouse model that develops neurodegeneration, motor impairment, and paralysis was used to assess efficacy of PIKFYVE suppresion. Wils et al., “TDP-43 Transgenic Mice Develop Spastic Paralysis and Neuronal Inclusions Characteristic of ALS and Frontotemporal Lobar Degeneration.” PNAS 107(8):3858-63, 2010.

The mice were further genetically modified to delete one coy of PIKFYVE. This deletion significantly rescued motor function in TDP-43 mice, extended mean survival by 28% and reduced the risk of death (hR: hazard ratio) by 73%. The deletion did not cause any motor, cognitive, or health problems in wild-type (WT) mice.

Intracerebroventricular injection of 25 μg of a mPIKFYVE-targeting ASO at postnatal day 1 (5 μg/μl concentration in the central nervous system) significantly decreased PIKFYVE expression by ˜50% compared to a negative control (NC) ASO. This PIKFYVE ASO treatment significantly rescued motor function and survival in TDP-43 mice at levels similar to that of the genetic deletion, and did not alter function in WT mice. A 5-fold lower dose of 5 μg of PIKFYVE ASO also significantly rescued motor function and survival at levels similar to the genetic deletion. This established that the ASO has at least a 5-fold therapeutic window in this model.

Histological analysis showed that the number of pathological pTDP-43 aggregates, elevated in the TDP-43 mouse, are significantly lowered with mPIKFYVE ASO treatment and completely rescued to the level of WT mice. Total TDP-43 localization, pathologically localized to the cytoplasm in the TDP-43 mouse, is significantly re-localized to the nucleus upon treatment. The number of motor neurons in the lateral motor column of the spinal cord ventral horn area was fully rescued to the level of WT in the PIKFYVE ASO treated mice.

### Example 5

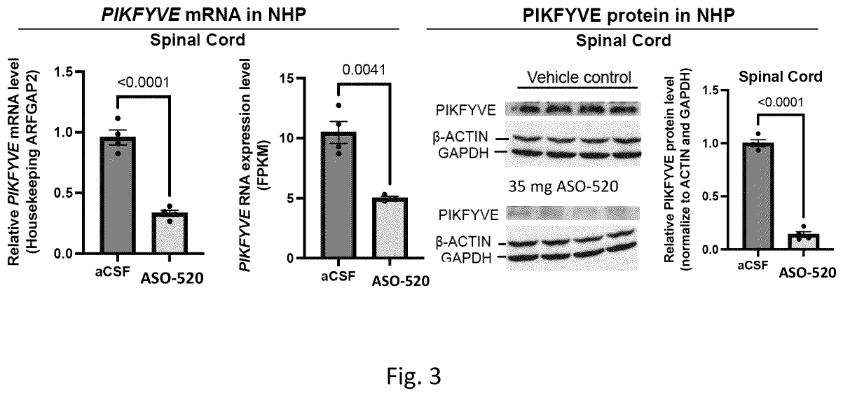

35 mg of ASO-520 was administered intrathecally to non-human primates every other week for two weeks (2 doses). This resulted in reduction in PIKFYVE mRNA and 80% suppression of PIKFYVE, as shown by FIG. 3, with no observed adverse events (including in brain and spinal cord histopathology). (One outlier was removed from the RNA-seq data.)

### Example 6

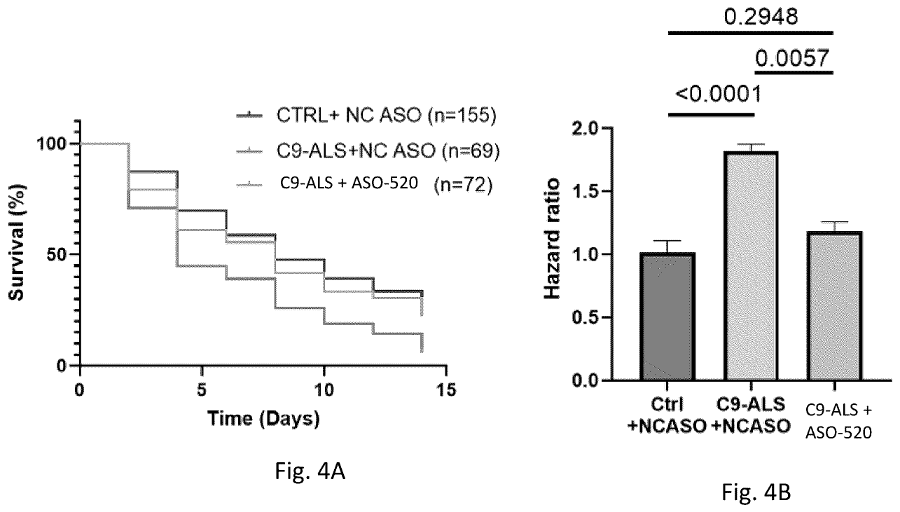

The ability of motor neurons to survive in the presence of a non-coding ASO or AS-520 (SEQ ID NO: 520) was determined. FIG. 4A shows the percent survival of control motor neurons in the presence of a non-coding ASO (NC ASO) or C9ALS patient-derived motor neurons in the presence of (i) a NC ASO or (ii) AS-520. A greater number of C9ALS patient-derived motor neurons survived in the presence of ASO-520 than in the presence of NC ASO. FIG. 4B shows the hazard ratio for control motor neurons in the presence of a NC ASO or C9ALS patient-derived motor neurons in the presence of (i) a NC ASO or (ii) AS-520 (SEQ ID NO: 520). The hazard ratio of C9ALS patient-derived motor neurons in the presence of AS-20 was significantly lower than in the presence of NC ASO.

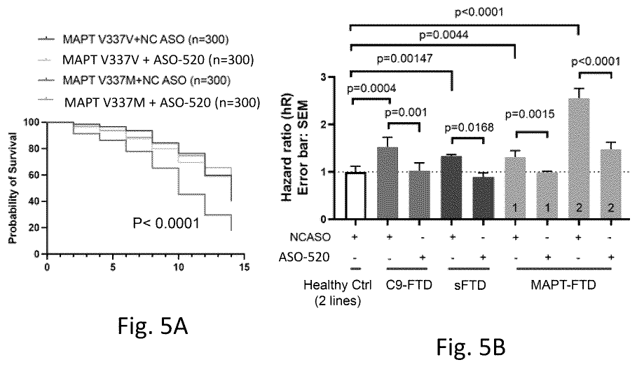

The ability of FTD patient-derived cortical neurons to survive in the presence of a non-coding ASO or AS-520 (SEQ ID NO: 520) was determined. FIG. 5A is a graph showing the probability of survival for FTD patient-derived cortical neurons with MAPT V337V or V337M in the present of NC ASO or AS-520 (SEQ ID NO: 520). AS-520 increased the probability of survival in MAPT V337M cortical neurons compared to the NC ASO. FIG. 5B is a bar graph showing the hazard ratio for cortical neurons from control, C9orf72-associated FTD (C9-FTD), sporadic FTD (sFTD), and microtubule-associated protein tau (MAPT)-associated FTD (MAPT-FTD) patients treated with NC ASO or AS-520. AS-520 significantly decreased the hazard ratio in the C9-FTD, sFTD and MAPT-FTD cortical neurons.

The disclosure provides ASOs that suppress PIKFYVE expression in human cells. The accompany data suggest that these ASOs may be capable of preventing neurodegeneration in ALS and FTD patients.

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention. All references cited herein are incorporated by reference.

1. A single stranded antisense oligonucleotide of 20 nucleotides in length that suppresses the expression of a PIKFYVE, wherein the antisense oligonucleotide comprises a nucleobase sequence that comprises the sequence set forth in any of the nucleobase sequences of SEQ ID NOs: 1-500.

2-22. (canceled)

23. A pharmaceutical composition comprising the antisense oligonucleotide of claim 1, and a pharmaceutically acceptable carrier, diluent and/or excipient.

24-25. (canceled)

26. A method of treating a subject having a neurological or neurodegenerative disease in need thereof comprising administering a therapeutically effective amount of the antisense oligonucleotide of claim 1.

27. The method of claim 26, wherein the neurological disease is associated with neuronal hyperexcitability.

28. The method of claim 26, wherein the neurological disease is associated with aberrant endosomal trafficking.

29. The method of claim 26, wherein the neurological disease is associated with aberrant lysosomal trafficking.

30. The method of claim 26, wherein the neurological disease comprises familial amyotrophic lateral sclerosis (ALS), sporadic amyotrophic lateral sclerosis, familial frontotemporal dementia (FTD), sporadic frontotemporal dementia, progressive supranuclear palsy, Alzheimer's disease, chronic traumatic encephalopathy, Parkinson's disease, Charcot Marie Tooth 2A and 4B, Huntington's disease, dementia, transmissible spongiform encephalopathy, spinobulbar muscular atrophy, dentatorubral-pallidoluysian atrophy, spinocerebellar ataxias, or Creutzfeldt-Jakob disease.

31. The method of claim 26, wherein the neurological disease is familial amyotrophic lateral sclerosis.

32. The method of claim 26, wherein the neurological disease is sporadic amyotrophic lateral sclerosis.

33. The method of claim 26, wherein the neurological disease is familial frontotemporal dementia.

34. The method of claim 26, wherein the neurological disease is sporadic frontotemporal dementia.

35. The method of claim 26, wherein the neurological disease is frontotemporal dementia with TDP-43 pathology.

36. The method of claim 26, wherein the neurological disease is frontotemporal dementia with tau pathology.

37. The method of claim 26, wherein the subject is haploinsufficient for the C9ORF72 gene.

38. The method of claim 26, wherein the subject has a GGGGCC repeat expansion in C90RF72.

39. The method of claim 26, wherein the subject has a (GGGGCC)n (SEQ ID NO: 534) hexanucleotide expansion in C90RF72, where n is at least 30.

40. The method of claim 26, wherein the subject has C9orf72-associated frontotemporal dementia.

41. The method of claim 26, wherein the subject has microtubule associated protein tau (MAPT)-associated frontotemporal dementia.

42. The method of claim 41, wherein the patient has the V337M MAPT mutation.

43. A method of inhibiting or suppressing expression of PIKFYVE in a patient having a neurological or neurodegenerative disease comprising administering an effective amount of the antisense oligonucleotide of claim 1.

44. (canceled)


SEQUENCE LISTING (534 sequences)

SEQ ID NO: 1 - DNA, 20 bp, synthetic construct
  misc_feature 1..20: note = Description of Artificial Sequence: Synthetic oligonucleotide
  source 1..20: mol_type = other DNA
atggcatgatccccataagc

SEQ ID NO: 2 - DNA, 20 bp, synthetic construct
  misc_feature 1..20: note = Description of Artificial Sequence: Synthetic oligonucleotide
  source 1..20: mol_type = other DNA
aggaccagtacaacctgtag

SEQ ID NO: 3 - DNA, 20 bp, synthetic construct
  misc_feature 1..20: note = Description of Artificial Sequence: Synthetic oligonucleotide
  source 1..20: mol_type = other DNA
aggtgccccaacttgttacc

SEQ ID NO: 4 - DNA, 20 bp, synthetic construct
  misc_feature 1..20: note = Description of Artificial Sequence: Synthetic oligonucleotide
  source 1..20: mol_type = other DNA
actaaggtgccccaacttgt

SEQ ID NO: 5 - DNA, 20 bp, synthetic construct
  misc_feature 1..20: note = Description of Artificial Sequence: Synthetic oligonucleotide
  source 1..20: mol_type = other DNA
accaggtagtcccaaataac

SEQ ID NO: 6 - DNA, 20 bp, synthetic construct
  misc_feature 1..20: note = Description of Artificial Sequence: Synthetic oligonucleotide
  source 1..20: mol_type = other DNA
gcctgacccctatacttgac

SEQ ID NO: 7 - DNA, 20 bp, synthetic construct
  misc_feature 1..20: note = Description of Artificial Sequence: Synthetic oligonucleotide
  source 1..20: mol_type = other DNA
ctgacccctatacttgacag

SEQ ID NO: 8 - DNA, 20 bp, synthetic construct
  misc_feature 1..20: note = Description of Artificial Sequence: Synthetic oligonucleotide
  source 1..20: mol_type = other DNA
ttaagccctttggtccaacc

SEQ ID NO: 9 - DNA, 20 bp, synthetic construct
  misc_feature 1..20: note = Description of Artificial Sequence: Synthetic oligonucleotide
  source 1..20: mol_type = other DNA
gagttagcaccctctactac

SEQ ID NO: 10 - DNA, 20 bp, synthetic construct
  misc_feature 1..20: note = Description of Artificial Sequence: Synthetic oligonucleotide
  source 1..20: mol_type = other DNA
gtcataagtccttggtcaac

SEQ ID NO: 11 - DNA, 20 bp, synthetic construct
  misc_feature 1..20: note = Description of Artificial Sequence: Synthetic oligonucleotide
  source 1..20: mol_type = other DNA
aagcctgacccctatacttg

SEQ ID NO: 12 - DNA, 20 bp, synthetic construct
  misc_feature 1..20: note = Description of Artificial Sequence: Synthetic oligonucleotide
  source 1..20: mol_type = other DNA
agcctgacccctatacttga

SEQ ID NO: 13 - DNA, 20 bp, synthetic construct
  misc_feature 1..20: note = Description of Artificial Sequence: Synthetic oligonucleotide
  source 1..20: mol_type = other DNA
catcctattagcttaaaccc

SEQ ID NO: 14 - DNA, 20 bp, synthetic construct
  misc_feature 1..20: note = Description of Artificial Sequence: Synthetic oligonucleotide
  source 1..20: mol_type = other DNA
cctgacccctatacttgaca

SEQ ID NO: 15 - DNA, 20 bp, synthetic construct
  misc_feature 1..20: note = Description of Artificial Sequence: Synthetic oligonucleotide
  source 1..20: mol_type = other DNA
tatgatctgatagccaactc

SEQ ID NO: 16 - DNA, 20 bp, synthetic construct
  misc_feature 1..20: note = Description of Artificial Sequence: Synthetic oligonucleotide
  source 1..20: mol_type = other DNA
gtaccaggaccagtacaacc

SEQ ID NO: 17 - DNA, 20 bp, synthetic construct
  misc_feature 1..20: note = Description of Artificial Sequence: Synthetic oligonucleotide
  source 1..20: mol_type = other DNA
caggaccagtacaacctgta

SEQ ID NO: 18 - DNA, 20 bp, synthetic construct
  misc_feature 1..20: note = Description of Artificial Sequence: Synthetic oligonucleotide
  source 1..20: mol_type = other DNA
acaccctttggagtgtctag

SEQ ID NO: 19 - DNA, 20 bp, synthetic construct
  misc_feature 1..20: note = Description of Artificial Sequence: Synthetic oligonucleotide
  source 1..20: mol_type = other DNA
gccccaacttgttacctaag

SEQ ID NO: 20 - DNA, 20 bp, synthetic construct
  misc_feature 1..20: note = Description of Artificial Sequence: Synthetic oligonucleotide
  source 1..20: mol_type = other DNA
ccccaatctagttcaagcat

SEQ ID NO: 21 - DNA, 20 bp, synthetic construct
  misc_feature 1..20: note = Description of Artificial Sequence: Synthetic oligonucleotide
  source 1..20: mol_type = other DNA
tccaagagttagcaccctct

SEQ ID NO: 22 - DNA, 20 bp, synthetic construct
  misc_feature 1..20: note = Description of Artificial Sequence: Synthetic oligonucleotide
  source 1..20: mol_type = other DNA
ggaccagtacaacctgtagt

SEQ ID NO: 23 - DNA, 20 bp, synthetic construct
  misc_feature 1..20: note = Description of Artificial Sequence: Synthetic oligonucleotide
  source 1..20: mol_type = other DNA
aaacccagtgtagctccatt

SEQ ID NO: 24 - DNA, 20 bp, synthetic construct
  misc_feature 1..20: note = Description of Artificial Sequence: Synthetic oligonucleotide
  source 1..20: mol_type = other DNA
tgccccaacttgttacctaa

SEQ ID NO: 25 - DNA, 20 bp, synthetic construct
  misc_feature 1..20: note = Description of Artificial Sequence: Synthetic oligonucleotide
  source 1..20: mol_type = other DNA
ccaatagcaagcagcctata

SEQ ID NO: 26 - DNA, 20 bp, synthetic construct
  misc_feature 1..20: note = Description of Artificial Sequence: Synthetic oligonucleotide
  source 1..20: mol_type = other DNA
ctacaatcaaatcctggtag

SEQ ID NO: 27 - DNA, 20 bp, synthetic construct
  misc_feature 1..20: note = Description of Artificial Sequence: Synthetic oligonucleotide
  source 1..20: mol_type = other DNA
gaccaagtttatggacccca

SEQ ID NO: 28 - DNA, 20 bp, synthetic construct
  misc_feature 1..20: note = Description of Artificial Sequence: Synthetic oligonucleotide
  source 1..20: mol_type = other DNA
gtgaccaccatctatagtta

SEQ ID NO: 29 - DNA, 20 bp, synthetic construct
  misc_feature 1..20: note = Description of Artificial Sequence: Synthetic oligonucleotide
  source 1..20: mol_type = other DNA
ctaaggtaactgttcctaga

SEQ ID NO: 30 - DNA, 20 bp, synthetic construct
  misc_feature 1..20: note = Description of Artificial Sequence: Synthetic oligonucleotide
  source 1..20: mol_type = other DNA
tgaccaagtttatggacccc

SEQ ID NO: 31 - DNA, 20 bp, synthetic construct
  misc_feature 1..20: note = Description of Artificial Sequence: Synthetic oligonucleotide
  source 1..20: mol_type = other DNA
accaagtttatggaccccaa

SEQ ID NO: 32 - DNA, 20 bp, synthetic construct
  misc_feature 1..20: note = Description of Artificial Sequence: Synthetic oligonucleotide
  source 1..20: mol_type = other DNA
gcaataaagctaaccacatc

SEQ ID NO: 33 - DNA, 20 bp, synthetic construct
  misc_feature 1..20: note = Description of Artificial Sequence: Synthetic oligonucleotide
  source 1..20: mol_type = other DNA
gattctaccacacagtacac

SEQ ID NO: 34 - DNA, 20 bp, synthetic construct
  misc_feature 1..20: note = Description of Artificial Sequence: Synthetic oligonucleotide
  source 1..20: mol_type = other DNA
gagccctaactgtatcttcc

SEQ ID NO: 35 - DNA, 20 bp, synthetic construct
  misc_feature 1..20: note = Description of Artificial Sequence: Synthetic oligonucleotide
  source 1..20: mol_type = other DNA
tagctgattgccccttaaca

SEQ ID NO: 36 - DNA, 20 bp, synthetic construct
  misc_feature 1..20: note = Description of Artificial Sequence: Synthetic oligonucleotide
  source 1..20: mol_type = other DNA
gagactgctaaggctactac

SEQ ID NO: 37 - DNA, 20 bp, synthetic construct
  misc_feature 1..20: note = Description of Artificial Sequence: Synthetic oligonucleotide
  source 1..20: mol_type = other DNA
gctaagccctaagtatatcc

SEQ ID NO: 38 - DNA, 20 bp, synthetic construct
  misc_feature 1..20: note = Description of Artificial Sequence: Synthetic oligonucleotide
  source 1..20: mol_type = other DNA
actactggtaaggcagtccc

SEQ ID NO: 39 - DNA, 20 bp, synthetic construct
  misc_feature 1..20: note = Description of Artificial Sequence: Synthetic oligonucleotide
  source 1..20: mol_type = other DNA
ggtatccctacacttctaca

SEQ ID NO: 40 - DNA, 20 bp, synthetic construct
  misc_feature 1..20: note = Description of Artificial Sequence: Synthetic oligonucleotide
  source 1..20: mol_type = other DNA
gagtaccaggaccagtacaa

SEQ ID NO: 41 - DNA, 20 bp, synthetic construct
  misc_feature 1..20: note = Description of Artificial Sequence: Synthetic oligonucleotide
  source 1..20: mol_type = other DNA
ataacccccctgctaagagc

SEQ ID NO: 42 - DNA, 20 bp, synthetic construct
  misc_feature 1..20: note = Description of Artificial Sequence: Synthetic oligonucleotide
  source 1..20: mol_type = other DNA
cttagctgattgccccttaa

SEQ ID NO: 43 - DNA, 20 bp, synthetic construct
  misc_feature 1..20: note = Description of Artificial Sequence: Synthetic oligonucleotide
  source 1..20: mol_type = other DNA
gctatactactagaagaacc

SEQ ID NO: 44 - DNA, 20 bp, synthetic construct
  misc_feature 1..20: note = Description of Artificial Sequence: Synthetic oligonucleotide
  source 1..20: mol_type = other DNA
gtacctaaatacaggtccta

SEQ ID NO: 45 - DNA, 20 bp, synthetic construct
  misc_feature 1..20: note = Description of Artificial Sequence: Synthetic oligonucleotide
  source 1..20: mol_type = other DNA
aggtatccctacacttctac

SEQ ID NO: 46 - DNA, 20 bp, synthetic construct
  misc_feature 1..20: note = Description of Artificial Sequence: Synthetic oligonucleotide
  source 1..20: mol_type = other DNA
gcctacatccagttgattag

SEQ ID NO: 47 - DNA, 20 bp, synthetic construct
  misc_feature 1..20: note = Description of Artificial Sequence: Synthetic oligonucleotide
  source 1..20: mol_type = other DNA
gagtggtaatcagtcctatt

SEQ ID NO: 48 - DNA, 20 bp, synthetic construct
  misc_feature 1..20: note = Description of Artificial Sequence: Synthetic oligonucleotide
  source 1..20: mol_type = other DNA
gacaaagtcctacctggtac

SEQ ID NO: 49 - DNA, 20 bp, synthetic construct
  misc_feature 1..20: note = Description of Artificial Sequence: Synthetic oligonucleotide
  source 1..20: mol_type = other DNA
gtaatctgttgagatacacc

SEQ ID NO: 50 - DNA, 20 bp, synthetic construct
  misc_feature 1..20: note = Description of Artificial Sequence: Synthetic oligonucleotide
  source 1..20: mol_type = other DNA
tccttaataccccaggttat

SEQ ID NO: 51 - DNA, 20 bp, synthetic construct
  misc_feature 1..20: note = Description of Artificial Sequence: Synthetic oligonucleotide
  source 1..20: mol_type = other DNA
ttccataactaaggtgcccc

SEQ ID NO: 52 - DNA, 20 bp, synthetic construct
  misc_feature 1..20: note = Description of Artificial Sequence: Synthetic oligonucleotide
  source 1..20: mol_type = other DNA
aactaaggtgccccaacttg

SEQ ID NO: 53 - DNA, 20 bp, synthetic construct
  misc_feature 1..20: note = Description of Artificial Sequence: Synthetic oligonucleotide
  source 1..20: mol_type = other DNA
gtgccccaacttgttaccta

SEQ ID NO: 54 - DNA, 20 bp, synthetic construct
  misc_feature 1..20: note = Description of Artificial Sequence: Synthetic oligonucleotide
  source 1..20: mol_type = other DNA
caagtccctataacataacc

SEQ ID NO: 55 - DNA, 20 bp, synthetic construct
  misc_feature 1..20: note = Description of Artificial Sequence: Synthetic oligonucleotide
  source 1..20: mol_type = other DNA
gagcagtcagatgtagttcc

SEQ ID NO: 56 - DNA, 20 bp, synthetic construct
  misc_feature 1..20: note = Description of Artificial Sequence: Synthetic oligonucleotide
  source 1..20: mol_type = other DNA
aaggatggtgtaggttccaa

SEQ ID NO: 57 - DNA, 20 bp, synthetic construct
  misc_feature 1..20: note = Description of Artificial Sequence: Synthetic oligonucleotide
  source 1..20: mol_type = other DNA
taacccactagaatagcacc

SEQ ID NO: 58 - DNA, 20 bp, synthetic construct
  misc_feature 1..20: note = Description of Artificial Sequence: Synthetic oligonucleotide
  source 1..20: mol_type = other DNA
ggtatgcccaccaaagttgt

SEQ ID NO: 59 - DNA, 20 bp, synthetic construct
  misc_feature 1..20: note = Description of Artificial Sequence: Synthetic oligonucleotide
  source 1..20: mol_type = other DNA
gtacaccttagtctaacagt

SEQ ID NO: 60 - DNA, 20 bp, synthetic construct
  misc_feature 1..20: note = Description of Artificial Sequence: Synthetic oligonucleotide
  source 1..20: mol_type = other DNA
accaaggtactctgaaccta

SEQ ID NO: 61 - DNA, 20 bp, synthetic construct
  misc_feature 1..20: note = Description of Artificial Sequence: Synthetic oligonucleotide
  source 1..20: mol_type = other DNA
tgagtggtaatcagtcctat

SEQ ID NO: 62 - DNA, 20 bp, synthetic construct
  misc_feature 1..20: note = Description of Artificial Sequence: Synthetic oligonucleotide
  source 1..20: mol_type = other DNA
tacccttacctaatcaagag

SEQ ID NO: 63 - DNA, 20 bp, synthetic construct
  misc_feature 1..20: note = Description of Artificial Sequence: Synthetic oligonucleotide
  source 1..20: mol_type = other DNA
gtgagccctaactgtatctt

SEQ ID NO: 64 - DNA, 20 bp, synthetic construct
  misc_feature 1..20: note = Description of Artificial Sequence: Synthetic oligonucleotide
  source 1..20: mol_type = other DNA
aataacccccctgctaagag

SEQ ID NO: 65 - DNA, 20 bp, synthetic construct
  misc_feature 1..20: note = Description of Artificial Sequence: Synthetic oligonucleotide
  source 1..20: mol_type = other DNA
aaccaatagtgacttatggc

SEQ ID NO: 66 - DNA, 20 bp, synthetic construct
  misc_feature 1..20: note = Description of Artificial Sequence: Synthetic oligonucleotide
  source 1..20: mol_type = other DNA
ttagctgattgccccttaac

SEQ ID NO: 67 - DNA, 20 bp, synthetic construct
  misc_feature 1..20: note = Description of Artificial Sequence: Synthetic oligonucleotide
  source 1..20: mol_type = other DNA
agctgattgccccttaacag

SEQ ID NO: 68 - DNA, 20 bp, synthetic construct
  misc_feature 1..20: note = Description of Artificial Sequence: Synthetic oligonucleotide
  source 1..20: mol_type = other DNA
gctagtttacatacctgtcc

SEQ ID NO: 69 - DNA, 20 bp, synthetic construct
  misc_feature 1..20: note = Description of Artificial Sequence: Synthetic oligonucleotide
  source 1..20: mol_type = other DNA
ccttaataccccaggttatc

SEQ ID NO: 70 - DNA, 20 bp, synthetic construct
  misc_feature 1..20: note = Description of Artificial Sequence: Synthetic oligonucleotide
  source 1..20: mol_type = other DNA
taactaaggtgccccaactt

SEQ ID NO: 71 - DNA, 20 bp, synthetic construct
  misc_feature 1..20: note = Description of Artificial Sequence: Synthetic oligonucleotide
  source 1..20: mol_type = other DNA
taaggtgccccaacttgtta

SEQ ID NO: 72 - DNA, 20 bp, synthetic construct
  misc_feature 1..20: note = Description of Artificial Sequence: Synthetic oligonucleotide
  source 1..20: mol_type = other DNA
cccaacttgttacctaagca

SEQ ID NO: 73 - DNA, 20 bp, synthetic construct
  misc_feature 1..20: note = Description of Artificial Sequence: Synthetic oligonucleotide
  source 1..20: mol_type = other DNA
gttggcacatcacactatta

SEQ ID NO: 74 - DNA, 20 bp, synthetic construct
  misc_feature 1..20: note = Description of Artificial Sequence: Synthetic oligonucleotide
  source 1..20: mol_type = other DNA
atgttgaggctgtcacacta

SEQ ID NO: 75 - DNA, 20 bp, synthetic construct
  misc_feature 1..20: note = Description of Artificial Sequence: Synthetic oligonucleotide
  source 1..20: mol_type = other DNA
ctaacctaaggtaactgttc

SEQ ID NO: 76 - DNA, 20 bp, synthetic construct
  misc_feature 1..20: note = Description of Artificial Sequence: Synthetic oligonucleotide
  source 1..20: mol_type = other DNA
aggactagagaccacttaac

SEQ ID NO: 77 - DNA, 20 bp, synthetic construct
  misc_feature 1..20: note = Description of Artificial Sequence: Synthetic oligonucleotide
  source 1..20: mol_type = other DNA
acttaacctccccctacatc

SEQ ID NO: 78 - DNA, 20 bp, synthetic construct
  misc_feature 1..20: note = Description of Artificial Sequence: Synthetic oligonucleotide
  source 1..20: mol_type = other DNA
atggtgtaggttccaaaccc

SEQ ID NO: 79 - DNA, 20 bp, synthetic construct
  misc_feature 1..20: note = Description of Artificial Sequence: Synthetic oligonucleotide
  source 1..20: mol_type = other DNA
ggatctactcctactactcc

SEQ ID NO: 80 - DNA, 20 bp, synthetic construct
  misc_feature 1..20: note = Description of Artificial Sequence: Synthetic oligonucleotide
  source 1..20: mol_type = other DNA
tgacccctatacttgacaga

SEQ ID NO: 81 - DNA, 20 bp, synthetic construct
  misc_feature 1..20: note = Description of Artificial Sequence: Synthetic oligonucleotide
  source 1..20: mol_type = other DNA
cctttggtccaacctataat

SEQ ID NO: 82 - DNA, 20 bp, synthetic construct
  misc_feature 1..20: note = Description of Artificial Sequence: Synthetic oligonucleotide
  source 1..20: mol_type = other DNA
ggtaaaacctgacctatgtc

SEQ ID NO: 83 - DNA, 20 bp, synthetic construct
  misc_feature 1..20: note = Description of Artificial Sequence: Synthetic oligonucleotide
  source 1..20: mol_type = other DNA
agcccaataatggagttgac

SEQ ID NO: 84 - DNA, 20 bp, synthetic construct
  misc_feature 1..20: note = Description of Artificial Sequence: Synthetic oligonucleotide
  source 1..20: mol_type = other DNA
gcccaataatggagttgaca

SEQ ID NO: 85 - DNA, 20 bp, synthetic construct
  misc_feature 1..20: note = Description of Artificial Sequence: Synthetic oligonucleotide
  source 1..20: mol_type = other DNA
ttactggacagatcccttac

SEQ ID NO: 86 - DNA, 20 bp, synthetic construct
  misc_feature 1..20: note = Description of Artificial Sequence: Synthetic oligonucleotide
  source 1..20: mol_type = other DNA
ctgagtggtaatcagtccta

SEQ ID NO: 87 - DNA, 20 bp, synthetic construct
  misc_feature 1..20: note = Description of Artificial Sequence: Synthetic oligonucleotide
  source 1..20: mol_type = other DNA
agagttagcaccctctacta

SEQ ID NO: 88 - DNA, 20 bp, synthetic construct
  misc_feature 1..20: note = Description of Artificial Sequence: Synthetic oligonucleotide
  source 1..20: mol_type = other DNA
ccaatgctggtcagttgctc

SEQ ID NO: 89 - DNA, 20 bp, synthetic construct
  misc_feature 1..20: note = Description of Artificial Sequence: Synthetic oligonucleotide
  source 1..20: mol_type = other DNA
acatagtccaatacccttac

SEQ ID NO: 90 - DNA, 20 bp, synthetic construct
  misc_feature 1..20: note = Description of Artificial Sequence: Synthetic oligonucleotide
  source 1..20: mol_type = other DNA
actgccattaggactgttac

SEQ ID NO: 91 - DNA, 20 bp, synthetic construct
  misc_feature 1..20: note = Description of Artificial Sequence: Synthetic oligonucleotide
  source 1..20: mol_type = other DNA
gaaagtagttggactctccc

SEQ ID NO: 92 - DNA, 20 bp, synthetic construct
  misc_feature 1..20: note = Description of Artificial Sequence: Synthetic oligonucleotide
  source 1..20: mol_type = other DNA
tagttggactctcccagtgc

SEQ ID NO: 93 - DNA, 20 bp, synthetic construct
  misc_feature 1..20: note = Description of Artificial Sequence: Synthetic oligonucleotide
  source 1..20: mol_type = other DNA
gctccaagtactatgtcaac

SEQ ID NO: 94 - DNA, 20 bp, synthetic construct
  misc_feature 1..20: note = Description of Artificial Sequence: Synthetic oligonucleotide
  source 1..20: mol_type = other DNA
ggatagaggattcaaggctc

SEQ ID NO: 95 - DNA, 20 bp, synthetic construct
  misc_feature 1..20: note = Description of Artificial Sequence: Synthetic oligonucleotide
  source 1..20: mol_type = other DNA
taacccccctgctaagagcc

SEQ ID NO: 96 - DNA, 20 bp, synthetic construct
  misc_feature 1..20: note = Description of Artificial Sequence: Synthetic oligonucleotide
  source 1..20: mol_type = other DNA
actcaagaaccaacctgtag

SEQ ID NO: 97 - DNA, 20 bp, synthetic construct
  misc_feature 1..20: note = Description of Artificial Sequence: Synthetic oligonucleotide
  source 1..20: mol_type = other DNA
tgctacaacccaactccccc

SEQ ID NO: 98 - DNA, 20 bp, synthetic construct
  misc_feature 1..20: note = Description of Artificial Sequence: Synthetic oligonucleotide
  source 1..20: mol_type = other DNA
gattgccccttaacagaacc

SEQ ID NO: 99 - DNA, 20 bp, synthetic construct
  misc_feature 1..20: note = Description of Artificial Sequence: Synthetic oligonucleotide
  source 1..20: mol_type = other DNA
gtttagacttgccacactaa

SEQ ID NO: 100 - DNA, 20 bp, synthetic construct
  misc_feature 1..20: note = Description of Artificial Sequence: Synthetic oligonucleotide
  source 1..20: mol_type = other DNA
taagccctgtcagcacaagg

SEQ ID NO: 101 - DNA, 20 bp, synthetic construct
  misc_feature 1..20: note = Description of Artificial Sequence: Synthetic oligonucleotide
  source 1..20: mol_type = other DNA
ggtcttgtagacaccaataa

SEQ ID NO: 102 - DNA, 20 bp, synthetic construct
  misc_feature 1..20: note = Description of Artificial Sequence: Synthetic oligonucleotide
  source 1..20: mol_type = other DNA
cttaataccccaggttatct

SEQ ID NO: 103 - DNA, 20 bp, synthetic construct
  misc_feature 1..20: note = Description of Artificial Sequence: Synthetic oligonucleotide
  source 1..20: mol_type = other DNA
ttaataccccaggttatctc

SEQ ID NO: 104 - DNA, 20 bp, synthetic construct
  misc_feature 1..20: note = Description of Artificial Sequence: Synthetic oligonucleotide
  source 1..20: mol_type = other DNA
cataactaaggtgccccaac

SEQ ID NO: 105 - DNA, 20 bp, synthetic construct
  misc_feature 1..20: note = Description of Artificial Sequence: Synthetic oligonucleotide
  source 1..20: mol_type = other DNA
agtggagtactatggactaa

SEQ ID NO: 106 - DNA, 20 bp, synthetic construct
  misc_feature 1..20: note = Description of Artificial Sequence: Synthetic oligonucleotide
  source 1..20: mol_type = other DNA
gcccatactcaagtttatcc

SEQ ID NO: 107 - DNA, 20 bp, synthetic construct
  misc_feature 1..20: note = Description of Artificial Sequence: Synthetic oligonucleotide
  source 1..20: mol_type = other DNA
caatgttgaggctgtcacac

SEQ ID NO: 108 - DNA, 20 bp, synthetic construct
  misc_feature 1..20: note = Description of Artificial Sequence: Synthetic oligonucleotide
  source 1..20: mol_type = other DNA
ggcatagcttatggatcaaa

SEQ ID NO: 109 - DNA, 20 bp, synthetic construct
  misc_feature 1..20: note = Description of Artificial Sequence: Synthetic oligonucleotide
  source 1..20: mol_type = other DNA
ggctttcactccaccagatt

SEQ ID NO: 110 - DNA, 20 bp, synthetic construct
  misc_feature 1..20: note = Description of Artificial Sequence: Synthetic oligonucleotide
  source 1..20: mol_type = other DNA
gacccatctctcaggtgatc

SEQ ID NO: 111 - DNA, 20 bp, synthetic construct
  misc_feature 1..20: note = Description of Artificial Sequence: Synthetic oligonucleotide
  source 1..20: mol_type = other DNA
tagcacaggtatccctacac

SEQ ID NO: 112 - DNA, 20 bp, synthetic construct
  misc_feature 1..20: note = Description of Artificial Sequence: Synthetic oligonucleotide
  source 1..20: mol_type = other DNA
gcctgtagccctcccctaaa

SEQ ID NO: 113 - DNA, 20 bp, synthetic construct
  misc_feature 1..20: note = Description of Artificial Sequence: Synthetic oligonucleotide
  source 1..20: mol_type = other DNA
tcccagttggtaattccacc

SEQ ID NO: 114 - DNA, 20 bp, synthetic construct
  misc_feature 1..20: note = Description of Artificial Sequence: Synthetic oligonucleotide
  source 1..20: mol_type = other DNA
cccagttggtaattccaccc

SEQ ID NO: 115 - DNA, 20 bp, synthetic construct
  misc_feature 1..20: note = Description of Artificial Sequence: Synthetic oligonucleotide
  source 1..20: mol_type = other DNA
tggtaattccacccctccaa

SEQ ID NO: 116 - DNA, 20 bp, synthetic construct
  misc_feature 1..20: note = Description of Artificial Sequence: Synthetic oligonucleotide
  source 1..20: mol_type = other DNA
ggactagagaccacttaacc

SEQ ID NO: 117 - DNA, 20 bp, synthetic construct
  misc_feature 1..20: note = Description of Artificial Sequence: Synthetic oligonucleotide
  source 1..20: mol_type = other DNA
gaccacttaacctcccccta

SEQ ID NO: 118 - DNA, 20 bp, synthetic construct
  misc_feature 1..20: note = Description of Artificial Sequence: Synthetic oligonucleotide
  source 1..20: mol_type = other DNA
gagtaccctccaaagaattt

SEQ ID NO: 119 - DNA, 20 bp, synthetic construct
  misc_feature 1..20: note = Description of Artificial Sequence: Synthetic oligonucleotide
  source 1..20: mol_type = other DNA
atggaagcctgacccctata

SEQ ID NO: 120 - DNA, 20 bp, synthetic construct
  misc_feature 1..20: note = Description of Artificial Sequence: Synthetic oligonucleotide
  source 1..20: mol_type = other DNA
gccctgaactagataaacac

SEQ ID NO: 121 - DNA, 20 bp, synthetic construct
  misc_feature 1..20: note = Description of Artificial Sequence: Synthetic oligonucleotide
  source 1..20: mol_type = other DNA
gccctttggtccaacctata

SEQ ID NO: 122 - DNA, 20 bp, synthetic construct
  misc_feature 1..20: note = Description of Artificial Sequence: Synthetic oligonucleotide
  source 1..20: mol_type = other DNA
ccctttggtccaacctataa

SEQ ID NO: 123 - DNA, 20 bp, synthetic construct
  misc_feature 1..20: note = Description of Artificial Sequence: Synthetic oligonucleotide
  source 1..20: mol_type = other DNA
gacagtaaaggctccacctg

SEQ ID NO: 124 - DNA, 20 bp, synthetic construct
  misc_feature 1..20: note = Description of Artificial Sequence: Synthetic oligonucleotide
  source 1..20: mol_type = other DNA
tcaacaagaagcctactgac

SEQ ID NO: 125 - DNA, 20 bp, synthetic construct
  misc_feature 1..20: note = Description of Artificial Sequence: Synthetic oligonucleotide
  source 1..20: mol_type = other DNA
atcataagagccccaatcat

SEQ ID NO: 126 - DNA, 20 bp, synthetic construct
  misc_feature 1..20: note = Description of Artificial Sequence: Synthetic oligonucleotide
  source 1..20: mol_type = other DNA
cataagagccccaatcatct

SEQ ID NO: 127 - DNA, 20 bp, synthetic construct
  misc_feature 1..20: note = Description of Artificial Sequence: Synthetic oligonucleotide
  source 1..20: mol_type = other DNA
tgatctgatagccaactcaa

SEQ ID NO: 128 - DNA, 20 bp, synthetic construct
  misc_feature 1..20: note = Description of Artificial Sequence: Synthetic oligonucleotide
  source 1..20: mol_type = other DNA
tcctgagtggtaatcagtcc

SEQ ID NO: 129 - DNA, 20 bp, synthetic construct
  misc_feature 1..20: note = Description of Artificial Sequence: Synthetic oligonucleotide
  source 1..20: mol_type = other DNA
gacctactacaaactatcag

SEQ ID NO: 130 - DNA, 20 bp, synthetic construct
  misc_feature 1..20: note = Description of Artificial Sequence: Synthetic oligonucleotide
  source 1..20: mol_type = other DNA
agaccaggttcaatagaatc

SEQ ID NO: 131 - DNA, 20 bp, synthetic construct
  misc_feature 1..20: note = Description of Artificial Sequence: Synthetic oligonucleotide
  source 1..20: mol_type = other DNA
agtaccaggaccagtacaac

SEQ ID NO: 132 - DNA, 20 bp, synthetic construct
  misc_feature 1..20: note = Description of Artificial Sequence: Synthetic oligonucleotide
  source 1..20: mol_type = other DNA
agtccaatacccttacctaa

SEQ ID NO: 133 - DNA, 20 bp, synthetic construct
  misc_feature 1..20: note = Description of Artificial Sequence: Synthetic oligonucleotide
  source 1..20: mol_type = other DNA
gcatgttagatccaaatccc

SEQ ID NO: 134 - DNA, 20 bp, synthetic construct
  misc_feature 1..20: note = Description of Artificial Sequence: Synthetic oligonucleotide
  source 1..20: mol_type = other DNA
gcccaatagcaagcagccta

SEQ ID NO: 135 - DNA, 20 bp, synthetic construct
  misc_feature 1..20: note = Description of Artificial Sequence: Synthetic oligonucleotide
  source 1..20: mol_type = other DNA
gactgactaaaggaggagcc

SEQ ID NO: 136 - DNA, 20 bp, synthetic construct
  misc_feature 1..20: note = Description of Artificial Sequence: Synthetic oligonucleotide
  source 1..20: mol_type = other DNA
caaccagtatcaataccctc

SEQ ID NO: 137 - DNA, 20 bp, synthetic construct
  misc_feature 1..20: note = Description of Artificial Sequence: Synthetic oligonucleotide
  source 1..20: mol_type = other DNA
tggatagaggattcaaggct

SEQ ID NO: 138 - DNA, 20 bp, synthetic construct
  misc_feature 1..20: note = Description of Artificial Sequence: Synthetic oligonucleotide
  source 1..20: mol_type = other DNA
gtcccagcaaatcaactacc

SEQ ID NO: 139 - DNA, 20 bp, synthetic construct
  misc_feature 1..20: note = Description of Artificial Sequence: Synthetic oligonucleotide
  source 1..20: mol_type = other DNA
gtagttaccacctaaacaga

SEQ ID NO: 140 - DNA, 20 bp, synthetic construct
  misc_feature 1..20: note = Description of Artificial Sequence: Synthetic oligonucleotide
  source 1..20: mol_type = other DNA
gatcaccagactcaagaacc

SEQ ID NO: 141 - DNA, 20 bp, synthetic construct
  misc_feature 1..20: note = Description of Artificial Sequence: Synthetic oligonucleotide
  source 1..20: mol_type = other DNA
gcagcaacctacctgacata

SEQ ID NO: 142 - DNA, 20 bp, synthetic construct
  misc_feature 1..20: note = Description of Artificial Sequence: Synthetic oligonucleotide
  source 1..20: mol_type = other DNA
agcaacctacctgacatacc

SEQ ID NO: 143 - DNA, 20 bp, synthetic construct
  misc_feature 1..20: note = Description of Artificial Sequence: Synthetic oligonucleotide
  source 1..20: mol_type = other DNA
gcaacctacctgacatacct

SEQ ID NO: 144 - DNA, 20 bp, synthetic construct
  misc_feature 1..20: note = Description of Artificial Sequence: Synthetic oligonucleotide
  source 1..20: mol_type = other DNA
gacaccctttggagtgtcta

SEQ ID NO: 145 - DNA, 20 bp, synthetic construct
  misc_feature 1..20: note = Description of Artificial Sequence: Synthetic oligonucleotide
  source 1..20: mol_type = other DNA
ccttagctgattgcccctta

SEQ ID NO: 146 - DNA, 20 bp, synthetic construct
  misc_feature 1..20: note = Description of Artificial Sequence: Synthetic oligonucleotide
  source 1..20: mol_type = other DNA
taccatataggaaacctcct

SEQ ID NO: 147 - DNA, 20 bp, synthetic construct
  misc_feature 1..20: note = Description of Artificial Sequence: Synthetic oligonucleotide
  source 1..20: mol_type = other DNA
ctatactactagaagaaccc

SEQ ID NO: 148 - DNA, 20 bp, synthetic construct
  misc_feature 1..20: note = Description of Artificial Sequence: Synthetic oligonucleotide
  source 1..20: mol_type = other DNA
gtgcccccaaacaagaagtt

SEQ ID NO: 149 - DNA, 20 bp, synthetic construct
  misc_feature 1..20: note = Description of Artificial Sequence: Synthetic oligonucleotide
  source 1..20: mol_type = other DNA
gtacacaaacatccctctaa

SEQ ID NO: 150 - DNA, 20 bp, synthetic construct
  misc_feature 1..20: note = Description of Artificial Sequence: Synthetic oligonucleotide
  source 1..20: mol_type = other DNA
ccatattacaccttaggaac

SEQ ID NO: 151 - DNA, 20 bp, synthetic construct
  misc_feature 1..20: note = Description of Artificial Sequence: Synthetic oligonucleotide
  source 1..20: mol_type = other DNA
ccccaacttgttacctaagc

SEQ ID NO: 152 - DNA, 20 bp, synthetic construct
  misc_feature 1..20: note = Description of Artificial Sequence: Synthetic oligonucleotide
  source 1..20: mol_type = other DNA
ccaacttgttacctaagcac

SEQ ID NO: 153 - DNA, 20 bp, synthetic construct
  misc_feature 1..20: note = Description of Artificial Sequence: Synthetic oligonucleotide
  source 1..20: mol_type = other DNA
gcctaaccagtggagtacta

SEQ ID NO: 154 - DNA, 20 bp, synthetic construct
  misc_feature 1..20: note = Description of Artificial Sequence: Synthetic oligonucleotide
  source 1..20: mol_type = other DNA
accagtggagtactatggac

SEQ ID NO: 155 - DNA, 20 bp, synthetic construct
  misc_feature 1..20: note = Description of Artificial Sequence: Synthetic oligonucleotide
  source 1..20: mol_type = other DNA
gtataccatccacctgagtt

SEQ ID NO: 156 - DNA, 20 bp, synthetic construct
  misc_feature 1..20: note = Description of Artificial Sequence: Synthetic oligonucleotide
  source 1..20: mol_type = other DNA
gtggcaaaagttcactactc

SEQ ID NO: 157 - DNA, 20 bp, synthetic construct
  misc_feature 1..20: note = Description of Artificial Sequence: Synthetic oligonucleotide
  source 1..20: mol_type = other DNA
ggtactcttcagatacctaa

SEQ ID NO: 158 - DNA, 20 bp, synthetic construct
  misc_feature 1..20: note = Description of Artificial Sequence: Synthetic oligonucleotide
  source 1..20: mol_type = other DNA
gtacctgaatcaagacccac

SEQ ID NO: 159 - DNA, 20 bp, synthetic construct
  misc_feature 1..20: note = Description of Artificial Sequence: Synthetic oligonucleotide
  source 1..20: mol_type = other DNA
tggtaaggcagtcccaacaa

SEQ ID NO: 160 - DNA, 20 bp, synthetic construct
  misc_feature 1..20: note = Description of Artificial Sequence: Synthetic oligonucleotide
  source 1..20: mol_type = other DNA
acaggcatagcttatggatc

SEQ ID NO: 161 - DNA, 20 bp, synthetic construct
  misc_feature 1..20: note = Description of Artificial Sequence: Synthetic oligonucleotide
  source 1..20: mol_type = other DNA
aggcatagcttatggatcaa

SEQ ID NO: 162 - DNA, 20 bp, synthetic construct
  misc_feature 1..20: note = Description of Artificial Sequence: Synthetic oligonucleotide
  source 1..20: mol_type = other DNA
tagcaccctaaaccatcaat

SEQ ID NO: 163 - DNA, 20 bp, synthetic construct
  misc_feature 1..20: note = Description of Artificial Sequence: Synthetic oligonucleotide
  source 1..20: mol_type = other DNA
agcaagtagttacccttgag

SEQ ID NO: 164 - DNA, 20 bp, synthetic construct
  misc_feature 1..20: note = Description of Artificial Sequence: Synthetic oligonucleotide
  source 1..20: mol_type = other DNA
tccactattaaggatcttcc

SEQ ID NO: 165 - DNA, 20 bp, synthetic construct
  misc_feature 1..20: note = Description of Artificial Sequence: Synthetic oligonucleotide
  source 1..20: mol_type = other DNA
gctgccacctagaattaggt

SEQ ID NO: 166 - DNA, 20 bp, synthetic construct
  misc_feature 1..20: note = Description of Artificial Sequence: Synthetic oligonucleotide
  source 1..20: mol_type = other DNA
ttagcacaggtatccctaca

SEQ ID NO: 167 - DNA, 20 bp, synthetic construct
  misc_feature 1..20: note = Description of Artificial Sequence: Synthetic oligonucleotide
  source 1..20: mol_type = other DNA
tagcctgtagccctccccta

SEQ ID NO: 168 - DNA, 20 bp, synthetic construct
  misc_feature 1..20: note = Description of Artificial Sequence: Synthetic oligonucleotide
  source 1..20: mol_type = other DNA
gttggtaattccacccctcc

SEQ ID NO: 169 - DNA, 20 bp, synthetic construct
  misc_feature 1..20: note = Description of Artificial Sequence: Synthetic oligonucleotide
  source 1..20: mol_type = other DNA
cctacatccagttgattagc

SEQ ID NO: 170 - DNA, 20 bp, synthetic construct
  misc_feature 1..20: note = Description of Artificial Sequence: Synthetic oligonucleotide
  source 1..20: mol_type = other DNA
gattcacaggactagagacc

SEQ ID NO: 171 - DNA, 20 bp, synthetic construct
  misc_feature 1..20: note = Description of Artificial Sequence: Synthetic oligonucleotide
  source 1..20: mol_type = other DNA
aggtgtccaaactaccataa

SEQ ID NO: 172 - DNA, 20 bp, synthetic construct
  misc_feature 1..20: note = Description of Artificial Sequence: Synthetic oligonucleotide
  source 1..20: mol_type = other DNA
gtgtccaaactaccataaac

SEQ ID NO: 173 - DNA, 20 bp, synthetic construct
  misc_feature 1..20: note = Description of Artificial Sequence: Synthetic oligonucleotide
  source 1..20: mol_type = other DNA
cctaaccagtcagctattag

SEQ ID NO: 174 - DNA, 20 bp, synthetic construct
  misc_feature 1..20: note = Description of Artificial Sequence: Synthetic oligonucleotide
  source 1..20: mol_type = other DNA
gatccctttccatgtactag

SEQ ID NO: 175 - DNA, 20 bp, synthetic construct
  misc_feature 1..20: note = Description of Artificial Sequence: Synthetic oligonucleotide
  source 1..20: mol_type = other DNA
gcctagcatatattacccca

SEQ ID NO: 176 - DNA, 20 bp, synthetic construct
  misc_feature 1..20: note = Description of Artificial Sequence: Synthetic oligonucleotide
  source 1..20: mol_type = other DNA
ggagaccactatattatccc

SEQ ID NO: 177 - DNA, 20 bp, synthetic construct
  misc_feature 1..20: note = Description of Artificial Sequence: Synthetic oligonucleotide
  source 1..20: mol_type = other DNA
gacaaccagcagaatcccta

SEQ ID NO: 178 - DNA, 20 bp, synthetic construct
  misc_feature 1..20: note = Description of Artificial Sequence: Synthetic oligonucleotide
  source 1..20: mol_type = other DNA
ggtactatatccaactggac

SEQ ID NO: 179 - DNA, 20 bp, synthetic construct
  misc_feature 1..20: note = Description of Artificial Sequence: Synthetic oligonucleotide
  source 1..20: mol_type = other DNA
cattaggtatggcatgatcc

SEQ ID NO: 180 - DNA, 20 bp, synthetic construct
  misc_feature 1..20: note = Description of Artificial Sequence: Synthetic oligonucleotide
  source 1..20: mol_type = other DNA
gaacatcacttaaatggtcc

SEQ ID NO: 181 - DNA, 20 bp, synthetic construct
  misc_feature 1..20: note = Description of Artificial Sequence: Synthetic oligonucleotide
  source 1..20: mol_type = other DNA
agccctttggtccaacctat

SEQ ID NO: 182 - DNA, 20 bp, synthetic construct
  misc_feature 1..20: note = Description of Artificial Sequence: Synthetic oligonucleotide
  source 1..20: mol_type = other DNA
gtatgcccaccaaagttgtc

SEQ ID NO: 183 - DNA, 20 bp, synthetic construct
  misc_feature 1..20: note = Description of Artificial Sequence: Synthetic oligonucleotide
  source 1..20: mol_type = other DNA
ataccaatggcaaggtttgg

SEQ ID NO: 184 - DNA, 20 bp, synthetic construct
  misc_feature 1..20: note = Description of Artificial Sequence: Synthetic oligonucleotide
  source 1..20: mol_type = other DNA
gcaagtggcagcccaataat

SEQ ID NO: 185 - DNA, 20 bp, synthetic construct
  misc_feature 1..20: note = Description of Artificial Sequence: Synthetic oligonucleotide
  source 1..20: mol_type = other DNA
gccaataatcacacccttgg

SEQ ID NO: 186 - DNA, 20 bp, synthetic construct
  misc_feature 1..20: note = Description of Artificial Sequence: Synthetic oligonucleotide
  source 1..20: mol_type = other DNA
acagatcccttacactatca

SEQ ID NO: 187 - DNA, 20 bp, synthetic construct
  misc_feature 1..20: note = Description of Artificial Sequence: Synthetic oligonucleotide
  source 1..20: mol_type = other DNA
atcccatacagccagtttgg

SEQ ID NO: 188 - DNA, 20 bp, synthetic construct
  misc_feature 1..20: note = Description of Artificial Sequence: Synthetic oligonucleotide
  source 1..20: mol_type = other DNA
gcaaccaaggtactctgaac

SEQ ID NO: 189 - DNA, 20 bp, synthetic construct
  misc_feature 1..20: note = Description of Artificial Sequence: Synthetic oligonucleotide
  source 1..20: mol_type = other DNA
taagtccttggtcaacttgc

SEQ ID NO: 190 - DNA, 20 bp, synthetic construct
  misc_feature 1..20: note = Description of Artificial Sequence: Synthetic oligonucleotide
  source 1..20: mol_type = other DNA
gatgccacctaaattgctgg

SEQ ID NO: 191 - DNA, 20 bp, synthetic construct
  misc_feature 1..20: note = Description of Artificial Sequence: Synthetic oligonucleotide
  source 1..20: mol_type = other DNA
ggattcccagtttaagtcaa

SEQ ID NO: 192 - DNA, 20 bp, synthetic construct
  misc_feature 1..20: note = Description of Artificial Sequence: Synthetic oligonucleotide
  source 1..20: mol_type = other DNA
gaaaggttatctgttgtgcc

SEQ ID NO: 193 - DNA, 20 bp, synthetic construct
  misc_feature 1..20: note = Description of Artificial Sequence: Synthetic oligonucleotide
  source 1..20: mol_type = other DNA
gactccttatagtcactacc

SEQ ID NO: 194 - DNA, 20 bp, synthetic construct
  misc_feature 1..20: note = Description of Artificial Sequence: Synthetic oligonucleotide
  source 1..20: mol_type = other DNA
taccaggaccagtacaacct

SEQ ID NO: 195 - DNA, 20 bp, synthetic construct
  misc_feature 1..20: note = Description of Artificial Sequence: Synthetic oligonucleotide
  source 1..20: mol_type = other DNA
gaccagtacaacctgtagta

SEQ ID NO: 196 - DNA, 20 bp, synthetic construct
  misc_feature 1..20: note = Description of Artificial Sequence: Synthetic oligonucleotide
  source 1..20: mol_type = other DNA
ggtacaaaaggttccagtag

SEQ ID NO: 197 - DNA, 20 bp, synthetic construct
  misc_feature 1..20: note = Description of Artificial Sequence: Synthetic oligonucleotide
  source 1..20: mol_type = other DNA
gtacaaaaggttccagtagc

SEQ ID NO: 198 - DNA, 20 bp, synthetic construct
  misc_feature 1..20: note = Description of Artificial Sequence: Synthetic oligonucleotide
  source 1..20: mol_type = other DNA
gcttcacatagtccaatacc

SEQ ID NO: 199 - DNA, 20 bp, synthetic construct
  misc_feature 1..20: note = Description of Artificial Sequence: Synthetic oligonucleotide
  source 1..20: mol_type = other DNA
gtccaatacccttacctaat

SEQ ID NO: 200 - DNA, 20 bp, synthetic construct
  misc_feature 1..20: note = Description of Artificial Sequence: Synthetic oligonucleotide
  source 1..20: mol_type = other DNA
tacctaatcaagagaaggtc

SEQ ID NO: 201 - DNA, 20 bp, synthetic construct
  misc_feature 1..20: note = Description of Artificial Sequence: Synthetic oligonucleotide
  source 1..20: mol_type = other DNA
tgaaagtagttggactctcc

SEQ ID NO: 202 - DNA, 20 bp, synthetic construct
  misc_feature 1..20: note = Description of Artificial Sequence: Synthetic oligonucleotide
  source 1..20: mol_type = other DNA
aaagtagttggactctccca

SEQ ID NO: 203 - DNA, 20 bp, synthetic construct
  misc_feature 1..20: note = Description of Artificial Sequence: Synthetic oligonucleotide
  source 1..20: mol_type = other DNA
acctaagcccacctacaata

SEQ ID NO: 204 - DNA, 20 bp, synthetic construct
  misc_feature 1..20: note = Description of Artificial Sequence: Synthetic oligonucleotide
  source 1..20: mol_type = other DNA
actaagttcagctaccacca

SEQ ID NO: 205 - DNA, 20 bp, synthetic construct
  misc_feature 1..20: note = Description of Artificial Sequence: Synthetic oligonucleotide
  source 1..20: mol_type = other DNA
aacccccctgctaagagcca

SEQ ID NO: 206 - DNA, 20 bp, synthetic construct
  misc_feature 1..20: note = Description of Artificial Sequence: Synthetic oligonucleotide
  source 1..20: mol_type = other DNA
gcactgtggctattacaccc

SEQ ID NO: 207 - DNA, 20 bp, synthetic construct
  misc_feature 1..20: note = Description of Artificial Sequence: Synthetic oligonucleotide
  source 1..20: mol_type = other DNA
accatgatgctaccctcagt

SEQ ID NO: 208 - DNA, 20 bp, synthetic construct
  misc_feature 1..20: note = Description of Artificial Sequence: Synthetic oligonucleotide
  source 1..20: mol_type = other DNA
gccccttgagtgctgtttat

SEQ ID NO: 209 - DNA, 20 bp, synthetic construct
  misc_feature 1..20: note = Description of Artificial Sequence: Synthetic oligonucleotide
  source 1..20: mol_type = other DNA
accaggagcatttgttgatc

SEQ ID NO: 210 - DNA, 20 bp, synthetic construct
  misc_feature 1..20: note = Description of Artificial Sequence: Synthetic oligonucleotide
  source 1..20: mol_type = other DNA
atgctacaacccaactcccc

SEQ ID NO: 211 - DNA, 20 bp, synthetic construct
  misc_feature 1..20: note = Description of Artificial Sequence: Synthetic oligonucleotide
  source 1..20: mol_type = other DNA
gctgattgccccttaacaga

SEQ ID NO: 212 - DNA, 20 bp, synthetic construct
  misc_feature 1..20: note = Description of Artificial Sequence: Synthetic oligonucleotide
  source 1..20: mol_type = other DNA
gactgctaaggctactacaa

SEQ ID NO: 213 - DNA, 20 bp, synthetic construct
  misc_feature 1..20: note = Description of Artificial Sequence: Synthetic oligonucleotide
  source 1..20: mol_type = other DNA
gtgaggatcatgtaacagtc

SEQ ID NO: 214 - DNA, 20 bp, synthetic construct
  misc_feature 1..20: note = Description of Artificial Sequence: Synthetic oligonucleotide
  source 1..20: mol_type = other DNA
agtttagacttgccacacta

SEQ ID NO: 215 - DNA, 20 bp, synthetic construct
  misc_feature 1..20: note = Description of Artificial Sequence: Synthetic oligonucleotide
  source 1..20: mol_type = other DNA
gtgtacctaaatacaggtcc

SEQ ID NO: 216 - DNA, 20 bp, synthetic construct
  misc_feature 1..20: note = Description of Artificial Sequence: Synthetic oligonucleotide
  source 1..20: mol_type = other DNA
tgcccactgaaaccttactc

SEQ ID NO: 217 - DNA, 20 bp, synthetic construct
  misc_feature 1..20: note = Description of Artificial Sequence: Synthetic oligonucleotide
  source 1..20: mol_type = other DNA
tatgcccatccctaagttgt

SEQ ID NO: 218 - DNA, 20 bp, synthetic construct
  misc_feature 1..20: note = Description of Artificial Sequence: Synthetic oligonucleotide
  source 1..20: mol_type = other DNA
ggactatctctaatcagtgg

SEQ ID NO: 219 - DNA, 20 bp, synthetic construct
  misc_feature 1..20: note = Description of Artificial Sequence: Synthetic oligonucleotide
  source 1..20: mol_type = other DNA
aaggtgccccaacttgttac

SEQ ID NO: 220 - DNA, 20 bp, synthetic construct
  misc_feature 1..20: note = Description of Artificial Sequence: Synthetic oligonucleotide
  source 1..20: mol_type = other DNA
tcattgccttacctaagtac

SEQ ID NO: 221 - DNA, 20 bp, synthetic construct
  misc_feature 1..20: note = Description of Artificial Sequence: Synthetic oligonucleotide
  source 1..20: mol_type = other DNA
cattgccttacctaagtaca

SEQ ID NO: 222 - DNA, 20 bp, synthetic construct
  misc_feature 1..20: note = Description of Artificial Sequence: Synthetic oligonucleotide
  source 1..20: mol_type = other DNA
cagtggagtactatggacta

SEQ ID NO: 223 - DNA, 20 bp, synthetic construct
  misc_feature 1..20: note = Description of Artificial Sequence: Synthetic oligonucleotide
  source 1..20: mol_type = other DNA
ggagtactatggactaagaa

SEQ ID NO: 224 - DNA, 20 bp, synthetic construct
  misc_feature 1..20: note = Description of Artificial Sequence: Synthetic oligonucleotide
  source 1..20: mol_type = other DNA
taagccctaagtatatcctc

SEQ ID NO: 225 - DNA, 20 bp, synthetic construct
  misc_feature 1..20: note = Description of Artificial Sequence: Synthetic oligonucleotide
  source 1..20: mol_type = other DNA
aagttcactactcccaacta

SEQ ID NO: 226 - DNA, 20 bp, synthetic construct
  misc_feature 1..20: note = Description of Artificial Sequence: Synthetic oligonucleotide
  source 1..20: mol_type = other DNA
ggttggcacatcacactatt

SEQ ID NO: 227 - DNA, 20 bp, synthetic construct
  misc_feature 1..20: note = Description of Artificial Sequence: Synthetic oligonucleotide
  source 1..20: mol_type = other DNA
gtttgcttaaccaatgctgg

SEQ ID NO: 228 - DNA, 20 bp, synthetic construct
  misc_feature 1..20: note = Description of Artificial Sequence: Synthetic oligonucleotide
  source 1..20: mol_type = other DNA
acagtttgcctaaacctggc

SEQ ID NO: 229 - DNA, 20 bp, synthetic construct
  misc_feature 1..20: note = Description of Artificial Sequence: Synthetic oligonucleotide
  source 1..20: mol_type = other DNA
acacaatcccatgataggac

SEQ ID NO: 230 - DNA, 20 bp, synthetic construct
  misc_feature 1..20: note = Description of Artificial Sequence: Synthetic oligonucleotide
  source 1..20: mol_type = other DNA
cccccaatctagttcaagca

SEQ ID NO: 231 - DNA, 20 bp, synthetic construct
  misc_feature 1..20: note = Description of Artificial Sequence: Synthetic oligonucleotide
  source 1..20: mol_type = other DNA
gactacctcctacttttagt

SEQ ID NO: 232 - DNA, 20 bp, synthetic construct
  misc_feature 1..20: note = Description of Artificial Sequence: Synthetic oligonucleotide
  source 1..20: mol_type = other DNA
aagtacctgaatcaagaccc

SEQ ID NO: 233 - DNA, 20 bp, synthetic construct
  misc_feature 1..20: note = Description of Artificial Sequence: Synthetic oligonucleotide
  source 1..20: mol_type = other DNA
ctactggtaaggcagtccca

SEQ ID NO: 234 - DNA, 20 bp, synthetic construct
  misc_feature 1..20: note = Description of Artificial Sequence: Synthetic oligonucleotide
  source 1..20: mol_type = other DNA
ggtaaggcagtcccaacaaa

SEQ ID NO: 235 - DNA, 20 bp, synthetic construct
  misc_feature 1..20: note = Description of Artificial Sequence: Synthetic oligonucleotide
  source 1..20: mol_type = other DNA
ttaacagcaagtagttaccc

SEQ ID NO: 236 - DNA, 20 bp, synthetic construct
  misc_feature 1..20: note = Description of Artificial Sequence: Synthetic oligonucleotide
  source 1..20: mol_type = other DNA
tcacactaacctaaggtaac

SEQ ID NO: 237 - DNA, 20 bp, synthetic construct
  misc_feature 1..20: note = Description of Artificial Sequence: Synthetic oligonucleotide
  source 1..20: mol_type = other DNA
gatctaagagttaagctctc

SEQ ID NO: 238 - DNA, 20 bp, synthetic construct
  misc_feature 1..20: note = Description of Artificial Sequence: Synthetic oligonucleotide
  source 1..20: mol_type = other DNA
tagaatacttgacccatctc

SEQ ID NO: 239 - DNA, 20 bp, synthetic construct
  misc_feature 1..20: note = Description of Artificial Sequence: Synthetic oligonucleotide
  source 1..20: mol_type = other DNA
gcaaccctatgtaagtctat

SEQ ID NO: 240 - DNA, 20 bp, synthetic construct
  misc_feature 1..20: note = Description of Artificial Sequence: Synthetic oligonucleotide
  source 1..20: mol_type = other DNA
ggctgccacctagaattagg

SEQ ID NO: 241 - DNA, 20 bp, synthetic construct
  misc_feature 1..20: note = Description of Artificial Sequence: Synthetic oligonucleotide
  source 1..20: mol_type = other DNA
gtatccctacacttctacac

SEQ ID NO: 242 - DNA, 20 bp, synthetic construct
  misc_feature 1..20: note = Description of Artificial Sequence: Synthetic oligonucleotide
  source 1..20: mol_type = other DNA
aactgttcctcccagttggt

SEQ ID NO: 243 - DNA, 20 bp, synthetic construct
  misc_feature 1..20: note = Description of Artificial Sequence: Synthetic oligonucleotide
  source 1..20: mol_type = other DNA
ccagttggtaattccacccc

SEQ ID NO: 244 - DNA, 20 bp, synthetic construct
  misc_feature 1..20: note = Description of Artificial Sequence: Synthetic oligonucleotide
  source 1..20: mol_type = other DNA
ttggtaattccacccctcca

SEQ ID NO: 245 - DNA, 20 bp, synthetic construct
  misc_feature 1..20: note = Description of Artificial Sequence: Synthetic oligonucleotide
  source 1..20: mol_type = other DNA
ggtcaaagacctgagtcacc

SEQ ID NO: 246 - DNA, 20 bp, synthetic construct
  misc_feature 1..20: note = Description of Artificial Sequence: Synthetic oligonucleotide
  source 1..20: mol_type = other DNA
actcatggagtattactgcc

SEQ ID NO: 247 - DNA, 20 bp, synthetic construct
  misc_feature 1..20: note = Description of Artificial Sequence: Synthetic oligonucleotide
  source 1..20: mol_type = other DNA
atggagtattactgccccaa

SEQ ID NO: 248 - DNA, 20 bp, synthetic construct
  misc_feature 1..20: note = Description of Artificial Sequence: Synthetic oligonucleotide
  source 1..20: mol_type = other DNA
aacctccccctacatcctat

SEQ ID NO: 249 - DNA, 20 bp, synthetic construct
  misc_feature 1..20: note = Description of Artificial Sequence: Synthetic oligonucleotide
  source 1..20: mol_type = other DNA
cccatcacatcaagttacag

SEQ ID NO: 250 - DNA, 20 bp, synthetic construct
  misc_feature 1..20: note = Description of Artificial Sequence: Synthetic oligonucleotide
  source 1..20: mol_type = other DNA
tatgacaatcaatcccaccc

SEQ ID NO: 251 - DNA, 20 bp, synthetic construct
  misc_feature 1..20: note = Description of Artificial Sequence: Synthetic oligonucleotide
  source 1..20: mol_type = other DNA
gaaattcccctacccagtcc

SEQ ID NO: 252 - DNA, 20 bp, synthetic construct
  misc_feature 1..20: note = Description of Artificial Sequence: Synthetic oligonucleotide
  source 1..20: mol_type = other DNA
aattcccctacccagtccta

SEQ ID NO: 253 - DNA, 20 bp, synthetic construct
  misc_feature 1..20: note = Description of Artificial Sequence: Synthetic oligonucleotide
  source 1..20: mol_type = other DNA
ccctagacagtgtagtagtt

SEQ ID NO: 254 - DNA, 20 bp, synthetic construct
  misc_feature 1..20: note = Description of Artificial Sequence: Synthetic oligonucleotide
  source 1..20: mol_type = other DNA
ttccaagagttagcaccctc

SEQ ID NO: 255 - DNA, 20 bp, synthetic construct
  misc_feature 1..20: note = Description of Artificial Sequence: Synthetic oligonucleotide
  source 1..20: mol_type = other DNA
gtatatcccaatgataccag

SEQ ID NO: 256 - DNA, 20 bp, synthetic construct
  misc_feature 1..20: note = Description of Artificial Sequence: Synthetic oligonucleotide
  source 1..20: mol_type = other DNA
aaccagtcagctattagaac

SEQ ID NO: 257 - DNA, 20 bp, synthetic construct
  misc_feature 1..20: note = Description of Artificial Sequence: Synthetic oligonucleotide
  source 1..20: mol_type = other DNA
gccctgccatcaaaaaactc

SEQ ID NO: 258 - DNA, 20 bp, synthetic construct
  misc_feature 1..20: note = Description of Artificial Sequence: Synthetic oligonucleotide
  source 1..20: mol_type = other DNA
tcaggatctactcctactac

SEQ ID NO: 259 - DNA, 20 bp, synthetic construct
  misc_feature 1..20: note = Description of Artificial Sequence: Synthetic oligonucleotide
  source 1..20: mol_type = other DNA
caggatctactcctactact

SEQ ID NO: 260 - DNA, 20 bp, synthetic construct
  misc_feature 1..20: note = Description of Artificial Sequence: Synthetic oligonucleotide
  source 1..20: mol_type = other DNA
gagccacttacagatgatcc

SEQ ID NO: 261 - DNA, 20 bp, synthetic construct
  misc_feature 1..20: note = Description of Artificial Sequence: Synthetic oligonucleotide
  source 1..20: mol_type = other DNA
gttattagacacctactctc

SEQ ID NO: 262 - DNA, 20 bp, synthetic construct
  misc_feature 1..20: note = Description of Artificial Sequence: Synthetic oligonucleotide
  source 1..20: mol_type = other DNA
ctctgtagtagtttaggtgg

SEQ ID NO: 263 - DNA, 20 bp, synthetic construct
  misc_feature 1..20: note = Description of Artificial Sequence: Synthetic oligonucleotide
  source 1..20: mol_type = other DNA
ggaagcctgacccctatact

SEQ ID NO: 264 - DNA, 20 bp, synthetic construct
  misc_feature 1..20: note = Description of Artificial Sequence: Synthetic oligonucleotide
  source 1..20: mol_type = other DNA
gacccctatacttgacagaa

SEQ ID NO: 265 - DNA, 20 bp, synthetic construct
  misc_feature 1..20: note = Description of Artificial Sequence: Synthetic oligonucleotide
  source 1..20: mol_type = other DNA
ctgccctgaactagataaac

SEQ ID NO: 266 - DNA, 20 bp, synthetic construct
  misc_feature 1..20: note = Description of Artificial Sequence: Synthetic oligonucleotide
  source 1..20: mol_type = other DNA
gctaaaactccaatcctatc

SEQ ID NO: 267 - DNA, 20 bp, synthetic construct
  misc_feature 1..20: note = Description of Artificial Sequence: Synthetic oligonucleotide
  source 1..20: mol_type = other DNA
cctattagcttaaacccatc

SEQ ID NO: 268 - DNA, 20 bp, synthetic construct
  misc_feature 1..20: note = Description of Artificial Sequence: Synthetic oligonucleotide
  source 1..20: mol_type = other DNA
accatttgctagataggtgc

SEQ ID NO: 269 - DNA, 20 bp, synthetic construct
  misc_feature 1..20: note = Description of Artificial Sequence: Synthetic oligonucleotide
  source 1..20: mol_type = other DNA
ggcagcccaataatggagtt

SEQ ID NO: 270 - DNA, 20 bp, synthetic construct
  misc_feature 1..20: note = Description of Artificial Sequence: Synthetic oligonucleotide
  source 1..20: mol_type = other DNA
gcagcccaataatggagttg

SEQ ID NO: 271 - DNA, 20 bp, synthetic construct
  misc_feature 1..20: note = Description of Artificial Sequence: Synthetic oligonucleotide
  source 1..20: mol_type = other DNA
acaactatgatctgatagcc

SEQ ID NO: 272 - DNA, 20 bp, synthetic construct
  misc_feature 1..20: note = Description of Artificial Sequence: Synthetic oligonucleotide
  source 1..20: mol_type = other DNA
ggaagctagttatacaacac

SEQ ID NO: 273 - DNA, 20 bp, synthetic construct
  misc_feature 1..20: note = Description of Artificial Sequence: Synthetic oligonucleotide
  source 1..20: mol_type = other DNA
atcagtcctattaacctacc

SEQ ID NO: 274 - DNA, 20 bp, synthetic construct
  misc_feature 1..20: note = Description of Artificial Sequence: Synthetic oligonucleotide
  source 1..20: mol_type = other DNA
ggttaacttagcttggtctc

SEQ ID NO: 275 - DNA, 20 bp, synthetic construct
  misc_feature 1..20: note = Description of Artificial Sequence: Synthetic oligonucleotide
  source 1..20: mol_type = other DNA
gacaggattcccagtttaag

SEQ ID NO: 276 - DNA, 20 bp, synthetic construct
  misc_feature 1..20: note = Description of Artificial Sequence: Synthetic oligonucleotide
  source 1..20: mol_type = other DNA
acttgagtaccaggaccagt

SEQ ID NO: 277 - DNA, 20 bp, synthetic construct
  misc_feature 1..20: note = Description of Artificial Sequence: Synthetic oligonucleotide
  source 1..20: mol_type = other DNA
cttcacatagtccaataccc

SEQ ID NO: 278 - DNA, 20 bp, synthetic construct
  misc_feature 1..20: note = Description of Artificial Sequence: Synthetic oligonucleotide
  source 1..20: mol_type = other DNA
ttcacatagtccaataccct

SEQ ID NO: 279 - DNA, 20 bp, synthetic construct
  misc_feature 1..20: note = Description of Artificial Sequence: Synthetic oligonucleotide
  source 1..20: mol_type = other DNA
catagtccaatacccttacc

SEQ ID NO: 280 - DNA, 20 bp, synthetic construct
  misc_feature 1..20: note = Description of Artificial Sequence: Synthetic oligonucleotide
  source 1..20: mol_type = other DNA
tagtccaatacccttaccta

SEQ ID NO: 281 - DNA, 20 bp, synthetic construct
  misc_feature 1..20: note = Description of Artificial Sequence: Synthetic oligonucleotide
  source 1..20: mol_type = other DNA
ggctgctcaatgacaagtgg

SEQ ID NO: 282 - DNA, 20 bp, synthetic construct
  misc_feature 1..20: note = Description of Artificial Sequence: Synthetic oligonucleotide
  source 1..20: mol_type = other DNA
ggactaacccagaggtcacc

SEQ ID NO: 283 - DNA, 20 bp, synthetic construct
  misc_feature 1..20: note = Description of Artificial Sequence: Synthetic oligonucleotide
  source 1..20: mol_type = other DNA
gagtctgcctattcctgatc

SEQ ID NO: 284 - DNA, 20 bp, synthetic construct
  misc_feature 1..20: note = Description of Artificial Sequence: Synthetic oligonucleotide
  source 1..20: mol_type = other DNA
agtagttggactctcccagt

SEQ ID NO: 285 - DNA, 20 bp, synthetic construct
  misc_feature 1..20: note = Description of Artificial Sequence: Synthetic oligonucleotide
  source 1..20: mol_type = other DNA
gttcctacagtttaacacag

SEQ ID NO: 286 - DNA, 20 bp, synthetic construct
  misc_feature 1..20: note = Description of Artificial Sequence: Synthetic oligonucleotide
  source 1..20: mol_type = other DNA
ggttccccaccaacagaatg

SEQ ID NO: 287 - DNA, 20 bp, synthetic construct
  misc_feature 1..20: note = Description of Artificial Sequence: Synthetic oligonucleotide
  source 1..20: mol_type = other DNA
tacctaagcccacctacaat

SEQ ID NO: 288 - DNA, 20 bp, synthetic construct
  misc_feature 1..20: note = Description of Artificial Sequence: Synthetic oligonucleotide
  source 1..20: mol_type = other DNA
accagtatcaataccctcaa

SEQ ID NO: 289 - DNA, 20 bp, synthetic construct
  misc_feature 1..20: note = Description of Artificial Sequence: Synthetic oligonucleotide
  source 1..20: mol_type = other DNA
gatgatctcagctagaatcc

SEQ ID NO: 290 - DNA, 20 bp, synthetic construct
  misc_feature 1..20: note = Description of Artificial Sequence: Synthetic oligonucleotide
  source 1..20: mol_type = other DNA
gatctcagctagaatcctta

SEQ ID NO: 291 - DNA, 20 bp, synthetic construct
  misc_feature 1..20: note = Description of Artificial Sequence: Synthetic oligonucleotide
  source 1..20: mol_type = other DNA
atggatagaggattcaaggc

SEQ ID NO: 292 - DNA, 20 bp, synthetic construct
  misc_feature 1..20: note = Description of Artificial Sequence: Synthetic oligonucleotide
  source 1..20: mol_type = other DNA
agccctaactgtatcttccc

SEQ ID NO: 293 - DNA, 20 bp, synthetic construct
  misc_feature 1..20: note = Description of Artificial Sequence: Synthetic oligonucleotide
  source 1..20: mol_type = other DNA
ctaagagtgatgacagttcc

SEQ ID NO: 294 - DNA, 20 bp, synthetic construct
  misc_feature 1..20: note = Description of Artificial Sequence: Synthetic oligonucleotide
  source 1..20: mol_type = other DNA
ggacacttaaacaggcacta

SEQ ID NO: 295 - DNA, 20 bp, synthetic construct
  misc_feature 1..20: note = Description of Artificial Sequence: Synthetic oligonucleotide
  source 1..20: mol_type = other DNA
gaaaaataacccccctgcta

SEQ ID NO: 296 - DNA, 20 bp, synthetic construct
  misc_feature 1..20: note = Description of Artificial Sequence: Synthetic oligonucleotide
  source 1..20: mol_type = other DNA
aaaataacccccctgctaag

SEQ ID NO: 297 - DNA, 20 bp, synthetic construct
  misc_feature 1..20: note = Description of Artificial Sequence: Synthetic oligonucleotide
  source 1..20: mol_type = other DNA
gactgactcctatccaacac

SEQ ID NO: 298 - DNA, 20 bp, synthetic construct
  misc_feature 1..20: note = Description of Artificial Sequence: Synthetic oligonucleotide
  source 1..20: mol_type = other DNA
caccctattatactcagagc

SEQ ID NO: 299 - DNA, 20 bp, synthetic construct
  misc_feature 1..20: note = Description of Artificial Sequence: Synthetic oligonucleotide
  source 1..20: mol_type = other DNA
gtttctagccccttgagtgc

SEQ ID NO: 300 - DNA, 20 bp, synthetic construct
  misc_feature 1..20: note = Description of Artificial Sequence: Synthetic oligonucleotide
  source 1..20: mol_type = other DNA
gtaccatataggaaacctcc

SEQ ID NO: 301 - DNA, 20 bp, synthetic construct
  misc_feature 1..20: note = Description of Artificial Sequence: Synthetic oligonucleotide
  source 1..20: mol_type = other DNA
accatataggaaacctcctc

SEQ ID NO: 302 - DNA, 20 bp, synthetic construct
  misc_feature 1..20: note = Description of Artificial Sequence: Synthetic oligonucleotide
  source 1..20: mol_type = other DNA
caagtttagacttgccacac

SEQ ID NO: 303 - DNA, 20 bp, synthetic construct
  misc_feature 1..20: note = Description of Artificial Sequence: Synthetic oligonucleotide
  source 1..20: mol_type = other DNA
cctgctgaagctatactact

SEQ ID NO: 304 - DNA, 20 bp, synthetic construct
  misc_feature 1..20: note = Description of Artificial Sequence: Synthetic oligonucleotide
  source 1..20: mol_type = other DNA
tactagaagaacccatgagc

SEQ ID NO: 305 - DNA, 20 bp, synthetic construct
  misc_feature 1..20: note = Description of Artificial Sequence: Synthetic oligonucleotide
  source 1..20: mol_type = other DNA
gatccaggattatcatacca

SEQ ID NO: 306 - DNA, 20 bp, synthetic construct
  misc_feature 1..20: note = Description of Artificial Sequence: Synthetic oligonucleotide
  source 1..20: mol_type = other DNA
gcttcacccttctaggacta

SEQ ID NO: 307 - DNA, 20 bp, synthetic construct
  misc_feature 1..20: note = Description of Artificial Sequence: Synthetic oligonucleotide
  source 1..20: mol_type = other DNA
gattgctcctaccactcttg

SEQ ID NO: 308 - DNA, 20 bp, synthetic construct
  misc_feature 1..20: note = Description of Artificial Sequence: Synthetic oligonucleotide
  source 1..20: mol_type = other DNA
ggttacttcacaaactccaa

SEQ ID NO: 309 - DNA, 20 bp, synthetic construct
  misc_feature 1..20: note = Description of Artificial Sequence: Synthetic oligonucleotide
  source 1..20: mol_type = other DNA
gctgaccaagtttatggacc

SEQ ID NO: 310 - DNA, 20 bp, synthetic construct
  misc_feature 1..20: note = Description of Artificial Sequence: Synthetic oligonucleotide
  source 1..20: mol_type = other DNA
ggtattacacactcagccta

SEQ ID NO: 311 - DNA, 20 bp, synthetic construct
  misc_feature 1..20: note = Description of Artificial Sequence: Synthetic oligonucleotide
  source 1..20: mol_type = other DNA
gtattacacactcagcctag

SEQ ID NO: 312 - DNA, 20 bp, synthetic construct
  misc_feature 1..20: note = Description of Artificial Sequence: Synthetic oligonucleotide
  source 1..20: mol_type = other DNA
gtctccttaataccccaggt

SEQ ID NO: 313 - DNA, 20 bp, synthetic construct
  misc_feature 1..20: note = Description of Artificial Sequence: Synthetic oligonucleotide
  source 1..20: mol_type = other DNA
cttccataactaaggtgccc

SEQ ID NO: 314 - DNA, 20 bp, synthetic construct
  misc_feature 1..20: note = Description of Artificial Sequence: Synthetic oligonucleotide
  source 1..20: mol_type = other DNA
ggtgccccaacttgttacct

SEQ ID NO: 315 - DNA, 20 bp, synthetic construct
  misc_feature 1..20: note = Description of Artificial Sequence: Synthetic oligonucleotide
  source 1..20: mol_type = other DNA
cccctgttctctaatgtact

SEQ ID NO: 316 - DNA, 20 bp, synthetic construct
  misc_feature 1..20: note = Description of Artificial Sequence: Synthetic oligonucleotide
  source 1..20: mol_type = other DNA
gcactgccaagctatcagat

SEQ ID NO: 317 - DNA, 20 bp, synthetic construct
  misc_feature 1..20: note = Description of Artificial Sequence: Synthetic oligonucleotide
  source 1..20: mol_type = other DNA
tgccaagctatcagataagc

SEQ ID NO: 318 - DNA, 20 bp, synthetic construct
  misc_feature 1..20: note = Description of Artificial Sequence: Synthetic oligonucleotide
  source 1..20: mol_type = other DNA
ccagtggagtactatggact

SEQ ID NO: 319 - DNA, 20 bp, synthetic construct
  misc_feature 1..20: note = Description of Artificial Sequence: Synthetic oligonucleotide
  source 1..20: mol_type = other DNA
ctaatcaatgtgctaagccc

SEQ ID NO: 320 - DNA, 20 bp, synthetic construct
  misc_feature 1..20: note = Description of Artificial Sequence: Synthetic oligonucleotide
  source 1..20: mol_type = other DNA
gcacagaattagcccatact

SEQ ID NO: 321 - DNA, 20 bp, synthetic construct
  misc_feature 1..20: note = Description of Artificial Sequence: Synthetic oligonucleotide
  source 1..20: mol_type = other DNA
gaattagcccatactcaagt

SEQ ID NO: 322 - DNA, 20 bp, synthetic construct
  misc_feature 1..20: note = Description of Artificial Sequence: Synthetic oligonucleotide
  source 1..20: mol_type = other DNA
cttagaagtccccaagtcta

SEQ ID NO: 323 - DNA, 20 bp, synthetic construct
  misc_feature 1..20: note = Description of Artificial Sequence: Synthetic oligonucleotide
  source 1..20: mol_type = other DNA
cctcaataaccatgacaggt

SEQ ID NO: 324 - DNA, 20 bp, synthetic construct
  misc_feature 1..20: note = Description of Artificial Sequence: Synthetic oligonucleotide
  source 1..20: mol_type = other DNA
tcaggtactcttcagatacc

SEQ ID NO: 325 - DNA, 20 bp, synthetic construct
  misc_feature 1..20: note = Description of Artificial Sequence: Synthetic oligonucleotide
  source 1..20: mol_type = other DNA
gctcttacattcaccagata

SEQ ID NO: 326 - DNA, 20 bp, synthetic construct
  misc_feature 1..20: note = Description of Artificial Sequence: Synthetic oligonucleotide
  source 1..20: mol_type = other DNA
ttgcttaaccaatgctggtg

SEQ ID NO: 327 - DNA, 20 bp, synthetic construct
  misc_feature 1..20: note = Description of Artificial Sequence: Synthetic oligonucleotide
  source 1..20: mol_type = other DNA
gcatacagtttgcctaaacc

SEQ ID NO: 328 - DNA, 20 bp, synthetic construct
  misc_feature 1..20: note = Description of Artificial Sequence: Synthetic oligonucleotide
  source 1..20: mol_type = other DNA
cacaatcccatgataggact

SEQ ID NO: 329 - DNA, 20 bp, synthetic construct
  misc_feature 1..20: note = Description of Artificial Sequence: Synthetic oligonucleotide
  source 1..20: mol_type = other DNA
caatcccatgataggactat

SEQ ID NO: 330 - DNA, 20 bp, synthetic construct
  misc_feature 1..20: note = Description of Artificial Sequence: Synthetic oligonucleotide
  source 1..20: mol_type = other DNA
tgtcaacctaacaagttggt

SEQ ID NO: 331 - DNA, 20 bp, synthetic construct
  misc_feature 1..20: note = Description of Artificial Sequence: Synthetic oligonucleotide
  source 1..20: mol_type = other DNA
gtcaacctaacaagttggtt

SEQ ID NO: 332 - DNA, 20 bp, synthetic construct
  misc_feature 1..20: note = Description of Artificial Sequence: Synthetic oligonucleotide
  source 1..20: mol_type = other DNA
tactggtaaggcagtcccaa

SEQ ID NO: 333 - DNA, 20 bp, synthetic construct
  misc_feature 1..20: note = Description of Artificial Sequence: Synthetic oligonucleotide
  source 1..20: mol_type = other DNA
caccaggtagtcccaaataa

SEQ ID NO: 334 - DNA, 20 bp, synthetic construct
  misc_feature 1..20: note = Description of Artificial Sequence: Synthetic oligonucleotide
  source 1..20: mol_type = other DNA
ggtagtcccaaataactttc

SEQ ID NO: 335 - DNA, 20 bp, synthetic construct
  misc_feature 1..20: note = Description of Artificial Sequence: Synthetic oligonucleotide
  source 1..20: mol_type = other DNA
attagcacaggtatccctac

SEQ ID NO: 336 - DNA, 20 bp, synthetic construct
  misc_feature 1..20: note = Description of Artificial Sequence: Synthetic oligonucleotide
  source 1..20: mol_type = other DNA
gtagccctcccctaaattct

SEQ ID NO: 337 - DNA, 20 bp, synthetic construct
  misc_feature 1..20: note = Description of Artificial Sequence: Synthetic oligonucleotide
  source 1..20: mol_type = other DNA
ggtaattccacccctccaac

SEQ ID NO: 338 - DNA, 20 bp, synthetic construct
  misc_feature 1..20: note = Description of Artificial Sequence: Synthetic oligonucleotide
  source 1..20: mol_type = other DNA
gtcaaagacctgagtcacct

SEQ ID NO: 339 - DNA, 20 bp, synthetic construct
  misc_feature 1..20: note = Description of Artificial Sequence: Synthetic oligonucleotide
  source 1..20: mol_type = other DNA
tgtgagagatcaactcaaca

SEQ ID NO: 340 - DNA, 20 bp, synthetic construct
  misc_feature 1..20: note = Description of Artificial Sequence: Synthetic oligonucleotide
  source 1..20: mol_type = other DNA
ggagtattactgccccaaaa

SEQ ID NO: 341 - DNA, 20 bp, synthetic construct
  misc_feature 1..20: note = Description of Artificial Sequence: Synthetic oligonucleotide
  source 1..20: mol_type = other DNA
ctagagaccacttaacctcc

SEQ ID NO: 342 - DNA, 20 bp, synthetic construct
  misc_feature 1..20: note = Description of Artificial Sequence: Synthetic oligonucleotide
  source 1..20: mol_type = other DNA
gagaccacttaacctccccc

SEQ ID NO: 343 - DNA, 20 bp, synthetic construct
  misc_feature 1..20: note = Description of Artificial Sequence: Synthetic oligonucleotide
  source 1..20: mol_type = other DNA
ccccctacatcctatcacta

SEQ ID NO: 344 - DNA, 20 bp, synthetic construct
  misc_feature 1..20: note = Description of Artificial Sequence: Synthetic oligonucleotide
  source 1..20: mol_type = other DNA
ctatagtgtatccagccaat

SEQ ID NO: 345 - DNA, 20 bp, synthetic construct
  misc_feature 1..20: note = Description of Artificial Sequence: Synthetic oligonucleotide
  source 1..20: mol_type = other DNA
ggatggtgtaggttccaaac

SEQ ID NO: 346 - DNA, 20 bp, synthetic construct
  misc_feature 1..20: note = Description of Artificial Sequence: Synthetic oligonucleotide
  source 1..20: mol_type = other DNA
gatggtgtaggttccaaacc

SEQ ID NO: 347 - DNA, 20 bp, synthetic construct
  misc_feature 1..20: note = Description of Artificial Sequence: Synthetic oligonucleotide
  source 1..20: mol_type = other DNA
tgttaccacatttcagtccc

SEQ ID NO: 348 - DNA, 20 bp, synthetic construct
  misc_feature 1..20: note = Description of Artificial Sequence: Synthetic oligonucleotide
  source 1..20: mol_type = other DNA
aacttccaagagttagcacc

SEQ ID NO: 349 - DNA, 20 bp, synthetic construct
  misc_feature 1..20: note = Description of Artificial Sequence: Synthetic oligonucleotide
  source 1..20: mol_type = other DNA
agtaccctccaaagaatttc

SEQ ID NO: 350 - DNA, 20 bp, synthetic construct
  misc_feature 1..20: note = Description of Artificial Sequence: Synthetic oligonucleotide
  source 1..20: mol_type = other DNA
gcctaaccagtcagctatta

SEQ ID NO: 351 - DNA, 20 bp, synthetic construct
  misc_feature 1..20: note = Description of Artificial Sequence: Synthetic oligonucleotide
  source 1..20: mol_type = other DNA
ccagtcagctattagaacta

SEQ ID NO: 352 - DNA, 20 bp, synthetic construct
  misc_feature 1..20: note = Description of Artificial Sequence: Synthetic oligonucleotide
  source 1..20: mol_type = other DNA
ggttttgtcaagctaacaac

SEQ ID NO: 353 - DNA, 20 bp, synthetic construct
  misc_feature 1..20: note = Description of Artificial Sequence: Synthetic oligonucleotide
  source 1..20: mol_type = other DNA
ggctgtaatgacctcccctg

SEQ ID NO: 354 - DNA, 20 bp, synthetic construct
  misc_feature 1..20: note = Description of Artificial Sequence: Synthetic oligonucleotide
  source 1..20: mol_type = other DNA
ctcaggatctactcctacta

SEQ ID NO: 355 - DNA, 20 bp, synthetic construct
  misc_feature 1..20: note = Description of Artificial Sequence: Synthetic oligonucleotide
  source 1..20: mol_type = other DNA
aggatctactcctactactc

SEQ ID NO: 356 - DNA, 20 bp, synthetic construct
  misc_feature 1..20: note = Description of Artificial Sequence: Synthetic oligonucleotide
  source 1..20: mol_type = other DNA
gaagcctgacccctatactt

SEQ ID NO: 357 - DNA, 20 bp, synthetic construct
  misc_feature 1..20: note = Description of Artificial Sequence: Synthetic oligonucleotide
  source 1..20: mol_type = other DNA
gtaaaacctgacctatgtcc

SEQ ID NO: 358 - DNA, 20 bp, synthetic construct
  misc_feature 1..20: note = Description of Artificial Sequence: Synthetic oligonucleotide
  source 1..20: mol_type = other DNA
gcttaaacccatcaaactga

SEQ ID NO: 359 - DNA, 20 bp, synthetic construct
  misc_feature 1..20: note = Description of Artificial Sequence: Synthetic oligonucleotide
  source 1..20: mol_type = other DNA
tccaaatggctgatgtaacc

SEQ ID NO: 360 - DNA, 20 bp, synthetic construct
  misc_feature 1..20: note = Description of Artificial Sequence: Synthetic oligonucleotide
  source 1..20: mol_type = other DNA
cagcccaataatggagttga

SEQ ID NO: 361 - DNA, 20 bp, synthetic construct
  misc_feature 1..20: note = Description of Artificial Sequence: Synthetic oligonucleotide
  source 1..20: mol_type = other DNA
ggtctgtttactggacagat

SEQ ID NO: 362 - DNA, 20 bp, synthetic construct
  misc_feature 1..20: note = Description of Artificial Sequence: Synthetic oligonucleotide
  source 1..20: mol_type = other DNA
cagatcccttacactatcaa

SEQ ID NO: 363 - DNA, 20 bp, synthetic construct
  misc_feature 1..20: note = Description of Artificial Sequence: Synthetic oligonucleotide
  source 1..20: mol_type = other DNA
atccagacctttagttacca

SEQ ID NO: 364 - DNA, 20 bp, synthetic construct
  misc_feature 1..20: note = Description of Artificial Sequence: Synthetic oligonucleotide
  source 1..20: mol_type = other DNA
aatcataagagccccaatca

SEQ ID NO: 365 - DNA, 20 bp, synthetic construct
  misc_feature 1..20: note = Description of Artificial Sequence: Synthetic oligonucleotide
  source 1..20: mol_type = other DNA
gatctgatagccaactcaat

SEQ ID NO: 366 - DNA, 20 bp, synthetic construct
  misc_feature 1..20: note = Description of Artificial Sequence: Synthetic oligonucleotide
  source 1..20: mol_type = other DNA
ggatggcaagatcccataca

SEQ ID NO: 367 - DNA, 20 bp, synthetic construct
  misc_feature 1..20: note = Description of Artificial Sequence: Synthetic oligonucleotide
  source 1..20: mol_type = other DNA
gaaccccaagagtgatgacc

SEQ ID NO: 368 - DNA, 20 bp, synthetic construct
  misc_feature 1..20: note = Description of Artificial Sequence: Synthetic oligonucleotide
  source 1..20: mol_type = other DNA
cctgagtggtaatcagtcct

SEQ ID NO: 369 - DNA, 20 bp, synthetic construct
  misc_feature 1..20: note = Description of Artificial Sequence: Synthetic oligonucleotide
  source 1..20: mol_type = other DNA
taacctacctacccaaagcc

SEQ ID NO: 370 - DNA, 20 bp, synthetic construct
  misc_feature 1..20: note = Description of Artificial Sequence: Synthetic oligonucleotide
  source 1..20: mol_type = other DNA
gcactagatggtttgaatac

SEQ ID NO: 371 - DNA, 20 bp, synthetic construct
  misc_feature 1..20: note = Description of Artificial Sequence: Synthetic oligonucleotide
  source 1..20: mol_type = other DNA
gatcaggctcattcacaaca

SEQ ID NO: 372 - DNA, 20 bp, synthetic construct
  misc_feature 1..20: note = Description of Artificial Sequence: Synthetic oligonucleotide
  source 1..20: mol_type = other DNA
gccacaaagttacctactag

SEQ ID NO: 373 - DNA, 20 bp, synthetic construct
  misc_feature 1..20: note = Description of Artificial Sequence: Synthetic oligonucleotide
  source 1..20: mol_type = other DNA
ggaacttagcatcacattag

SEQ ID NO: 374 - DNA, 20 bp, synthetic construct
  misc_feature 1..20: note = Description of Artificial Sequence: Synthetic oligonucleotide
  source 1..20: mol_type = other DNA
gacagctccacaggtatcaa

SEQ ID NO: 375 - DNA, 20 bp, synthetic construct
  misc_feature 1..20: note = Description of Artificial Sequence: Synthetic oligonucleotide
  source 1..20: mol_type = other DNA
gctattattagaccaggttc

SEQ ID NO: 376 - DNA, 20 bp, synthetic construct
  misc_feature 1..20: note = Description of Artificial Sequence: Synthetic oligonucleotide
  source 1..20: mol_type = other DNA
gacttgagtaccaggaccag

SEQ ID NO: 377 - DNA, 20 bp, synthetic construct
  misc_feature 1..20: note = Description of Artificial Sequence: Synthetic oligonucleotide
  source 1..20: mol_type = other DNA
ttgagtaccaggaccagtac

SEQ ID NO: 378 - DNA, 20 bp, synthetic construct
  misc_feature 1..20: note = Description of Artificial Sequence: Synthetic oligonucleotide
  source 1..20: mol_type = other DNA
cagttcaaggactagctctt

SEQ ID NO: 379 - DNA, 20 bp, synthetic construct
  misc_feature 1..20: note = Description of Artificial Sequence: Synthetic oligonucleotide
  source 1..20: mol_type = other DNA
cacatagtccaataccctta

SEQ ID NO: 380 - DNA, 20 bp, synthetic construct
  misc_feature 1..20: note = Description of Artificial Sequence: Synthetic oligonucleotide
  source 1..20: mol_type = other DNA
atagtccaatacccttacct

SEQ ID NO: 381 - DNA, 20 bp, synthetic construct
  misc_feature 1..20: note = Description of Artificial Sequence: Synthetic oligonucleotide
  source 1..20: mol_type = other DNA
ctgccattaggactgttaca

SEQ ID NO: 382 - DNA, 20 bp, synthetic construct
  misc_feature 1..20: note = Description of Artificial Sequence: Synthetic oligonucleotide
  source 1..20: mol_type = other DNA
tgccattaggactgttacag

SEQ ID NO: 383 - DNA, 20 bp, synthetic construct
  misc_feature 1..20: note = Description of Artificial Sequence: Synthetic oligonucleotide
  source 1..20: mol_type = other DNA
aaggctcacctataaacaac

SEQ ID NO: 384 - DNA, 20 bp, synthetic construct
  misc_feature 1..20: note = Description of Artificial Sequence: Synthetic oligonucleotide
  source 1..20: mol_type = other DNA
gtatgaccatccttacatac

SEQ ID NO: 385 - DNA, 20 bp, synthetic construct
  misc_feature 1..20: note = Description of Artificial Sequence: Synthetic oligonucleotide
  source 1..20: mol_type = other DNA
aagtagttggactctcccag

SEQ ID NO: 386 - DNA, 20 bp, synthetic construct
  misc_feature 1..20: note = Description of Artificial Sequence: Synthetic oligonucleotide
  source 1..20: mol_type = other DNA
agttggactctcccagtgcc

SEQ ID NO: 387 - DNA, 20 bp, synthetic construct
  misc_feature 1..20: note = Description of Artificial Sequence: Synthetic oligonucleotide
  source 1..20: mol_type = other DNA
gtacaggtcactacagatga

SEQ ID NO: 388 - DNA, 20 bp, synthetic construct
  misc_feature 1..20: note = Description of Artificial Sequence: Synthetic oligonucleotide
  source 1..20: mol_type = other DNA
taatatccactcctacctgg

SEQ ID NO: 389 - DNA, 20 bp, synthetic construct
  misc_feature 1..20: note = Description of Artificial Sequence: Synthetic oligonucleotide
  source 1..20: mol_type = other DNA
cctacctaagcccacctaca

SEQ ID NO: 390 - DNA, 20 bp, synthetic construct
  misc_feature 1..20: note = Description of Artificial Sequence: Synthetic oligonucleotide
  source 1..20: mol_type = other DNA
gtgtagctccatttcaactt

SEQ ID NO: 391 - DNA, 20 bp, synthetic construct
  misc_feature 1..20: note = Description of Artificial Sequence: Synthetic oligonucleotide
  source 1..20: mol_type = other DNA
ggtgctacttactgaacacc

SEQ ID NO: 392 - DNA, 20 bp, synthetic construct
  misc_feature 1..20: note = Description of Artificial Sequence: Synthetic oligonucleotide
  source 1..20: mol_type = other DNA
ccctaactgtatcttcccaa

SEQ ID NO: 393 - DNA, 20 bp, synthetic construct
  misc_feature 1..20: note = Description of Artificial Sequence: Synthetic oligonucleotide
  source 1..20: mol_type = other DNA
cactggctagtgctttatcc

SEQ ID NO: 394 - DNA, 20 bp, synthetic construct
  misc_feature 1..20: note = Description of Artificial Sequence: Synthetic oligonucleotide
  source 1..20: mol_type = other DNA
gcaactgtcaaccaagactg

SEQ ID NO: 395 - DNA, 20 bp, synthetic construct
  misc_feature 1..20: note = Description of Artificial Sequence: Synthetic oligonucleotide
  source 1..20: mol_type = other DNA
gtaatccaggtcatagttcc

SEQ ID NO: 396 - DNA, 20 bp, synthetic construct
  misc_feature 1..20: note = Description of Artificial Sequence: Synthetic oligonucleotide
  source 1..20: mol_type = other DNA
ccaatagtgacttatggcat

SEQ ID NO: 397 - DNA, 20 bp, synthetic construct
  misc_feature 1..20: note = Description of Artificial Sequence: Synthetic oligonucleotide
  source 1..20: mol_type = other DNA
ccaggcagcaacctacctga

SEQ ID NO: 398 - DNA, 20 bp, synthetic construct
  misc_feature 1..20: note = Description of Artificial Sequence: Synthetic oligonucleotide
  source 1..20: mol_type = other DNA
ggagactgctaaggctacta

SEQ ID NO: 399 - DNA, 20 bp, synthetic construct
  misc_feature 1..20: note = Description of Artificial Sequence: Synthetic oligonucleotide
  source 1..20: mol_type = other DNA
agactgctaaggctactaca

SEQ ID NO: 400 - DNA, 20 bp, synthetic construct
  misc_feature 1..20: note = Description of Artificial Sequence: Synthetic oligonucleotide
  source 1..20: mol_type = other DNA
gcattaccaataaatccacc

SEQ ID NO: 401 - DNA, 20 bp, synthetic construct
  misc_feature 1..20: note = Description of Artificial Sequence: Synthetic oligonucleotide
  source 1..20: mol_type = other DNA
tggtcagtctacccagttaa

SEQ ID NO: 402 - DNA, 20 bp, synthetic construct
  misc_feature 1..20: note = Description of Artificial Sequence: Synthetic oligonucleotide
  source 1..20: mol_type = other DNA
gcccttctcacattaagcat

SEQ ID NO: 403 - DNA, 20 bp, synthetic construct
  misc_feature 1..20: note = Description of Artificial Sequence: Synthetic oligonucleotide
  source 1..20: mol_type = other DNA
aattaccatgccccagagac

SEQ ID NO: 404 - DNA, 20 bp, synthetic construct
  misc_feature 1..20: note = Description of Artificial Sequence: Synthetic oligonucleotide
  source 1..20: mol_type = other DNA
cctaaatacaggtcctaatg

SEQ ID NO: 405 - DNA, 20 bp, synthetic construct
  misc_feature 1..20: note = Description of Artificial Sequence: Synthetic oligonucleotide
  source 1..20: mol_type = other DNA
aacatcaatctgagacaccc

SEQ ID NO: 406 - DNA, 20 bp, synthetic construct
  misc_feature 1..20: note = Description of Artificial Sequence: Synthetic oligonucleotide
  source 1..20: mol_type = other DNA
gttgcctatcctcacttacc

SEQ ID NO: 407 - DNA, 20 bp, synthetic construct
  misc_feature 1..20: note = Description of Artificial Sequence: Synthetic oligonucleotide
  source 1..20: mol_type = other DNA
cctcaggtcaatctatgcta

SEQ ID NO: 408 - DNA, 20 bp, synthetic construct
  misc_feature 1..20: note = Description of Artificial Sequence: Synthetic oligonucleotide
  source 1..20: mol_type = other DNA
tatgcccactgaaaccttac

SEQ ID NO: 409 - DNA, 20 bp, synthetic construct
  misc_feature 1..20: note = Description of Artificial Sequence: Synthetic oligonucleotide
  source 1..20: mol_type = other DNA
aggttacttcacaaactcca

SEQ ID NO: 410 - DNA, 20 bp, synthetic construct
  misc_feature 1..20: note = Description of Artificial Sequence: Synthetic oligonucleotide
  source 1..20: mol_type = other DNA
gcccatattacaccttagga

SEQ ID NO: 411 - DNA, 20 bp, synthetic construct
  misc_feature 1..20: note = Description of Artificial Sequence: Synthetic oligonucleotide
  source 1..20: mol_type = other DNA
ataactaaggtgccccaact

SEQ ID NO: 412 - DNA, 20 bp, synthetic construct
  misc_feature 1..20: note = Description of Artificial Sequence: Synthetic oligonucleotide
  source 1..20: mol_type = other DNA
atcttccactaagaagtccc

SEQ ID NO: 413 - DNA, 20 bp, synthetic construct
  misc_feature 1..20: note = Description of Artificial Sequence: Synthetic oligonucleotide
  source 1..20: mol_type = other DNA
tgctaagccctaagtatatc

SEQ ID NO: 414 - DNA, 20 bp, synthetic construct
  misc_feature 1..20: note = Description of Artificial Sequence: Synthetic oligonucleotide
  source 1..20: mol_type = other DNA
aagccctaagtatatcctca

SEQ ID NO: 415 - DNA, 20 bp, synthetic construct
  misc_feature 1..20: note = Description of Artificial Sequence: Synthetic oligonucleotide
  source 1..20: mol_type = other DNA
gtatctatatgagactccag

SEQ ID NO: 416 - DNA, 20 bp, synthetic construct
  misc_feature 1..20: note = Description of Artificial Sequence: Synthetic oligonucleotide
  source 1..20: mol_type = other DNA
tataccatccacctgagttc

SEQ ID NO: 417 - DNA, 20 bp, synthetic construct
  misc_feature 1..20: note = Description of Artificial Sequence: Synthetic oligonucleotide
  source 1..20: mol_type = other DNA
gcaaaagttcactactccca

SEQ ID NO: 418 - DNA, 20 bp, synthetic construct
  misc_feature 1..20: note = Description of Artificial Sequence: Synthetic oligonucleotide
  source 1..20: mol_type = other DNA
gttcactactcccaactact

SEQ ID NO: 419 - DNA, 20 bp, synthetic construct
  misc_feature 1..20: note = Description of Artificial Sequence: Synthetic oligonucleotide
  source 1..20: mol_type = other DNA
aggtactcttcagataccta

SEQ ID NO: 420 - DNA, 20 bp, synthetic construct
  misc_feature 1..20: note = Description of Artificial Sequence: Synthetic oligonucleotide
  source 1..20: mol_type = other DNA
gtccctataacataacctag

SEQ ID NO: 421 - DNA, 20 bp, synthetic construct
  misc_feature 1..20: note = Description of Artificial Sequence: Synthetic oligonucleotide
  source 1..20: mol_type = other DNA
caagtgaccaccatctatag

SEQ ID NO: 422 - DNA, 20 bp, synthetic construct
  misc_feature 1..20: note = Description of Artificial Sequence: Synthetic oligonucleotide
  source 1..20: mol_type = other DNA
gctagtcaagtctcattaac

SEQ ID NO: 423 - DNA, 20 bp, synthetic construct
  misc_feature 1..20: note = Description of Artificial Sequence: Synthetic oligonucleotide
  source 1..20: mol_type = other DNA
ttacacaatcccatgatagg

SEQ ID NO: 424 - DNA, 20 bp, synthetic construct
  misc_feature 1..20: note = Description of Artificial Sequence: Synthetic oligonucleotide
  source 1..20: mol_type = other DNA
acaatcccatgataggacta

SEQ ID NO: 425 - DNA, 20 bp, synthetic construct
  misc_feature 1..20: note = Description of Artificial Sequence: Synthetic oligonucleotide
  source 1..20: mol_type = other DNA
cccaatctagttcaagcatc

SEQ ID NO: 426 - DNA, 20 bp, synthetic construct
  misc_feature 1..20: note = Description of Artificial Sequence: Synthetic oligonucleotide
  source 1..20: mol_type = other DNA
agatggtgactacctcctac

SEQ ID NO: 427 - DNA, 20 bp, synthetic construct
  misc_feature 1..20: note = Description of Artificial Sequence: Synthetic oligonucleotide
  source 1..20: mol_type = other DNA
ctggtaaggcagtcccaaca

SEQ ID NO: 428 - DNA, 20 bp, synthetic construct
  misc_feature 1..20: note = Description of Artificial Sequence: Synthetic oligonucleotide
  source 1..20: mol_type = other DNA
taaggcagtcccaacaaaac

SEQ ID NO: 429 - DNA, 20 bp, synthetic construct
  misc_feature 1..20: note = Description of Artificial Sequence: Synthetic oligonucleotide
  source 1..20: mol_type = other DNA
aagcaggacctgtctgttac

SEQ ID NO: 430 - DNA, 20 bp, synthetic construct
  misc_feature 1..20: note = Description of Artificial Sequence: Synthetic oligonucleotide
  source 1..20: mol_type = other DNA
gacaggcatagcttatggat

SEQ ID NO: 431 - DNA, 20 bp, synthetic construct
  misc_feature 1..20: note = Description of Artificial Sequence: Synthetic oligonucleotide
  source 1..20: mol_type = other DNA
ctagattagtgaacagtagc

SEQ ID NO: 432 - DNA, 20 bp, synthetic construct
  misc_feature 1..20: note = Description of Artificial Sequence: Synthetic oligonucleotide
  source 1..20: mol_type = other DNA
cacactaacctaaggtaact

SEQ ID NO: 433 - DNA, 20 bp, synthetic construct
  misc_feature 1..20: note = Description of Artificial Sequence: Synthetic oligonucleotide
  source 1..20: mol_type = other DNA
ccaggtagtcccaaataact

SEQ ID NO: 434 - DNA, 20 bp, synthetic construct
  misc_feature 1..20: note = Description of Artificial Sequence: Synthetic oligonucleotide
  source 1..20: mol_type = other DNA
gtagtcccaaataactttcc

SEQ ID NO: 435 - DNA, 20 bp, synthetic construct
  misc_feature 1..20: note = Description of Artificial Sequence: Synthetic oligonucleotide
  source 1..20: mol_type = other DNA
attgcccccagactcctact

SEQ ID NO: 436 - DNA, 20 bp, synthetic construct
  misc_feature 1..20: note = Description of Artificial Sequence: Synthetic oligonucleotide
  source 1..20: mol_type = other DNA
ccccagactcctacttaaac

SEQ ID NO: 437 - DNA, 20 bp, synthetic construct
  misc_feature 1..20: note = Description of Artificial Sequence: Synthetic oligonucleotide
  source 1..20: mol_type = other DNA
gagtatagaatacttgaccc

SEQ ID NO: 438 - DNA, 20 bp, synthetic construct
  misc_feature 1..20: note = Description of Artificial Sequence: Synthetic oligonucleotide
  source 1..20: mol_type = other DNA
gtaaattagcacaggtatcc

SEQ ID NO: 439 - DNA, 20 bp, synthetic construct
  misc_feature 1..20: note = Description of Artificial Sequence: Synthetic oligonucleotide
  source 1..20: mol_type = other DNA
actgttcctcccagttggta

SEQ ID NO: 440 - DNA, 20 bp, synthetic construct
  misc_feature 1..20: note = Description of Artificial Sequence: Synthetic oligonucleotide
  source 1..20: mol_type = other DNA
gttcctcccagttggtaatt

SEQ ID NO: 441 - DNA, 20 bp, synthetic construct
  misc_feature 1..20: note = Description of Artificial Sequence: Synthetic oligonucleotide
  source 1..20: mol_type = other DNA
ctcccagttggtaattccac

SEQ ID NO: 442 - DNA, 20 bp, synthetic construct
  misc_feature 1..20: note = Description of Artificial Sequence: Synthetic oligonucleotide
  source 1..20: mol_type = other DNA
cagttggtaattccacccct

SEQ ID NO: 443 - DNA, 20 bp, synthetic construct
  misc_feature 1..20: note = Description of Artificial Sequence: Synthetic oligonucleotide
  source 1..20: mol_type = other DNA
agttggtaattccacccctc

SEQ ID NO: 444 - DNA, 20 bp, synthetic construct
  misc_feature 1..20: note = Description of Artificial Sequence: Synthetic oligonucleotide
  source 1..20: mol_type = other DNA
gtaccctgatgatacccttc

SEQ ID NO: 445 - DNA, 20 bp, synthetic construct
  misc_feature 1..20: note = Description of Artificial Sequence: Synthetic oligonucleotide
  source 1..20: mol_type = other DNA
ggttagaatggatcaaaacc

SEQ ID NO: 446 - DNA, 20 bp, synthetic construct
  misc_feature 1..20: note = Description of Artificial Sequence: Synthetic oligonucleotide
  source 1..20: mol_type = other DNA
gtcaaagtaggtccactgaa

SEQ ID NO: 447 - DNA, 20 bp, synthetic construct
  misc_feature 1..20: note = Description of Artificial Sequence: Synthetic oligonucleotide
  source 1..20: mol_type = other DNA
acttccaagagttagcaccc

SEQ ID NO: 448 - DNA, 20 bp, synthetic construct
  misc_feature 1..20: note = Description of Artificial Sequence: Synthetic oligonucleotide
  source 1..20: mol_type = other DNA
accctctactaccaagcagt

SEQ ID NO: 449 - DNA, 20 bp, synthetic construct
  misc_feature 1..20: note = Description of Artificial Sequence: Synthetic oligonucleotide
  source 1..20: mol_type = other DNA
ggtgtccaaactaccataaa

SEQ ID NO: 450 - DNA, 20 bp, synthetic construct
  misc_feature 1..20: note = Description of Artificial Sequence: Synthetic oligonucleotide
  source 1..20: mol_type = other DNA
ctaaccagtcagctattaga

SEQ ID NO: 451 - DNA, 20 bp, synthetic construct
  misc_feature 1..20: note = Description of Artificial Sequence: Synthetic oligonucleotide
  source 1..20: mol_type = other DNA
atcagcaagagcctattaag

SEQ ID NO: 452 - DNA, 20 bp, synthetic construct
  misc_feature 1..20: note = Description of Artificial Sequence: Synthetic oligonucleotide
  source 1..20: mol_type = other DNA
gcaggttcagtgctaagatt

SEQ ID NO: 453 - DNA, 20 bp, synthetic construct
  misc_feature 1..20: note = Description of Artificial Sequence: Synthetic oligonucleotide
  source 1..20: mol_type = other DNA
gtaagacttgctgactcacc

SEQ ID NO: 454 - DNA, 20 bp, synthetic construct
  misc_feature 1..20: note = Description of Artificial Sequence: Synthetic oligonucleotide
  source 1..20: mol_type = other DNA
caagtagctgtaagtccaac

SEQ ID NO: 455 - DNA, 20 bp, synthetic construct
  misc_feature 1..20: note = Description of Artificial Sequence: Synthetic oligonucleotide
  source 1..20: mol_type = other DNA
atccccaaagagacctatgc

SEQ ID NO: 456 - DNA, 20 bp, synthetic construct
  misc_feature 1..20: note = Description of Artificial Sequence: Synthetic oligonucleotide
  source 1..20: mol_type = other DNA
caaccagcagaatccctata

SEQ ID NO: 457 - DNA, 20 bp, synthetic construct
  misc_feature 1..20: note = Description of Artificial Sequence: Synthetic oligonucleotide
  source 1..20: mol_type = other DNA
ttaggtatggcatgatcccc

SEQ ID NO: 458 - DNA, 20 bp, synthetic construct
  misc_feature 1..20: note = Description of Artificial Sequence: Synthetic oligonucleotide
  source 1..20: mol_type = other DNA
taagccctttggtccaacct

SEQ ID NO: 459 - DNA, 20 bp, synthetic construct
  misc_feature 1..20: note = Description of Artificial Sequence: Synthetic oligonucleotide
  source 1..20: mol_type = other DNA
aagccctttggtccaaccta

SEQ ID NO: 460 - DNA, 20 bp, synthetic construct
  misc_feature 1..20: note = Description of Artificial Sequence: Synthetic oligonucleotide
  source 1..20: mol_type = other DNA
taccaatggcaaggtttggc

SEQ ID NO: 461 - DNA, 20 bp, synthetic construct
  misc_feature 1..20: note = Description of Artificial Sequence: Synthetic oligonucleotide
  source 1..20: mol_type = other DNA
ttgttctggctgaccaagtc

SEQ ID NO: 462 - DNA, 20 bp, synthetic construct
  misc_feature 1..20: note = Description of Artificial Sequence: Synthetic oligonucleotide
  source 1..20: mol_type = other DNA
gttctggctgaccaagtctg

SEQ ID NO: 463 - DNA, 20 bp, synthetic construct
  misc_feature 1..20: note = Description of Artificial Sequence: Synthetic oligonucleotide
  source 1..20: mol_type = other DNA
tcaccttgacttaggcaaac

SEQ ID NO: 464 - DNA, 20 bp, synthetic construct
  misc_feature 1..20: note = Description of Artificial Sequence: Synthetic oligonucleotide
  source 1..20: mol_type = other DNA
ggcaaaccactccaagttga

SEQ ID NO: 465 - DNA, 20 bp, synthetic construct
  misc_feature 1..20: note = Description of Artificial Sequence: Synthetic oligonucleotide
  source 1..20: mol_type = other DNA
caagtggcagcccaataatg

SEQ ID NO: 466 - DNA, 20 bp, synthetic construct
  misc_feature 1..20: note = Description of Artificial Sequence: Synthetic oligonucleotide
  source 1..20: mol_type = other DNA
tttactggacagatccctta

SEQ ID NO: 467 - DNA, 20 bp, synthetic construct
  misc_feature 1..20: note = Description of Artificial Sequence: Synthetic oligonucleotide
  source 1..20: mol_type = other DNA
tactggacagatcccttaca

SEQ ID NO: 468 - DNA, 20 bp, synthetic construct
  misc_feature 1..20: note = Description of Artificial Sequence: Synthetic oligonucleotide
  source 1..20: mol_type = other DNA
ggacagatcccttacactat

SEQ ID NO: 469 - DNA, 20 bp, synthetic construct
  misc_feature 1..20: note = Description of Artificial Sequence: Synthetic oligonucleotide
  source 1..20: mol_type = other DNA
ctgaacagagtactaaccac

SEQ ID NO: 470 - DNA, 20 bp, synthetic construct
  misc_feature 1..20: note = Description of Artificial Sequence: Synthetic oligonucleotide
  source 1..20: mol_type = other DNA
ataagagccccaatcatcta

SEQ ID NO: 471 - DNA, 20 bp, synthetic construct
  misc_feature 1..20: note = Description of Artificial Sequence: Synthetic oligonucleotide
  source 1..20: mol_type = other DNA
cctaaacacatgaacctggt

SEQ ID NO: 472 - DNA, 20 bp, synthetic construct
  misc_feature 1..20: note = Description of Artificial Sequence: Synthetic oligonucleotide
  source 1..20: mol_type = other DNA
taatgtagactggtccaggc

SEQ ID NO: 473 - DNA, 20 bp, synthetic construct
  misc_feature 1..20: note = Description of Artificial Sequence: Synthetic oligonucleotide
  source 1..20: mol_type = other DNA
aaggagcccccagttgatct

SEQ ID NO: 474 - DNA, 20 bp, synthetic construct
  misc_feature 1..20: note = Description of Artificial Sequence: Synthetic oligonucleotide
  source 1..20: mol_type = other DNA
gatcccatacagccagtttg

SEQ ID NO: 475 - DNA, 20 bp, synthetic construct
  misc_feature 1..20: note = Description of Artificial Sequence: Synthetic oligonucleotide
  source 1..20: mol_type = other DNA
ttgaggcaaccaaggtactc

SEQ ID NO: 476 - DNA, 20 bp, synthetic construct
  misc_feature 1..20: note = Description of Artificial Sequence: Synthetic oligonucleotide
  source 1..20: mol_type = other DNA
caaccaaggtactctgaacc

SEQ ID NO: 477 - DNA, 20 bp, synthetic construct
  misc_feature 1..20: note = Description of Artificial Sequence: Synthetic oligonucleotide
  source 1..20: mol_type = other DNA
ccaaggtactctgaacctaa

SEQ ID NO: 478 - DNA, 20 bp, synthetic construct
  misc_feature 1..20: note = Description of Artificial Sequence: Synthetic oligonucleotide
  source 1..20: mol_type = other DNA
agtagaactgtcactatacc

SEQ ID NO: 479 - DNA, 20 bp, synthetic construct
  misc_feature 1..20: note = Description of Artificial Sequence: Synthetic oligonucleotide
  source 1..20: mol_type = other DNA
gtaacaaccacccctggaaa

SEQ ID NO: 480 - DNA, 20 bp, synthetic construct
  misc_feature 1..20: note = Description of Artificial Sequence: Synthetic oligonucleotide
  source 1..20: mol_type = other DNA
ttcctgagtggtaatcagtc

SEQ ID NO: 481 - DNA, 20 bp, synthetic construct
  misc_feature 1..20: note = Description of Artificial Sequence: Synthetic oligonucleotide
  source 1..20: mol_type = other DNA
gactacctctattgtcaaca

SEQ ID NO: 482 - DNA, 20 bp, synthetic construct
  misc_feature 1..20: note = Description of Artificial Sequence: Synthetic oligonucleotide
  source 1..20: mol_type = other DNA
acaccttgtacttcaagtgc

SEQ ID NO: 483 - DNA, 20 bp, synthetic construct
  misc_feature 1..20: note = Description of Artificial Sequence: Synthetic oligonucleotide
  source 1..20: mol_type = other DNA
cttagtagtcagaactagcc

SEQ ID NO: 484 - DNA, 20 bp, synthetic construct
  misc_feature 1..20: note = Description of Artificial Sequence: Synthetic oligonucleotide
  source 1..20: mol_type = other DNA
ggacagtctaaacagccaca

SEQ ID NO: 485 - DNA, 20 bp, synthetic construct
  misc_feature 1..20: note = Description of Artificial Sequence: Synthetic oligonucleotide
  source 1..20: mol_type = other DNA
ggaaaggttatctgttgtgc

SEQ ID NO: 486 - DNA, 20 bp, synthetic construct
  misc_feature 1..20: note = Description of Artificial Sequence: Synthetic oligonucleotide
  source 1..20: mol_type = other DNA
aaggttatctgttgtgccac

SEQ ID NO: 487 - DNA, 20 bp, synthetic construct
  misc_feature 1..20: note = Description of Artificial Sequence: Synthetic oligonucleotide
  source 1..20: mol_type = other DNA
ggacaaagtcctacctggta

SEQ ID NO: 488 - DNA, 20 bp, synthetic construct
  misc_feature 1..20: note = Description of Artificial Sequence: Synthetic oligonucleotide
  source 1..20: mol_type = other DNA
tgagtaccaggaccagtaca

SEQ ID NO: 489 - DNA, 20 bp, synthetic construct
  misc_feature 1..20: note = Description of Artificial Sequence: Synthetic oligonucleotide
  source 1..20: mol_type = other DNA
acccttacctaatcaagaga

SEQ ID NO: 490 - DNA, 20 bp, synthetic construct
  misc_feature 1..20: note = Description of Artificial Sequence: Synthetic oligonucleotide
  source 1..20: mol_type = other DNA
taaaggaagcatccccttgt

SEQ ID NO: 491 - DNA, 20 bp, synthetic construct
  misc_feature 1..20: note = Description of Artificial Sequence: Synthetic oligonucleotide
  source 1..20: mol_type = other DNA
aactgccattaggactgtta

SEQ ID NO: 492 - DNA, 20 bp, synthetic construct
  misc_feature 1..20: note = Description of Artificial Sequence: Synthetic oligonucleotide
  source 1..20: mol_type = other DNA
tgagaaaggctcacctataa

SEQ ID NO: 493 - DNA, 20 bp, synthetic construct
  misc_feature 1..20: note = Description of Artificial Sequence: Synthetic oligonucleotide
  source 1..20: mol_type = other DNA
atctatgccttccaaggatc

SEQ ID NO: 494 - DNA, 20 bp, synthetic construct
  misc_feature 1..20: note = Description of Artificial Sequence: Synthetic oligonucleotide
  source 1..20: mol_type = other DNA
gctacagcccaatagcaagc

SEQ ID NO: 495 - DNA, 20 bp, synthetic construct
  misc_feature 1..20: note = Description of Artificial Sequence: Synthetic oligonucleotide
  source 1..20: mol_type = other DNA
ggtcatcagagtctgcctat

SEQ ID NO: 496 - DNA, 20 bp, synthetic construct
  misc_feature 1..20: note = Description of Artificial Sequence: Synthetic oligonucleotide
  source 1..20: mol_type = other DNA
cctgatctaaacccataccc

SEQ ID NO: 497 - DNA, 20 bp, synthetic construct
  misc_feature 1..20: note = Description of Artificial Sequence: Synthetic oligonucleotide
  source 1..20: mol_type = other DNA
gtagttggactctcccagtg

SEQ ID NO: 498 - DNA, 20 bp, synthetic construct
  misc_feature 1..20: note = Description of Artificial Sequence: Synthetic oligonucleotide
  source 1..20: mol_type = other DNA
acagacactagtattgcctc

SEQ ID NO: 499 - DNA, 20 bp, synthetic construct
  misc_feature 1..20: note = Description of Artificial Sequence: Synthetic oligonucleotide
  source 1..20: mol_type = other DNA
aatatccactcctacctggt

SEQ ID NO: 500 - DNA, 20 bp, synthetic construct
  misc_feature 1..20: note = Description of Artificial Sequence: Synthetic oligonucleotide
  source 1..20: mol_type = other DNA
tggttccccaccaacagaat

SEQ ID NO: 501 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
atggcatgatccccataagc

SEQ ID NO: 502 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
aggaccagtacaacctgtag

SEQ ID NO: 503 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
aggtgccccaacttgttacc

SEQ ID NO: 504 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
actaaggtgccccaacttgt

SEQ ID NO: 505 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
accaggtagtcccaaataac

SEQ ID NO: 506 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
gcctgacccctatacttgac

SEQ ID NO: 507 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
ctgacccctatacttgacag

SEQ ID NO: 508 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
ttaagccctttggtccaacc

SEQ ID NO: 509 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
gagttagcaccctctactac

SEQ ID NO: 510 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
gtcataagtccttggtcaac

SEQ ID NO: 511 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
aagcctgacccctatacttg

SEQ ID NO: 512 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
agcctgacccctatacttga

SEQ ID NO: 513 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
catcctattagcttaaaccc

SEQ ID NO: 514 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
cctgacccctatacttgaca

SEQ ID NO: 515 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
tatgatctgatagccaactc

SEQ ID NO: 516 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
gtaccaggaccagtacaacc

SEQ ID NO: 517 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
caggaccagtacaacctgta

SEQ ID NO: 518 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
acaccctttggagtgtctag

SEQ ID NO: 519 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
gccccaacttgttacctaag

SEQ ID NO: 520 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
ccccaatctagttcaagcat

SEQ ID NO: 521 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
tccaagagttagcaccctct

SEQ ID NO: 522 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
ggaccagtacaacctgtagt

SEQ ID NO: 523 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
aaacccagtgtagctccatt

SEQ ID NO: 524 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
tgccccaacttgttacctaa

SEQ ID NO: 525 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
ccaatagcaagcagcctata

SEQ ID NO: 526 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
ctacaatcaaatcctggtag

SEQ ID NO: 527 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
gaccaagtttatggacccca

SEQ ID NO: 528 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
gtgaccaccatctatagtta

SEQ ID NO: 529 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
ctaaggtaactgttcctaga

SEQ ID NO: 530 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
tgaccaagtttatggacccc

SEQ ID NO: 531 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
accaagtttatggaccccaa

SEQ ID NO: 532 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
gcaataaagctaaccacatc

SEQ ID NO: 533 - DNA, 20 bp, synthetic construct
  misc_feature 1..20: note = Description of Artificial Sequence: Synthetic oligonucleotide
  source 1..20: mol_type = other DNA
  modified_base order(1,6,7,8,9,10,11,12,13,14,15,18,19): mod_base = OTHER; note = phosphorothioate backbone substitution
  misc_difference order(1..5,16..20): note = 2'-O-methoxy-ethyl Base (2'-MOE), 2'-O-methyl (2'-OMe), or locked nucleic acid (LNA)
gattctaccacacagtacac

SEQ ID NO: 534 - DNA, 180 bp, synthetic construct
  misc_feature 1..180: note = Description of Unknown: C9ORF72 repeat expansion sequence
  source 1..180: mol_type = other DNA
ggggccggggccggggccggggccggggccggggccggggccggggccggggccggggccggggccggggccggggccggggccggggccggggccggggccggggccggggccggggccggggccggggccggggccggggccggggccggggccggggccggggccggggccggggcc